(19) United States
(12) Patent Application Publication
(10) Pub. No.: US 2024/0051933 A1
(43) Pub. Date: Feb. 15, 2024

(54) UROLITHIN DERIVATIVES AND THERAPEUTIC USES

(71) Applicant: Vandria SA, Lausanne (CH)

(72) Inventors: Peter C. Sennhenn, Munich (DE); Said Oumouch, Wittenheim (FR); Penelope Andreux, Eclepens (CH)

(21) Appl. No.: 18/226,590

(22) Filed: Jul. 26, 2023

Related U.S. Application Data
(60) Provisional application No. 63/392,611, filed on Jul. 27, 2022.

(51) Int. Cl. C07D 311/80; C07D 311/96; A61P 43/00

(52) U.S. Cl. CPC C07D 311/80; C07D 311/96; A61P 43/00

(57) ABSTRACT

Disclosed are compounds, compositions, and methods useful for inhibiting ferroptosis in a subject in need thereof.

### RELATED APPLICATION

This application claims benefit of priority to U.S. Provisional Patent Application No. 63/392,611, filed Jul. 27, 2022.

### BACKGROUND

Urolithins have potent effects on the improvement of a number of health conditions, and they have been shown to be highly biologically active in vitro and in vivo. Urolithins have been proposed as treatments of a variety of conditions including conditions related to inadequate mitochondrial activity, including obesity, memory decline, reduced metabolic rate, metabolic syndrome, diabetes mellitus, cardiovascular disease, hyperlipidemia, neurodegenerative diseases, cognitive disorder, mood disorder, stress, anxiety disorder, fatty liver disease, for improving liver function and weight management. In particular, urolithins have been shown to have beneficial effects in the enhancement of muscle function.

### SUMMARY

One aspect of the invention provides compounds, compositions, and methods useful for inhibiting ferroptosis.

Accordingly, provided herein is a compound having the structure of Formula (I):

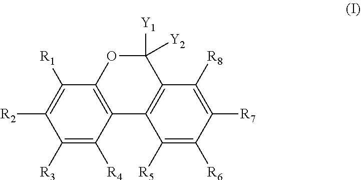

wherein
Y₁ and Y₂ are each alkyl; or taken together with the carbon to which they are bonded combine to form an unsubstituted or substituted spiro cycloalkyl;
R₁, R₄, R₅, and R₈ are independently selected from —H and halogen;
R₂ and R₇ are independently selected from —H, —OH, —OAc, —NH₂, halogen, —CN, —CF₃, —CO₂H, —NO₂, —NHAc, alkyl, haloalkyl, alkenyl, alkynyl, cycloalkyl, heterocycloalkyl, alkylamino, alkyl-R₉, alkenyl-R₉, alkynyl-R₉, —OR₁₀, —NHR₁₀, —NR₁₁C(O)R₁₂, —C(O)NR₁₁R₁₂, and —NR₁₁SO₂R₁₂;
R₃ and R₆ are independently selected from alkyl and cycloalkyl;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from alkyl, hydroxyalkyl, aminoalkyl, alkyl-O-alkyl, alkyl-O-alkyl-OH, alkyl-O-alkyl-O-alkyl, alkenyl, alkynyl, arylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of Ru is selected from H and alkyl; and
each occurrence of R₁₂ is selected from alkyl, alkenyl, alkynyl, cycloalkyl, heterocycloalkyl, O-alkyl, aminoalkyl, arylalkyl, heteroarylalkyl, alkyl-cycloalkyl, and alkyl-heterocycloalkyl;
provided that when R₁, R₄, R₅, and R₈ are each —H, R₂ and R₇ are each —OH, and R₃ and R₆ are each CH₃, then Y₁ and Y₂ are not each -Me or are not taken together with the carbon to which they are bonded to form an unsubstituted spiro cyclobutyl;
or a pharmaceutically acceptable salt thereof.

Also provided herein is a compound having the structure of Formula (II):

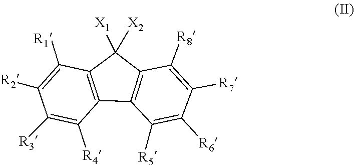

wherein
X₁ and X₂ are each alkyl; or taken together with the carbon to which they are bonded combine to form an unsubstituted or substituted spiro cycloalkyl; R₁′, R₄′, R₅′, and R₈′ are independently selected from —H, —OH, —NH₂, alkyl, and halogen;
R₂′, R₃′, R₆′, and R₇′ are independently selected from —H, —OH, —OAc, —NH₂, halogen, —CN, —CF₃, —CO₂H, —NO₂, —NHAc, alkyl, haloalkyl, alkenyl, alkynyl, cycloalkyl, heterocycloalkyl, alkylamino, and —OR₈′; and
R₈′ is selected from alkyl, hydroxyalkyl, aminoalkyl, aryl, heteroaryl, cycloalkyl, and heterocycloalkyl;
or a pharmaceutically acceptable salt thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### BRIEF DESCRIPTION OF THE DRAWINGS

FIG. 1 is a table summarizing the anti-ferroptotic activity of selected compounds. Ferroptosis EC₅₀: Half maximal effective concentration (nM) for exemplary compounds of the invention. Ferroptosis pEC₅₀: The negative logarithm of the EC₅₀ for exemplary compounds of the invention. Maximal Percent efficacy (%): Concentration at maximal Percent efficacy (M) for exemplary compounds of the invention.

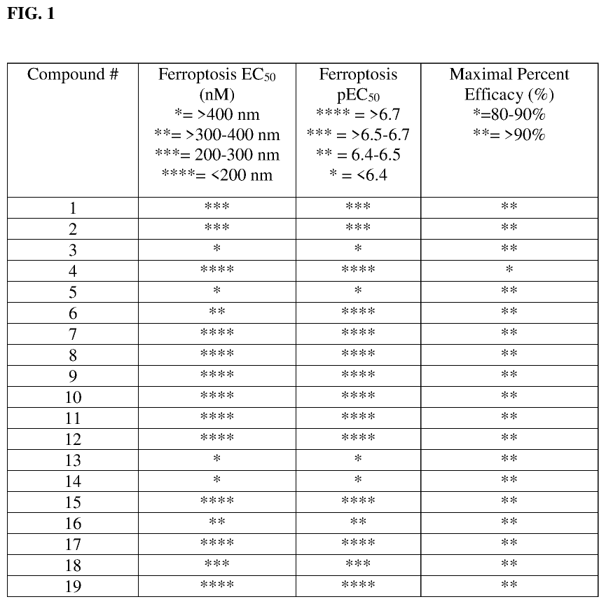

### DETAILED DESCRIPTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

Additional asymmetric carbon atoms may be present in a substituent such as an alkyl group. All such isomers, as well as mixtures thereof, are intended to be included in this invention.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ³C- or ¹⁴C-enriched carbon are within the scope of this invention.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.) In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C1-C30 for straight chains, C3-C30 for branched chains), and more preferably 20 or fewer. Alkyl groups may be substituted or unsubstituted.

As used herein, the term “heteroalkyl” refers to an alkyl moiety as hereinbefore defined which contain one or more oxygen, sulfur, nitrogen, phosphorus, or silicon atoms in place of carbon atoms.

As used herein, the term “haloalkyl” refers to an alkyl group as hereinbefore defined substituted with at least one halogen.

As used herein, the term “hydroxyalkyl” refers to an alkyl group as hereinbefore defined substituted with at least one hydroxyl.

As used herein, the term “alkylene” refers to an alkyl group having the specified number of carbons, for example from 2 to 12 carbon atoms, which contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Preferred cycloalkyls have from 3-10 carbon atoms in their ring structure, and more preferably have 3-6 carbons in the ring structure. Cycloalkyl groups may be substituted or unsubstituted.

As used herein, the term “halocycloalkyl” refers to an cycloalkyl group as hereinbefore defined substituted with at least one halogen.

“Cycloheteroalkyl” or “heterocycloalkyl” refers to an cycloalkyl moiety as hereinbefore defined which contain one or more oxygen, sulfur, nitrogen, phosphorus, or silicon atoms in place of carbon atoms. Preferred cycloheteroalkyls have from 4-8 carbon atoms and heteroatoms in their ring structure, and more preferably have 4-6 carbons and heteroatoms in the ring structure. Cycloheteroalkyl or heterocycloalkyl groups may be substituted or unsubstituted.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, arylalkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an arylalkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the definition of each expression, e.g., alkyl, m, n, etc., when it occurs more than once in any structure, is intended to be independent of its definition elsewhere in the same structure.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

For purposes of this invention, the chemical elements are identified in accordance with the Periodic Table of the Elements, CAS version, Handbook of Chemistry and Physics, 67th Ed., 1986-87, inside cover.

### Compounds of the Invention

One aspect of the invention provides a compound of Formula (I):

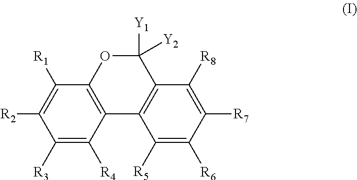

wherein
Y₁ and Y₂ are each alkyl; or taken together with the carbon to which they are bonded combine to form an unsubstituted or substituted spiro cycloalkyl;
R₁, R₄, R₅, and R₈ are independently selected from —H and halogen;
R₂ and R₇ are independently selected from —H, —OH, —OAc, —NH₂, halogen, —CN, —CF₃, —CO₂H, —NO₂, —NHAc, alkyl, haloalkyl, alkenyl, alkynyl, cycloalkyl, heterocycloalkyl, alkylamino, alkyl-R₉, alkenyl-R₉, alkynyl-R₉, —OR₁₀, —NHR₁₀, —NR₁₁C(O)R₁₂, —C(O)NR₁₁R₁₂, and —NR₁₁SO₂R₁₂;
R₃ and R₆ are independently selected from alkyl and cycloalkyl;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from alkyl, hydroxyalkyl, aminoalkyl, alkyl-O-alkyl, alkyl-O-alkyl-OH, alkyl-O-alkyl-O-alkyl, alkenyl, alkynyl, arylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of R₁₁ is selected from H and alkyl; and
each occurrence of R₁₂ is selected from alkyl, alkenyl, alkynyl, cycloalkyl, heterocycloalkyl, O-alkyl, aminoalkyl, arylalkyl, heteroarylalkyl, alkyl-cycloalkyl, and alkyl-heterocycloalkyl;
provided that when R₁, R₄, R₅, and R₈ are each —H, R₂ and R₇ are each —OH, and R₃ and R₆ are each CH₃, then Y₁ and Y₂ are not each -Me or are not taken together with the carbon to which they are bonded to form an unsubstituted spiro cyclobutyl;
or a pharmaceutically acceptable salt thereof.

In certain embodiments, R₃ and R₆ are alkyl.

In certain embodiments, Y₁ and Y₂ are each independently C₁-C₄ alkyl.

In certain embodiments, Y₁ and Y₂ are each —CH₃.

In certain embodiments, Y₁ and Y₂ taken together with the carbon to which they are bonded combine to form an unsubstituted spiro cycloalkyl.

In certain embodiments, Y₁ and Y₂ taken together with the carbon to which they are bonded combine to form an unsubstituted spiro cyclopropyl, cyclobutyl, or cyclopentyl.

In certain embodiments, wherein R₃ and R₆ are each independently C₁-C₄ alkyl.

In certain embodiments, R₃ and R₆ are each independently selected from —CH₃ and —CH₂CH₃.

In certain embodiments, R₃ and R₆ are each —CH₃.

In certain embodiments, R₃ and R₆ are each —CH₂CH₃.

In certain embodiments, one of R₃ and R₆ is —CH₃ and the other of R₃ and R₆ is —CH₂CH₃.

In certain embodiments, R₃ and R₆ are cycloalkyl.

In certain embodiments, R₃ and R₆ are each independently C₃-C₅ cycloalkyl.

In certain embodiments, R₃ and R₆ are each cyclopropyl.

In certain embodiments, one of R₃ and R₆ is C₁-C₄ alkyl and the other of R₃ and R₆ is C₃-C₅ cycloalkyl.

In certain embodiments, one of R₃ and R₆ is —CH₃ and the other of R₃ and R₆ is cyclopropyl.

In certain embodiments, the compound having the structure selected from:

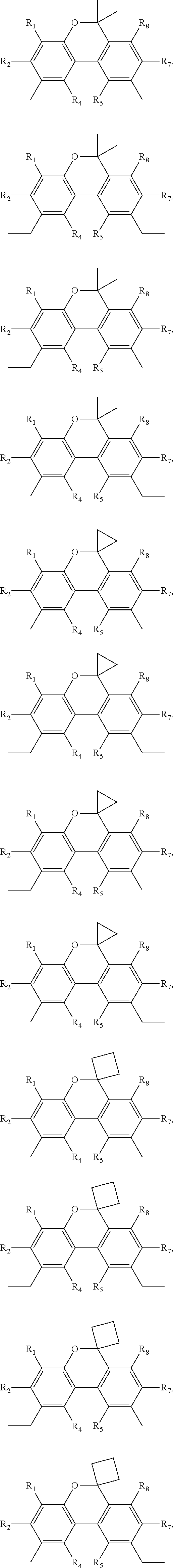

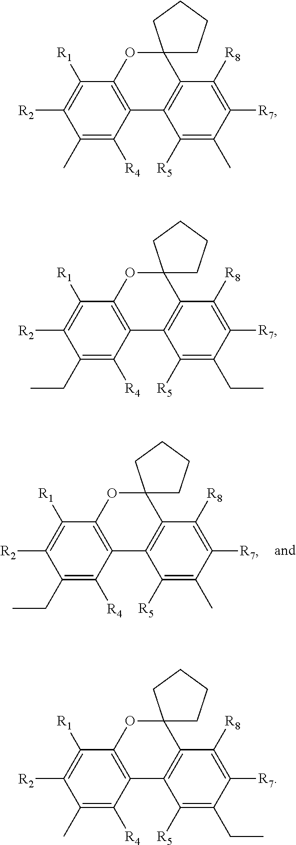

In certain embodiments, the compound having the structure selected from:

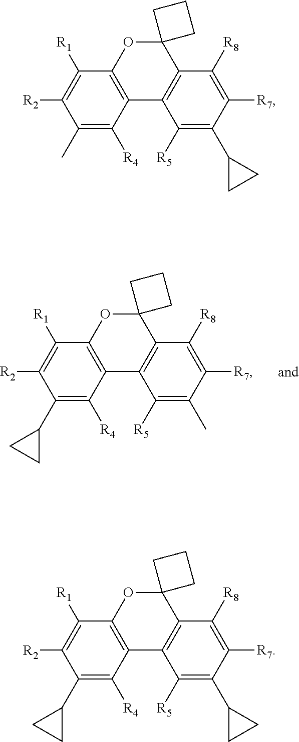

In certain embodiments, R₂ and R₇ are independently selected from —OH, —NH₂, alkylamino, and —OR₁₀.

In certain embodiments, R₂ and R₇ are each OH.

In certain embodiments, R₂ is —OH; and R₇ is —OCH₃.

In certain embodiments, R₇ is —OH; and R₂ is —OCH₃.

In certain embodiments, R₂ is selected from —NH₂, —NHCH₃, and —NH(CH₃)₂; and R₇ is OH.

In certain embodiments, R₇ is selected from —NH₂, —NHCH₃, and —NH(CH₃)₂; and R₂ is OH.

In certain embodiments, R₁, R₄, R₅, and R₈ are each —H.

Another aspect of the invention provides a compound of Formula (II):

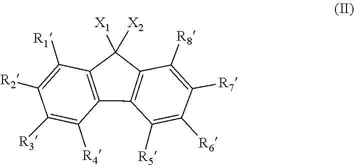

wherein
X₁ and X₂ are each alkyl; or taken together with the carbon to which they are bonded combine to form an unsubstituted or substituted spiro cycloalkyl;
R₁′, R₄′, R₅′, and R₈′ are independently selected from —H, —OH, —NH₂, alkyl, and halogen;
R₂′, R₃′, R₆′, and R₇′ are independently selected from —H, —OH, —OAc, —NH₂, halogen, —CN, —CF₃, —CO₂H, —NO₂, —NHAc, alkyl, haloalkyl, alkenyl, alkynyl, cycloalkyl, heterocycloalkyl, alkylamino, and —OR₈′; and
R₈′ is selected from alkyl, hydroxyalkyl, aminoalkyl, aryl, heteroaryl, cycloalkyl, and heterocycloalkyl;
or a pharmaceutically acceptable salt thereof.

In certain embodiments, X₁ and X₂ are each independently C₁-C₄ alkyl.

In certain embodiments, X₁ and X₂ are each —CH₃.

In certain embodiments, X₁ and X₂ taken together with the carbon to which they are bonded combine to form an unsubstituted spiro cycloalkyl.

In certain embodiments, X₁ and X₂ taken together with the carbon to which they are bonded combine to form an unsubstituted spiro cyclopropyl, cyclobutyl, or cyclopentyl.

In certain embodiments, the compound having the structure selected from:

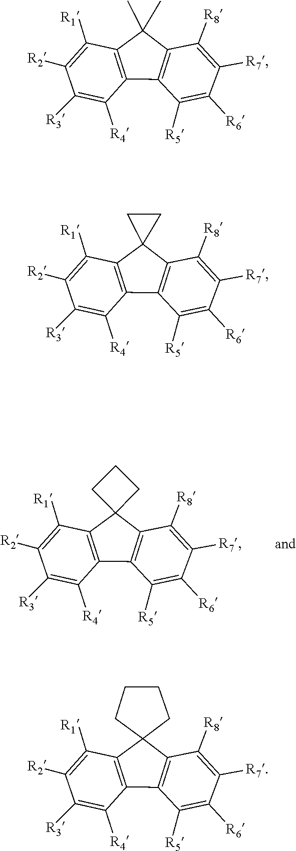

In certain embodiments, R₂′ and R₇′ are independently selected from —OH, —NH₂, alkylamino, and —OR₁₀.

In certain embodiments, R₂′ and R₇′ are each OH.

In certain embodiments, R₂′ is —OH; and R₇′ is —OCH₃.

In certain embodiments, R₇′ is —OH; and R₂′ is —OCH₃.

In certain embodiments, R₂′ is selected from —NH₂, —NHCH₃, and —NH(CH₃)₂; and R₇′ is OH.

In certain embodiments, R₇′ is selected from —NH₂, —NHCH₃, and —NH(CH₃)₂; and R₂′ is OH.

In certain embodiments, R₃′ and R₇′ are each independently —H or C₁-C₄ alkyl.

In certain embodiments, R₃′ and R₇′ are each independently —H or —CH₃.

In certain embodiments, R₁′ and R₈′ are each independently —H or C₁-C₄ alkyl.

In certain embodiments, R₁′ and R₈′ are each independently —H or —CH₃.

In certain embodiments, R₄′ and R₅′ are each independently —H or —OH.

In certain embodiments, the compound is selected from Table 1.

TABLE 1

| Compound # | Structure |
|---|---|
| 1 | 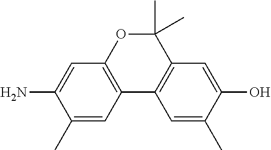 [SMILES: Cc1cc2c(cc1N)OC(C)(C)c1cc(O)c(C)cc1-2] |
| 2 | 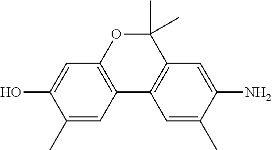 [SMILES: Cc1cc2c(cc1N)C(C)(C)Oc1cc(O)c(C)cc1-2] |
| 2a | 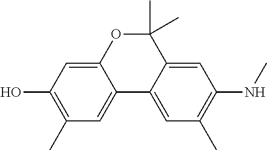 [SMILES: CNc1cc2c(cc1C)-c1cc(C)c(O)cc1OC2(C)C] |
| 3 | 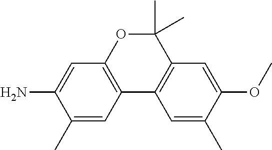 [SMILES: COc1cc2c(cc1C)-c1cc(C)c(N)cc1OC2(C)C] |
| 4 | 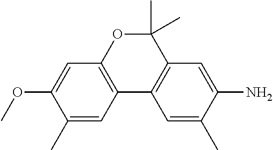 [SMILES: COc1cc2c(cc1C)-c1cc(C)c(N)cc1C(C)(C)O2] |
| 5 | 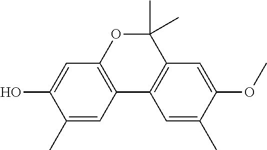 [SMILES: COc1cc2c(cc1C)-c1cc(C)c(O)cc1OC2(C)C] |
| 6 | 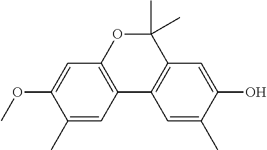 [SMILES: COc1cc2c(cc1C)-c1cc(C)c(O)cc1C(C)(C)O2] |
| 7 | 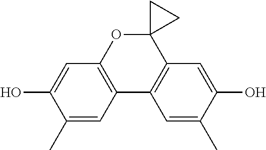 [SMILES: Cc1cc2c(cc1O)OC1(CC1)c1cc(O)c(C)cc1-2] |
| 8 | 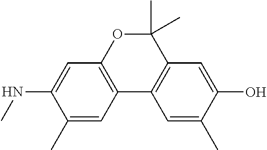 [SMILES: CNc1cc2c(cc1C)-c1cc(C)c(O)cc1C(C)(C)O2] |
| 9 | 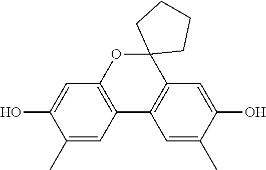 [SMILES: Cc1cc2c(cc1O)OC1(CCCC1)c1cc(O)c(C)cc1-2] |
| 10 | 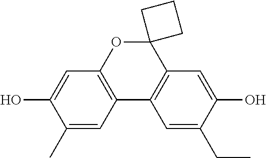 [SMILES: CCc1cc2c(cc1O)C1(CCC1)Oc1cc(O)c(C)cc1-2] |
| 11 | 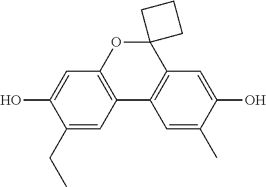 [SMILES: CCc1cc2c(cc1O)OC1(CCC1)c1cc(O)c(C)cc1-2] |
| 12 | 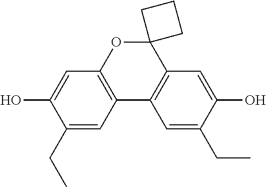 [SMILES: CCc1cc2c(cc1O)OC1(CCC1)c1cc(O)c(CC)cc1-2] |
| 17 | 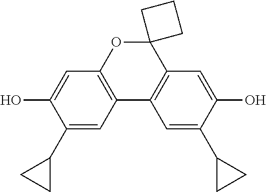 [SMILES: Oc1cc2c(cc1C1CC1)-c1cc(C3CC3)c(O)cc1C1(CCC1)O2] |
| 18 | 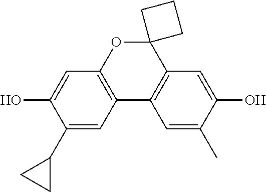 [SMILES: Cc1cc2c(cc1O)C1(CCC1)Oc1cc(O)c(C3CC3)cc1-2] |
| 19 | 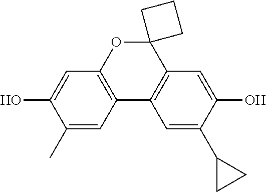 [SMILES: Cc1cc2c(cc1O)OC1(CCC1)c1cc(O)c(C3CC3)cc1-2] |

In certain embodiments, the compound is selected from Table 2.

TABLE 2

| Compound # | Structure |
|---|---|
| 13 | 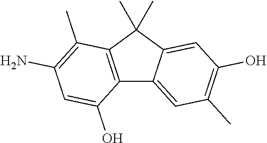 [SMILES: Cc1cc2c(cc1O)C(C)(C)c1c(C)c(N)cc(O)c1-2] |
| 14 | 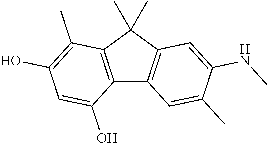 [SMILES: CNc1cc2c(cc1C)-c1c(O)cc(O)c(C)c1C2(C)C] |
| 14a | 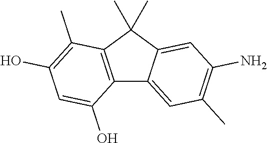 [SMILES: Cc1cc2c(cc1N)C(C)(C)c1c(C)c(O)cc(O)c1-2] |
| 15 | 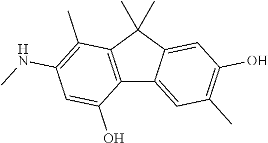 [SMILES: CNc1cc(O)c2c(c1C)C(C)(C)c1cc(O)c(C)cc1-2] |
| 16 | 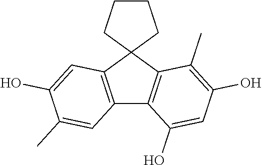 [SMILES: Cc1cc2c(cc1O)C1(CCCC1)c1c(C)c(O)cc(O)c1-2] |

In some embodiments, the compounds are atropisomers. Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. Such compounds are useful, for example, as analytical tools, as probes in biological assays, or as therapeutic agents in accordance with the present invention. For example, in the case of variable R¹, the (C1-C₄)alkyl or the —O—(C1-C₄)alkyl can be suitably deuterated (e.g., —CD₃, —OCD₃).

Any compound of the invention can also be radiolabed for the preparation of a radiopharmaceutical agent.

### Methods of Treatment

One aspect of the invention relates to a method of inhibiting ferroptosis, comprising administering to a subject in need thereof an effective amount of a compound of Formula (I) or (II).

Another aspect of the invention relates to a method of treating an inflammatory disease, neuronal disease, or neurodegenerative disease at least partially mediated by ferroptosis, comprising administering to a subject in need thereof an effective amount of a compound of Formula (I) or (II).

Another aspect of the invention relates to a method of treating an inflammatory disease at least partially mediated by ferroptosis, comprising administering to a subject in need thereof an effective amount of a compound of Formula (I) or (II).

Another aspect of the invention relates to a method of treating a neuronal disease at least partially mediated by ferroptosis, comprising administering to a subject in need thereof an effective amount of a compound of Formula (I) or (II).

Another aspect of the invention relates to a method of treating a neurodegenerative disease at least partially mediated by ferroptosis, comprising administering to a subject in need thereof an effective amount of a compound of Formula (I) or (II).

A further aspect of the invention relates to a method of treating a mitochondrial disease in a subject in need thereof, comprising administering to the subject an effective amount of a compound of Formula (I) or (II).

In one embodiment, the mitochondrial disease affects the muscle of the subject, e.g. mitochondrial myopathies. In another embodiment, the mitochondrial disease affects the eye of the subject, e.g. external progressive ophtalmoplegia. In other embodiments, the mitochondrial disease is Alper's disease, Barth syndrome, beta-oxidation defects, carnitine deficiency, carnitine-acyl-carnitine deficiency, chronic progressive external ophthalmoplegia syndrome, or co-enzyme Q10 deficiency.

In one aspect, the invention relates to methods of treating a muscle or a neuromuscular disease in a subject in need thereof comprising administering to the subject an effective amount of the compound of any one of Formulas (I) or (II). In one embodiment, the muscle or neuromuscular disease is sarcopenia. In another embodiment, the muscle or neuromuscular disease is a muscular dystrophy. In another embodiment, the muscle or neuromuscular disease is a myopathy. In another embodiment, the muscle or neuromuscular disease is Duchenne muscular dystrophy. In another embodiment, the muscle or neuromuscular disease is inclusion body myositis (IBM) or sporadic inclusion body myositis (sIBM). In another embodiment, the muscle or neuromuscular disease is selected from mitochondrial myopathies. In other embodiments, the muscle or neuromuscular disease is muscle aging and weakness, frailty, sarcopenia, mitochondrial myopathies, or muscle rhabdomyolysis.

In one aspect, the invention relates to methods of treating a neuronal or neurodegenerative disease in a subject in need thereof comprising administering to the subject an effective amount of the compound of any one of Formulas (I) or (II). In some embodiments, the neuronal or neurodegenerative disease is Alzheimer's disease, Parkinson's disease, Huntington's disease, amyotrophic lateral sclerosis (also known as ALS and as Lou Gehrig's disease), as well as AIDS dementia complex, adrenoleukodystrophy, Alexander disease, Alper's disease, ataxia telangiectasia, Batten disease, bovine spongiform encephalopathy (BSE), Canavan disease, corticobasal degeneration, Creutzfeldt-Jakob disease, dementia with Lewy bodies, fatal familial insomnia, frontotemporal lobar degeneration, Kennedy's disease, Krabbe disease, Lyme disease, Machado-Joseph disease, multiple sclerosis, multiple system atrophy, neuroacanthocytosis, Niemann-Pick disease, Pick's disease, primary lateral sclerosis, progressive supranuclear palsy, Refsum disease, Sandhoff disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, and wobbly hedgehog syndrome.

In one aspect, the invention relates to methods of treating ischemia-reperfusion injury in a subject in need thereof comprising administering to the subject an effective amount of the compound of any one of Formulas (I) or (II).

### Pharmaceutical Compositions, Routes of Administration, and Dosing

In certain embodiments, the invention is directed to a pharmaceutical composition, comprising a compound of the invention and a pharmaceutically acceptable carrier. In certain embodiments, the invention is directed to a pharmaceutical composition, comprising the compound of any one of Formulas (I) or (II) and a pharmaceutically acceptable carrier. In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of ischemia-reperfusion injury.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### Examples

The invention is further described in the following examples, which do not limit the scope of the invention described in the claims.

### Example 1: Synthesis of Representative Compounds of the Invention

All reactions were performed with oven-dried glassware and under an inert atmosphere (nitrogen) unless otherwise stated. All solvents were used as purchased unless otherwise stated. Commercial reagents were used as purchased without further purification. Organic solutions were concentrated under reduced pressure on a Büchi rotary evaporator.

Thin-layer chromatography was carried out using Merck Kieselgel 60 F254 (230-400 mesh) fluorescent treated silica and were visualized under UV light (254 and 366 nm) and/or by staining with aqueous potassium permanganate solution. 1H NMR spectra were recorded in deuterated solvents on Bruker spectrometer at 400 MHz or Nanalysis NMReady-60PRO spectrometer at 60 MHz, with residual protic solvent as the internal standard. 13C NMR spectra were recorded in deuterated solvents on Bruker spectrometer at 100 MHz, with the central peak of the deuterated solvent as the internal standard. Chemical shifts (6) are given in parts per million (ppm) and coupling constants (J) are given in Hertz (Hz) rounded to the nearest 0.1 Hz. The 1H NMR spectra are reported as δ/ppm downfield from tetramethylsilane (multiplicity, number of protons, coupling constant J/Hz). The ¹³C NMR spectra are reported as δ/ppm. TLC-MS data was obtained on Advion Expression CMS coupled with Plate Express TLC-plate Reader. Medium pressure liquid chromatography (MPLC) was performed on a Biotage Isolera Four with built-in UV-detector and fraction collector with Interchim silica gel columns.

1. Synthesis of ((8-(benzyloxy)-2,6,6,9-tetramethyl-6H-benzo[c]chromen-3-yl)oxy)(tert-butyl)dimethylsilane

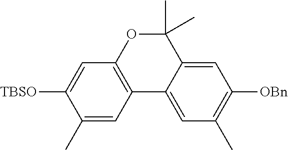 [SMILES: Cc1cc2c(cc1OCc1ccccc1)C(C)(C)Oc1cc(O[Si](C)(C)C(C)(C)C)c(C)cc1-2]

((8-(benzyloxy)-2,6,6,9-tetramethyl-6H-benzo[c]chromen-3-yl)oxy)(tert-butyl)dimethylsilane was prepared in 7 steps from 3-hydroxy-4-methylbenzoic acid

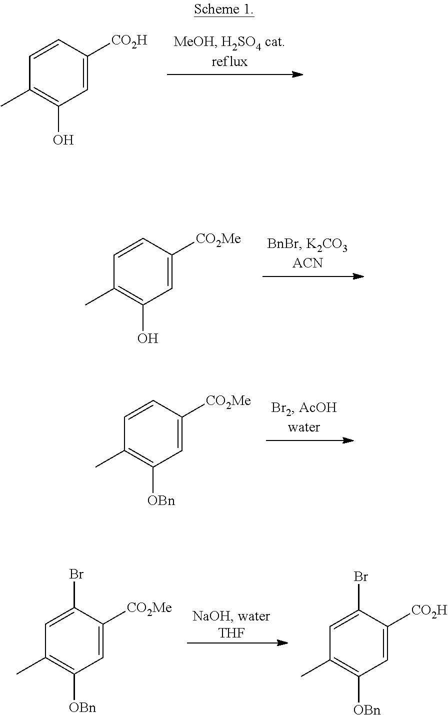 [SMILES: Cc1ccc(C(=O)O)cc1O ; COC(=O)c1ccc(C)c(O)c1 ; COC(=O)c1ccc(C)c(OCc2ccccc2)c1 ; COC(=O)c1cc(OCc2ccccc2)c(C)cc1Br ; Cc1cc(Br)c(C(=O)O)cc1OCc1ccccc1]

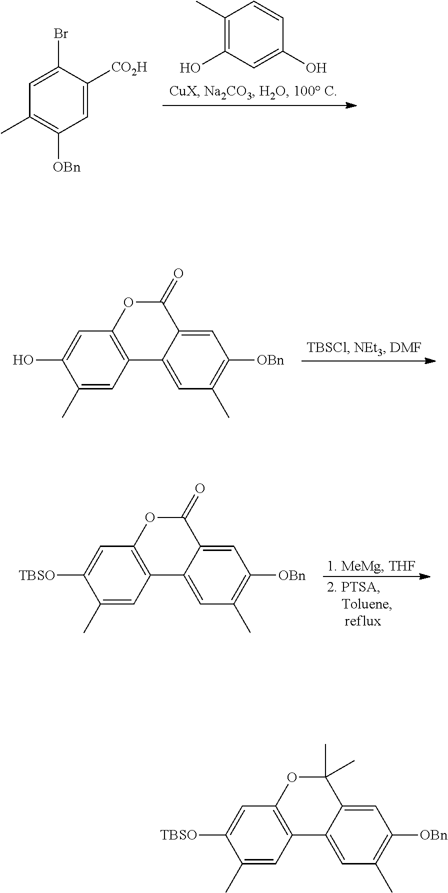

### Step 1: Synthesis of methyl 3-hydroxy-4-methylbenzoate

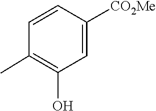

sulfuric acid (1.3 g, 0.70 mL, 0.2 Eq, 13 mmol) was added to a suspension of 3-hydroxy-4-methylbenzoic acid (10 g, 1 Eq, 66 mmol) in methanol (2.1 g, 0.16 L, 0.4 molar, 1 Eq, 66 mmol) and the mixture was refluxed o.n. Methanol was evaporated under vacuum and the crude was extracted with EtOAc and Na2CO3 saturated solution. The organic phase was washed with water, dried over sodium sulfate and concentrated under vacuum to give methyl 3-hydroxy-4-methylbenzoate (8.7 g, 52 mmol, 80%) as a white solid. ¹H NMR (400 MHz, DMSO) δ 9.73 (d, J=0.5 Hz, 1H), 7.39 (d, J=1.7 Hz, 1H), 7.31 (dd, J=7.7, 1.7 Hz, 1H), 7.19 (d, J=7.7 Hz, 1H), 3.80 (s, 3H), 2.17 (d, J=0.6 Hz, 3H).

### Step 2: synthesis of methyl 3-(benzyloxy)-4-methylbenzoate CO₂Me

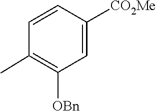 [SMILES: COC(=O)c1ccc(C)c(OCc2ccccc2)c1]

methyl 3-hydroxy-4-methylbenzoate (8.7 g, 1 Eq, 52 mmol) was dissolved in acetonitrile (2.1 g, 0.13 L, 0.4 molar, 1 Eq, 52 mmol). potassium carbonate (7.2 g, 1 Eq, 52 mmol) was added followed by benzyl bromide (9.0 g, 6.2 mL, 1 Eq, 52 mmol) and the mixture was heated at 50° C. o.n. Water was added and the aqueous phase was extracted with EtOAc. The organic phase was washed successively with water and brine, dried over sodium sulfate and evaporated under vacuum to give methyl 3-(benzyloxy)-4-methylbenzoate (10.9 g, 42.5 mmol, 81%) as a yellowish solid. ¹H NMR (400 MHz, CDCl₃) δ 7.61-7.57 (m, 2H), 7.51-7.30 (m, 6H), 7.23-7.19 (m, 1H), 5.14 (s, 2H), 3.91 (s, 3H), 2.33 (d, J=0.7 Hz, 3H).

### Step 3: Synthesis of methyl 5-(benzyloxy)-2-bromo-4-methylbenzoate

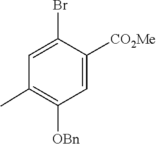 [SMILES: COC(=O)c1cc(OCc2ccccc2)c(C)cc1Br]

bromine (8.16 g, 2.63 mL, 1.2 Eq, 51.0 mmol) was added to a RT suspension of methyl methyl 3-(benzyloxy)-4-methylbenzoate (10.9 g, 1 Eq, 42.5 mmol) in acetic acid (51.1 g, 48.7 mL, 20 Eq, 851 mmol) and water (38.3 g, 38.3 mL, 50 Eq, 2.13 mol) and the resulting mixture was heated to 60° C. overnight. After cooling to room temperature, Ice was added and the reaction mixture was stirred at room temperature for 2 h. The precipitate was filtered off and rinsed with cold water, dried under vacuum to give methyl 5-(benzyloxy)-2-bromo-4-methylbenzoate (13 g, 39 mmol, 91%) as a white solid. ¹H NMR (400 MHz, CDCl₃) δ 7.46-7.32 (m, 7H), 5.09 (s, 2H), 3.92 (s, 3H), 2.27 (d, J=0.8 Hz, 3H).

### Step 4: Synthesis of 5-(benzyloxy)-2-bromo-4-methylbenzoic acid

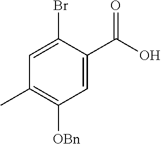 [SMILES: Cc1cc(Br)c(C(=O)O)cc1OCc1ccccc1]

methyl 5-(benzyloxy)-2-bromo-4-methylbenzoate (3000.00 mg, 1 Eq, 8.950 mmol) was dissolved in methanol (286.8 mg, 17.90 mL, 0.5 molar, 1 Eq, 8.950 mmol), THF (645.4 mg, 44.75 mL, 0.2 molar, 1 Eq, 8.950 mmol) at room temperature. then cooled down to 0° C. A solution of LiOH (643.0 mg, 3 Eq, 26.85 mmol) in water (161.3 mg, 17.90 mL, 0.5 molar, 1 Eq, 8.950 mmol) was added dropwise and stirring continued overnight. Methanol and THF were evaporated under vacuum. The crude obtained was extracted with ethyl acetate and HCl 1M twice. The combined organic phases were washed with brine once, dried over sodium sulfate and concentrated under vacuum to give 5-(benzyloxy)-2-bromo-4-methylbenzoic acid (2.7 g, 8.4 mmol, 94%) as a white solid. ¹H NMR (400 MHz, DMSO) δ 13.26 (s, 1H), 7.52 (d, J=0.9 Hz, 1H), 7.48-7.44 (m, 2H), 7.43-7.38 (m, 3H), 7.36-7.31 (m, 1H), 5.17 (s, 2H), 2.26-2.19 (m, 3H).

### Step 5: Synthesis of 8-(benzyloxy)-3-hydroxy-2,9-dimethyl-6H-benzo[c]chromen-6-one

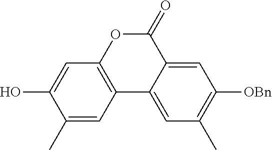 [SMILES: Cc1cc2c(cc1O)oc(=O)c1cc(OCc3ccccc3)c(C)cc12]

sodium carbonate (2.970 g, 3.0 Eq, 28.02 mmol) was dissolved in water (168.3 mg, 46.70 mL, 0.2 molar, 1 Eq, 9.341 mmol) at room temperature. 4-methylbenzene-1,3-diol (2.319 g, 2.0 Eq, 18.68 mmol) was added portionwise and heated at 60° C. for 30 minutes. 5-(benzyloxy)-2-bromo-4-methylbenzoic acid (3000 mg, 1 Eq, 9.341 mmol) was added portionwise and stirring continued T 60° C. for 1 h. CuI (1.245 g, 0.7 Eq, 6.539 mmol) was added and the reaction mixture was heated at the same temperature overnight. A precipitate was formed after addition of CuI. The precipitate was filtered of and washed successively with water and HCl 1M. the solid was dried under high vacuum to give 8-(benzyloxy)-3-hydroxy-2,9-dimethyl-6H-benzo[c]chromen-6-one (1.8 g, 5.2 mmol, 56%) as a beige solid. ¹H NMR (400 MHz, DMSO) δ 10.16 (s, 1H), 8.12 (s, 1H), 7.97 (s, 1H), 7.63 (s, 1H), 7.51 (d, J=7.0 Hz, 2H), 7.42 (t, J=7.4 Hz, 2H), 7.36-7.32 (m, 1H), 6.74 (s, 1H), 5.26 (s, 2H), 2.39 (s, 3H), 2.21 (s, 3H).

### Step 6: synthesis 8-(benzyloxy)-3-((tert-butyldimethylsilyl)oxy)-2,9-dimethyl-6H-benzo[c]chromen-6-one

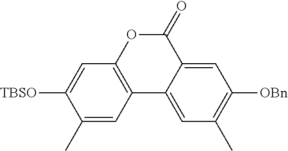 [SMILES: Cc1cc2c(cc1OCc1ccccc1)c(=O)oc1cc(O[Si](C)(C)C(C)(C)C)c(C)cc12]

8-(benzyloxy)-3-hydroxy-2,9-dimethyl-6H-benzo[c]chromen-6-one (1.7 g, 1 Eq, 4.9 mmol) was dissolved in DMF (0.36 g, 49 mL, 0.1 molar, 1 Eq, 4.9 mmol) at room temperature. triethylamine (1.2 g, 1.7 mL, 2.5 Eq, 12 mmol) was added and the mixture was cooled down to 0° C. TBDMS-Cl (0.89 g, 1.2 Eq, 5.9 mmol) was added and stirring continued at r.t. o.n. Reaction mixture was extracted with HCl 1M and EtOAc. The combined organic phases were washed with successively with water and brine, dried over sodium sulfate and concentrated under vacuum. The crude material was purified by flash column chromatography on silica (0-50% EtOAc in Hex) to afford 8-(benzyloxy)-3-((tert-butyldimethylsilyl)oxy)-2,9-dimethyl-6H-benzo[c]chromen-6-one (1.18 g, 2.56 mmol, 52%). R_{f}=0.7 (EtOAc/Cyclohexane 20%). ¹H NMR (400 MHz, CDCl₃) δ 7.80 (d, J=1.0 Hz, 1H), 7.78 (s, 1H), 7.73 (s, 1H), 7.51-7.47 (m, 2H), 7.45-7.39 (m, 2H), 7.38-7.33 (m, 1H), 6.79 (s, 1H), 5.20 (s, 2H), 2.46 (d, J=0.8 Hz, 3H), 2.30 (d, J=0.7 Hz, 3H), 1.04 (s, 9H), 0.28 (s, 6H).

### Step 7: synthesis ((8-(benzyloxy)-2,6,6,9-tetramethyl-6H-benzo[c]chromen-3-yl)oxy)(tert-butyl)dimethylsilane

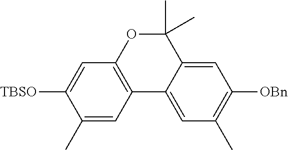 [SMILES: Cc1cc2c(cc1OCc1ccccc1)C(C)(C)Oc1cc(O[Si](C)(C)C(C)(C)C)c(C)cc1-2]

8-(benzyloxy)-3-((tert-butyldimethylsilyl)oxy)-2,9-dimethyl-6H-benzo[c]chromen-6-one (1170 mg, 1 Eq, 2.540 mmol) was dissolved in THF (183.2 mg, 25.40 mL, 0.1 molar, 1 Eq, 2.540 mmol) and the reaction was cooled to 0° C. in an ice-bath. Following methylmagnesium bromide (1.333 g, 3.725 mL, 3 molar, 4.4 Eq, 11.18 mmol) was added in one portion. The reaction was stirred at 0° C. for 10 min before being allowed to warm to room temperature. Stirring at room temperature was continued for 1 h before the reaction was quenched with water and extracted with EtOAc (3×) The combined organic layers were dried over anhydrous Na₂SO₄, filtered and concentrated under vacuo. The crude was dissolved in EtOAc 20 mL, PTSOH (48.31 mg, 0.1 Eq, 254.0 μmol) was added in one portion and the mixture was heated at 60° C. for 1 h. The reaction mixture was extracted with EtOAc and sodium bicarbonate saturated solution once, dried over sodium sulfate and concentrated under vacuum to give ((8-(benzyloxy)-2,6,6,9-tetramethyl-6H-benzo[c]chromen-3-yl)oxy)(tert-butyl)dimethylsilane (1100 mg, 2.317 mmol, 91.23%) as brownish solid. R_{f}=0.6 (EtOAc/Cyclohexane 20%). ¹H NMR (400 MHz, CDCl₃) δ 7.57-7.46 (m, 2H), 7.45-7.38 (m, 2H), 7.37-7.30 (m, 1H), 7.21 (s, 1H), 6.85 (d, J=0.9 Hz, 1H), 6.44 (s, 1H), 5.14 (s, 2H), 2.24 (d, J=0.8 Hz, 3H), 2.14 (s, 3H), 1.51 (s, 3H), 1.40 (s, 3H), 1.03 (s, 9H), 0.26 (s, 3H), 0.25 (s, 3H).

### 2. Synthesis of 2,6,6,9-tetramethyl-8-(methylamino)-6H-benzo[c]chromen-3-ol (2a)

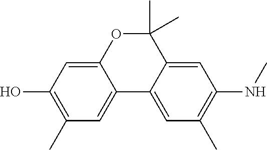 [SMILES: CNc1cc2c(cc1C)-c1cc(C)c(O)cc1OC2(C)C]

2a was synthesized in 4 steps starting from ((8-(benzyloxy)-2,6,6,9-tetramethyl-6H-benzo[c]chromen-3-yl)oxy)(tert-butyl)dimethylsilane.

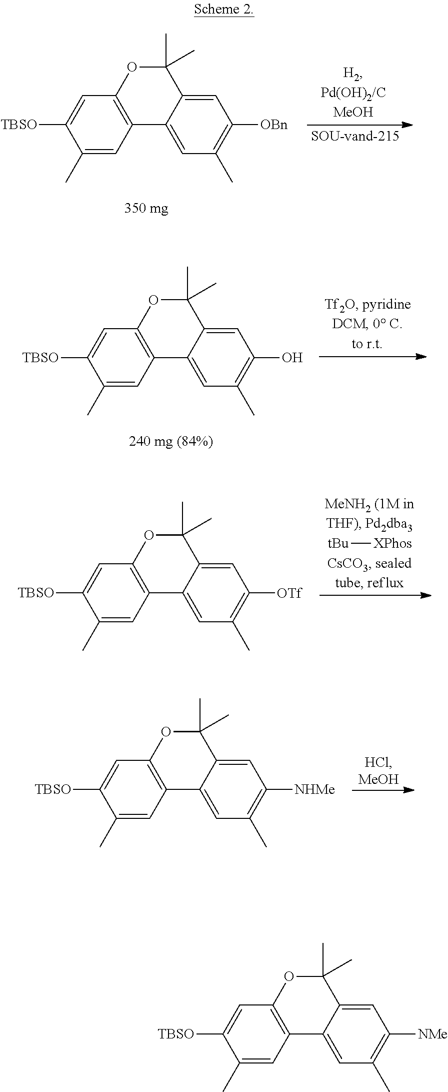

### Step 1: synthesis of 3-((tert-butyldimethylsilyl)oxy)-2,6,6,9-tetramethyl-6H-benzo[c]chromen-8-ol

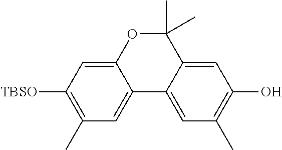 [SMILES: Cc1cc2c(cc1O)C(C)(C)Oc1cc(O[Si](C)(C)C(C)(C)C)c(C)cc1-2]

((8-(benzyloxy)-2,6,6,9-tetramethyl-6H-benzo[c]chromen-3-yl)oxy)(tert-butyl)dimethylsilane (400 mg, 1 Eq, 843 μmol) was dissolved in methanol (27.0 mg, 8.43 mL, 0.1 molar, 1 Eq, 843 μmol) and dichloromethane (71.6 mg, 4.21 mL, 0.2 molar, 1 Eq, 843 μmol). palladium hydroxide on carbon (118 mg, 20% Wt, 0.2 Eq, 169 μmol) was added in one portion and the suspension was hydrogenated under atmospheric pressure overnight. After completion, the reaction mixture was filtered over a pad of celite. p-Toluenesulfonicacidmonohydrate (16.0 mg, 12.9 μL, 0.1 Eq, 84.3 μmol) was added then the mixture was concentrated and loaded on silica to be purified by flash column chromatography (SiO₂, 12 g, MeOH in DCM 0-5% to give 3-((tert-butyldimethylsilyl)oxy)-2,6,6,9-tetramethyl-6H-benzo[c]chromen-8-ol (150 mg, 390 μmol, 46.3%) as a white solid. ¹H NMR (400 MHz, DMSO) δ 9.35 (s, 1H), 7.49 (s, 1H), 7.44 (s, 1H), 6.67 (s, 1H), 6.26 (s, 1H), 2.15 (s, 3H), 2.13 (s, 3H), 1.47 (s, 6H), 0.98 (s, 9H), 0.20 (s, 6H).

### Step 2: synthesis of 3 3-((tert-butyldimethylsilyl)oxy)-2,6,6,9-tetramethyl-6H-benzo[c]chromen-8-yl trifluoromethanesulfonate

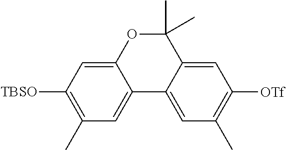 [SMILES: Cc1cc2c(cc1O[Si](C)(C)C(C)(C)C)OC(C)(C)c1cc(OS(=O)(=O)C(F)(F)F)c(C)cc1-2]

triflicanhydride (165 mg, 98.8 μL, 1.5 Eq, 585 μmol) was added dropwise to a solution of 3-((tert-butyldimethylsilyl)oxy)-2,6,6,9-tetramethyl-6H-benzo[c]chromen-8-ol (150 mg, 1 Eq, 390 μmol) and pyridine (309 mg, 315 μL, 10 Eq, 3.90 mmol) at 0° C. in DCM 10 mL and the mixture was stirred for 3 h at room temperature. The reaction was monitored by TLC eluent EtOAc/cyclohexane 10%. Dichloromethane was evaporated under vacuum and the crude was extracted with HCl 1M and EtOAc. The organic phase was washed with water once, dried over sodium sulfate and concentrated under vacuum to give 3-((tert-butyldimethylsilyl)oxy)-2,6,6,9-tetramethyl-6H-benzo[c]chromen-8-yl trifluoromethanesulfonate (140 mg, 271 μmol, 69.5%) used to the next step without further purification. R_{f}0.6 (EtOAc/Cyclohexane 40%).

### Step 3: synthesis of 3-((tert-butyldimethylsilyl)oxy)-N,2,6,6,9-pentamethyl-6H-benzo[c]chromen-8-amine

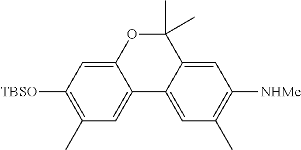 [SMILES: CNc1cc2c(cc1C)-c1cc(C)c(O[Si](C)(C)C(C)(C)C)cc1OC2(C)C]

t-Bu XPhos (23 mg, 0.4 Eq, 54 μmol) was added to a suspension of Tris(dibezylideneacetone)dipalladium (25 mg, 0.2 Eq, 27 μmol) in dioxane 8 mL and the mixture was stirred 5 minute at rt. 3-((tert-butyldimethylsilyl)oxy)-2,6,6,9-tetramethyl-6H-benzo[c]chromen-8-yl trifluoromethanesulfonate (70 mg, 1 Eq, 0.14 mmol), methanamine (63 mg, 1.0 mL, 2 molar, 15 Eq, 2.0 mmol) and cesium carbonate (427 mg, 1.31 mmol) were successively added and the reaction mixture was refluxed for 3 hours. Water was added and the mixture extracted with EA 3 times. The combined organic phases were dried over sodium sulfate, filtered and concentrated under vacuum. The crude was loaded on silica and purified by FC eluent EA/cylohexane 0% to 5% to 10% 20% to give 3-((tert-butyldimethylsilyl)oxy)-N,2,6,6,9-pentamethyl-6H-benzo[c]chromen-8-amine (40 mg, 0.10 mmol, 74%) as yellowish oil. Product contaminated with ligand. R_{f} 0.5 (EtOAc/Cyclohexane 20%). MS (ESI+): m/z=397.24 found 398.3.

### Step 4: synthesis of 2,6,6,9-tetramethyl-8-(methylamino)-6H-benzo[c]chromen-3-ol (2a)

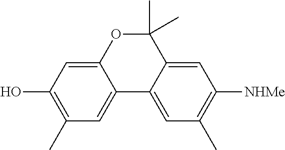 [SMILES: CNc1cc2c(cc1C)-c1cc(C)c(O)cc1OC2(C)C]

3-((tert-butyldimethylsilyl)oxy)-N,2,6,6,9-pentamethyl-6H-benzo[c]chromen-8-amine (40 mg, 1 Eq, 0.10 mmol) was dissolved in dry methanol (3.2 mg, 2.0 mL, 0.05 molar, 1 Eq, 0.10 mmol)hydrogen chloride Methanol solution (34 mg, 0.40 mL, 1.25 molar, 5 Eq, 0.50 mmol) was added at r.t. and the mixture was stirred o.n. Solvent was evaporated and the crude was loaded on silica purified By FC: eluent MeOH/DCM 0% to 20% to give 2,6,6,9-tetramethyl-8-(methylamino)-6H-benzo[c]chromen-3-ol (10 mg, 35 μmol, 35%). R_{f}0.5 (MeOH/DCM 10%) ¹H NMR (400 MHz, DMSO) δ 9.17 (s, 1H), 7.32 (s, 1H), 7.29 (s, 1H), 6.32 (s, 1H), 6.28 (s, 1H), 2.75 (s, 3H), 2.10 (s, 3H), 2.08 (s, 3H), 1.48 (s, 6H).

### 3. Synthesis of 8-amino-2,6,6,9-tetramethyl-6H-benzo[c]chromen-3-ol (2)

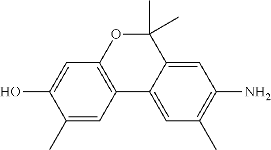 [SMILES: Cc1cc2c(cc1N)C(C)(C)Oc1cc(O)c(C)cc1-2]

2 is prepared in 2 steps from 3 3-((tert-butyldimethylsilyl)oxy)-2,6,6,9-tetramethyl-6H-benzo[c]chromen-8-yl trifluoromethanesulfonate

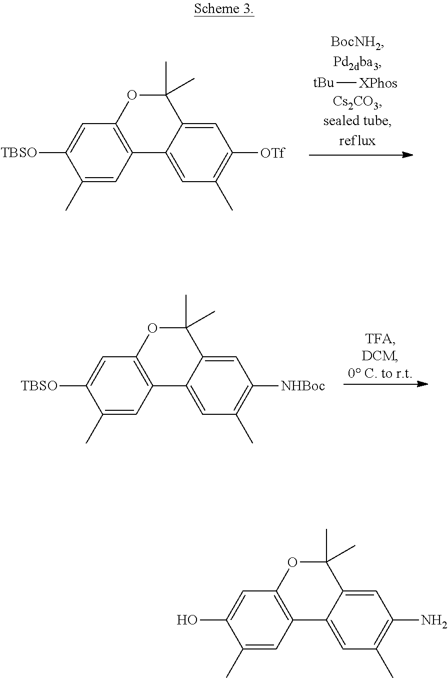

Step 1: synthesis of tert-butyl (3-((tert-butyldimethylsilyl)oxy)-2,6,6,9-tetramethyl-6H-benzo[c]chromen-8-yl)carbamate

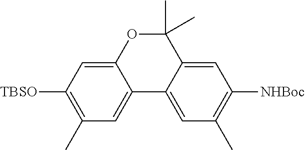 [SMILES: Cc1cc2c(cc1NC(=O)OC(C)(C)C)C(C)(C)Oc1cc(O[Si](C)(C)C(C)(C)C)c(C)cc1-2]

t-Bu XPhos (29.6 mg, 0.2 Eq, 69.7 μmol) was added to a suspension of Tris(dibezylideneacetone)dipalladium (31.9 mg, 0.1 Eq, 34.8 μmol) in dioxane 8 ml and the mixture was stirred 5 minute at rt. 3-((tert-butyldimethylsilyl)oxy)-2,6,6,9-tetramethyl-6H-benzo[c]chromen-8-yl trifluoromethanesulfonate (180 mg, 1 Eq, 348 μmol), Tert-butryl carbamate (163 mg, 4 Eq, 1.39 mmol) and cesium carbonate (427 mg, 1.31 mmol) were added and the reaction mixture was refluxed for 1 hours. Water was added and the mixture extracted with EA 3 times. The combined organic phases were dried over sodium sulfate, filtered and concentrated under vacuum and the crude was loaded on silica and purified by FC eluent EA/cylohexane 0% to 20% to give tert-butyl (3-((tert-butyldimethylsilyl)oxy)-2,6,6,9-tetramethyl-6H-benzo[c]chromen-8-yl)carbamate (70 mg, 0.14 mmol, 42%) as a yellowish solid. R_{f} 0.5 (EtOAc/Cyclohexane 20%) ¹H NMR (400 MHz, CDCl₃) δ 7.72 (s, 1H), 7.39 (d, J=1.1 Hz, 2H), 6.39 (s, 1H), 6.27 (s, 1H), 2.28 (s, 3H), 2.20 (s, 3H), 1.61 (s, 6H), 1.55 (s, 9H), 1.01 (s, 9H), 0.24 (s, 6H).

### Step 2: Synthesis of 8-amino-2,6,6,9-tetramethyl-6H-benzo[c]chromen-3-ol (2)

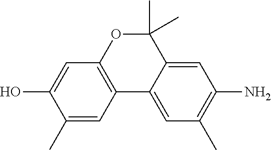 [SMILES: Cc1cc2c(cc1N)C(C)(C)Oc1cc(O)c(C)cc1-2]

tert-butyl (3-((tert-butyldimethylsilyl)oxy)-2,6,6,9-tetramethyl-6H-benzo[c]chromen-8-yl) carbamate (20 mg, 1 Eq, 41 μmol) was dissolved in DCM (2 mL) and cooled down to 0° C. TFA (0.28 g, 0.19 mL, 60 Eq, 2.5 mmol) was added dropwise and stirring continued o.n. The reaction mixture was extracted with EtOAc and NaHCO₃ saturated solution twice. The combined organic phases were dried over sodium sulfate, filtered and concentrated under vacuum and the crude was loaded on silica and purified by FC eluent MeOH/DCM 0% to 5% to 10% 20% to give 8-amino-2,6,6,9-tetramethyl-6H-benzo[c]chromen-3-ol (10 mg, 37 μmol, 90%) as a whit solid.

¹H NMR (400 MHz, DMSO) δ 9.18 (s, 1H), 7.31 (s, 1H), 7.25 (s, 1H), 6.52 (s, 1H), 6.27 (s, 1H), 2.08 (s, 6H), 1.44 (s, 6H).

### 4. Synthesis of 3-amino-2,6,6,9-tetramethyl-6H-benzo[c]chromen-8-ol (1)

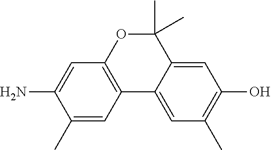 [SMILES: Cc1cc2c(cc1N)OC(C)(C)c1cc(O)c(C)cc1-2]

1_is prepared in 5 steps from ((8-(benzyloxy)-2,6,6,9-tetramethyl-6H-benzo[c]chromen-3-yl)oxy)(tert-butyl)dimethylsilane

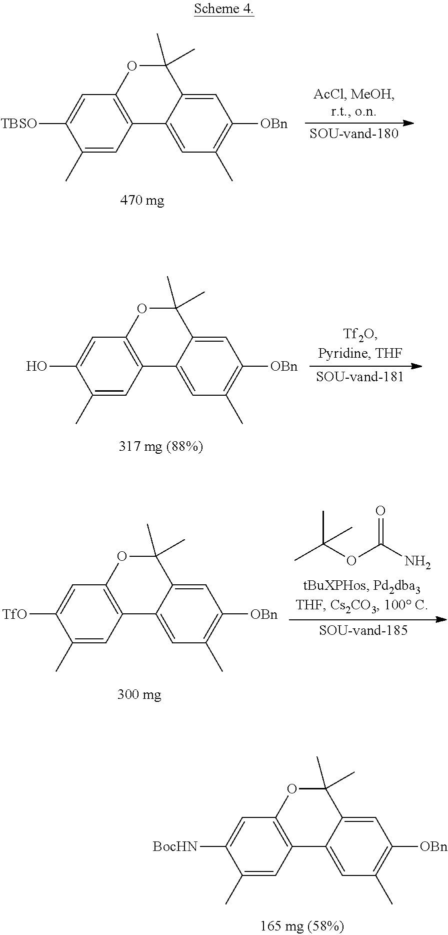

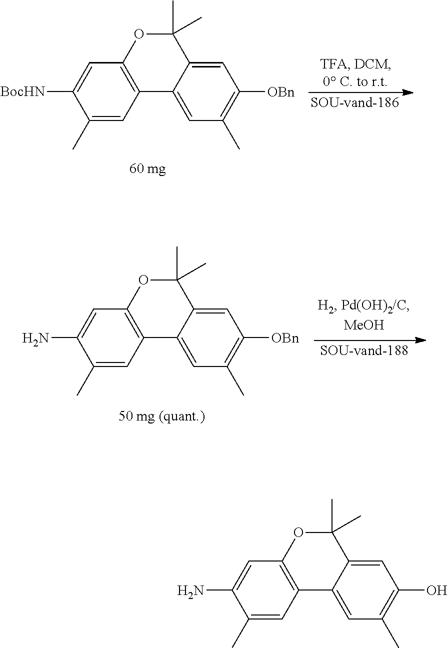 [SMILES: Cc1cc2c(cc1NC(=O)OC(C)(C)C)OC(C)(C)c1cc(OCc3ccccc3)c(C)cc1-2 ; Cc1cc2c(cc1N)OC(C)(C)c1cc(OCc3ccccc3)c(C)cc1-2 ; Cc1cc2c(cc1N)OC(C)(C)c1cc(O)c(C)cc1-2]

### Step 1: Synthesis 8-(benzyloxy)-2,6,6,9-tetramethyl-6H-benzo[c]chromen-3-ol

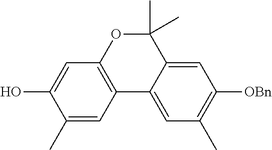 [SMILES: Cc1cc2c(cc1O)OC(C)(C)c1cc(OCc3ccccc3)c(C)cc1-2]

((8-(benzyloxy)-2,6,6,9-tetramethyl-6H-benzo[c]chromen-3-yl)oxy)(tert-butyl)dimethylsilane (470 mg, 1 Eq, 990 μmol) dissolved in methanol dry (31.7 mg, 9.90 mL, 0.1 molar, 1 Eq, 990 μmol) and DCM (84.1 mg, 4.95 mL, 0.2 molar, 1 Eq, 990 μmol) (SM not soluble in MeOH) acetyl chloride (155 mg, 141 μL, 2 Eq, 1.98 mmol) was added dropwise and stirring continued o.n. tlc showed no more sm. The solvent was evaporated under vacuum and the crude was extracted with EA and Sodium bicarbonate saturated solution. The organic phase was dried over sodium sulfate and concentrated under vacuum to give 8-(benzyloxy)-2,6,6,9-tetramethyl-6H-benzo[c]chromen-3-ol (317 mg, 879 μmol, 88.8%) as a brownish solid used to the next step without further purification ¹H NMR (400 MHz, DMSO) δ 9.36 (d, J=1.5 Hz, 1H), 7.49 (d, J=7.7 Hz, 3H), 7.44-7.38 (m, 3H), 7.32 (td, J=6.9, 1.5 Hz, 1H), 6.91 (s, 1H), 6.30 (d, J=1.7 Hz, 1H), 5.15 (s, 2H), 2.21 (s, 3H), 2.10 (s, 3H), 1.49 (s, 6H).

### Step 2: synthesis of 8-(benzyloxy)-2,6,6,9-tetramethyl-6H-benzo[c]chromen-3-yl trifluoromethanesulfonate

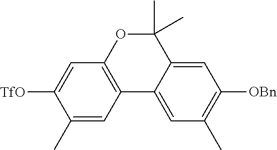 [SMILES: Cc1cc2c(cc1OCc1ccccc1)C(C)(C)Oc1cc(OS(=O)(=O)C(F)(F)F)c(C)cc1-2]

triflicanhydride (352 mg, 211 μL, 1.5 Eq, 1.25 mmol) was added dropwise to a solution of 8-(benzyloxy)-2,6,6,9-tetramethyl-6H-benzo[c]chromen-3-ol (300 mg, 1 Eq, 832 μmol) and pyridine (658 mg, 673 μL, 10 Eq, 8.32 mmol) at 0° C. in DCM 10 mL and the mixture was stirred for 3 h at room temperature. DCM was evaporated under vacuum and the crude was extracted with NH4Cl saturated solution and EA. The organic phase was washed with water once, dried over sodium sulfate and concentrated under vacuum. The crude was purified By FC eluent EA/cyclohexane 0% to 15% to give 8-methoxy-6-oxo-6H-benzo[c]chromen-3-yl trifluoromethanesulfonate (550 mg, 1.47 mmol, 42%). ¹H NMR (400 MHz, CDCl₃) δ 7.53 (s, 1H), 7.49-7.31 (m, 6H), 6.83 (s, 1H), 6.71 (s, 1H), 5.12 (s, 2H), 2.36 (d, J=0.6 Hz, 3H), 2.33 (d, J=0.8 Hz, 3H), 1.58 (s, 6H).

### Step 3: synthesis of tert-butyl (8-(benzyloxy)-2,6,6,9-tetramethyl-6H-benzo[c]chromen-3-yl)carbamate

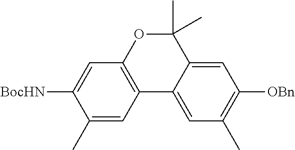 [SMILES: Cc1cc2c(cc1NC(=O)OC(C)(C)C)OC(C)(C)c1cc(OCc3ccccc3)c(C)cc1-2]

t-Bu XPhos (36.2 mg, 0.14 Eq, 85.3 μmol) was added to a suspension of Tris(dibezylideneacetone)dipalladium (39.0 mg, 0.07 Eq, 42.6 μmol) in Toluene 6.5 mL and the mixture was stirred 5 minute at rt. 8-(benzyloxy)-2,6,6,9-tetramethyl-6H-benzo[c]chromen-3-yl trifluoromethanesulfonate (300 mg, 1 Eq, 609 μmol), Tert-butryl carbamate (428 mg, 6 Eq, 3.65 mmol) and cesium carbonate (427 mg, 1.31 mmol) were added and the reaction mixture was refluxed for 1 hours. Water was added and the mixture extracted with EA 3 times. The combined organic phases were dried over sodium sulfate, filtered and concentrated under vacuum. and the crude was loaded on silica and purified by FC eluent EA/cylohexane 0% to 5% to 10% 20% to give tert-butyl (8-(benzyloxy)-2,6,6,9-tetramethyl-6H-benzo[c]chromen-3-yl) carbamate (60 mg, 0.13 mmol, 21%). R_{f} 0.5 (EtOAc/Cyclohexane 20%). MS (APCI+): m/z=460.

### Step 4: synthesis of tert-butyl 8-(benzyloxy)-2,6,6,9-tetramethyl-6H-benzo[c]chromen-3-amine

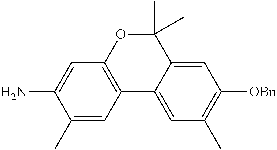 [SMILES: Cc1cc2c(cc1N)OC(C)(C)c1cc(OCc3ccccc3)c(C)cc1-2]

TFA (0.74 g, 0.50 mL, 50 Eq, 6.5 mmol) was added to a solution of tert-butyl (8-(benzyloxy)-2,6,6,9-tetramethyl-6H-benzo[c]chromen-3-yl) carbamate (60 mg, 1 Eq, 0.13 mmol) in DCM (11 mg, 1.3 mL, 0.1 molar, 1 Eq, 0.13 mmol) at 0° C. and stirred at rt for 1 h. Solvent was evaporated under vacuum and the crude was partitioned between EtOAc and sodium bicarbonate saturated solution. The aqueous phase was extracted 3 times with EtOAc. The combined organic phases were dried over sodium sulfate and concentrated under vacuum to give 8-(benzyloxy)-2,6,6,9-tetramethyl-6H-benzo[c]chromen-3-amine (50 mg, 0.14 mmol, 110%) as brownish oil which was used as a crude for next step. MS (APCI+): m/z=360.

### Step 5: synthesis of 3-amino-2,6,6,9-tetramethyl-6H-benzo[c]chromen-8-ol (1)

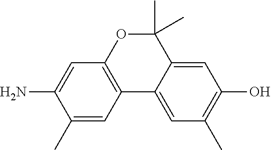 [SMILES: Cc1cc2c(cc1N)OC(C)(C)c1cc(O)c(C)cc1-2]

8-(benzyloxy)-2,6,6,9-tetramethyl-6H-benzo[c]chromen-3-amine (50 mg, 1 Eq, 0.14 mmol) was dissolved in methanol (4.5 mg, 4.6 mL, 0.03 molar, 1 Eq, 0.14 mmol) and DCM (12 mg, 1.4 mL, 0.1 molar, 1 Eq, 0.14 mmol). palladium hydroxide on carbon (9.8 mg, 20% Wt, 0.1 Eq, 14 μmol) was added and the mixture was hydrogenated under atmospheric pressure for 2 h. The crude was filtered over a pad of celite and the crude was purified by FC eluent EtOAc/cyH 0% to 80% to give 3-amino-2,6,6,9-tetramethyl-6H-benzo[c]chromen-8-ol (20 mg, 74 μmol, 53%). R_{f} 0.4 (EtOAc/Cyclohexane 20%).

¹H NMR (400 MHz, DMSO) δ 9.14 (s, 1H), 7.30 (s, 1H), 7.24 (s, 1H), 6.62 (s, 1H), 6.12 (s, 1H), 4.85 (s, 2H), 2.13 (d, J=0.7 Hz, 3H), 2.04-1.87 (m, 3H), 1.44 (s, 6H). MS: m/z: 270 [M+H]⁺.

### 5. Synthesis of 2,6,6,9-tetramethyl-3-(methylamino)-6H-benzo[c]chromen-8-ol (8)

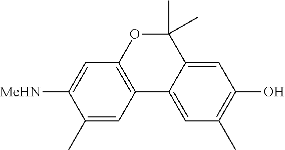 [SMILES: CNc1cc2c(cc1C)-c1cc(C)c(O)cc1C(C)(C)O2]

8 is prepared in 4 steps from tert-butyl (8-(benzyloxy)-2,6,6,9-tetramethyl-6H-benzo[c]chromen-3-yl) carbamate

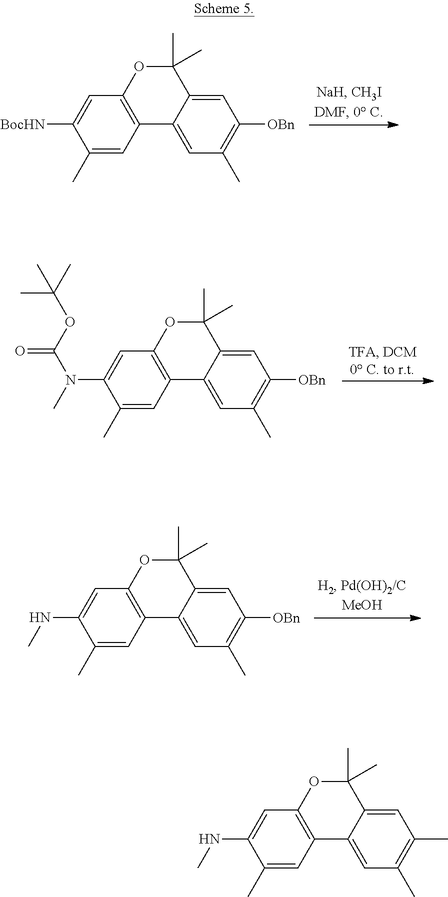

Step 1: synthesis of tert-butyl (8-(benzyloxy)-2,6,6,9-tetramethyl-6H-benzo[c]chromen-3-yl)(methyl)carbamate

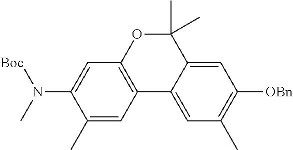 [SMILES: Cc1cc2c(cc1OCc1ccccc1)C(C)(C)Oc1cc(N(C)C(=O)OC(C)(C)C)c(C)cc1-2]

sodium hydride (17 mg, 60% Wt, 2 Eq, 435 μmol) was added to a solution of tert-butyl (8-(benzyloxy)-2,6,6,9-tetramethyl-6H-benzo[c]chromen-3-yl)carbamate (100 mg, 1 Eq, 218 μmol) in DMF (4 mL) at 0° C. then methyl iodide (92.7 mg, 40.8 μL, 3 Eq, 653 μmol) was added and stirring continued for 1 h. NH4Cl Saturate solution and the aqueous phase was extracted with EtOAc. The organic phase was washed with water, dried over sodium sulfate. The solvent was concentrated under vacuum to give m(crude)=100 mg used to the next step without further purification.

¹H NMR (400 MHz, CDCl₃) δ 7.74-7.43 (m, 3H), 7.44-7.35 (m, 2H), 7.33 (d, J=7.3 Hz, 1H), 6.77-6.68 (m, 1H), 6.21 (s, 1H), 5.09 (s, 1H), 2.96 (d, J=0.5 Hz, 3H), 2.89 (d, J=0.7 Hz, 3H), 2.31 (s, 3H), 1.59 (s, 9H).

### Step 2: synthesis 8-(benzyloxy)-N,2,6,6,9-pentamethyl-6H-benzo[c]chromen-3-amine

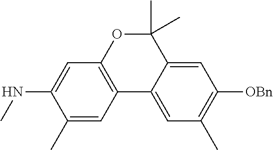 [SMILES: CNc1cc2c(cc1C)-c1cc(C)c(OCc3ccccc3)cc1C(C)(C)O2]

of tert-butyl (8-(benzyloxy)-2,6,6,9-tetramethyl-6H-benzo[c]chromen-3-yl)(methyl)carbamate 100 mg (crude) was dissolved in DCM (3 mL) and treated with TFA (496 mg, 335 μL, 20 Eq, 4.35 mmol) at 0° C. for 1 h then allowed to warm to room temperature overnight. NaHCO₃ saturated solution was added and the aqueous phase was extracted with EtOAc twice. The combined organic phases were dried over sodium sulfate and concentrated under vacuum. The crude material was purified by flash column chromatography on silica (0-50% EtOAc in Hex) to give 8-(benzyloxy)-N,2,6,6,9-pentamethyl-6H-benzo[c]chromen-3-amine (70 mg, 0.19 mmol, 86%). R_{f}=0.5 (EtOAc/Cyclohexane 20%). m/z=373.20 Found 374.2.

### Step 3: synthesis 2,6,6,9-tetramethyl-3-(methylamino)-6H-benzo[c]chromen-8-ol (8)

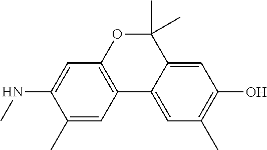 [SMILES: CNc1cc2c(cc1C)-c1cc(C)c(O)cc1C(C)(C)O2]

8-(benzyloxy)-N,2,6,6,9-pentamethyl-6H-benzo[c]chromen-3-amine (70 mg, 1 Eq, 0.19 mmol) was hydrogenated under atmospheric pressure in presence of palladium hydroxide on carbon (13 mg, 20% Wt, 0.1 Eq, 19 μmol) in MeOH (6.0 mg, 3.7 mL, 0.05 molar, 1 Eq, 0.19 mmol) o.n. The crude material was purified by flash column chromatography on silica (0-50% EtOAc in Hex) to give 2,6,6,9-tetramethyl-3-(methylamino)-6H-benzo[c]chromen-8-ol (21 mg, 74 μmol, 40%) as a white solid. R_{f}=0.5 (EtOAc/Cyclohexane 50%).

¹H NMR (400 MHz, DMSO) δ 9.14 (s, 1H), 7.31 (s, 1H), 7.28 (s, 1H), 6.63 (s, 1H), 5.96 (s, 1H), 5.08 (q, J=4.8 Hz, 1H), 2.69 (d, J=4.9 Hz, 3H), 2.13 (d, J=0.7 Hz, 3H), 2.05 (d, J=0.7 Hz, 3H), 1.46 (s, 6H). MS: m/z: 284 [M+H]+.

### 6. Synthesis of 3-methoxy-2,6,6,9-tetramethyl-6H-benzo[c]chromen-8-ol (6)

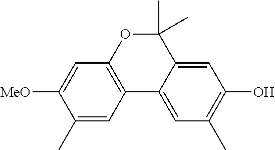 [SMILES: COc1cc2c(cc1C)-c1cc(C)c(O)cc1C(C)(C)O2]

6 was prepared in 4 steps from 8-(benzyloxy)-3-hydroxy-2,9-dimethyl-6H-benzo[c]chromen-6-one

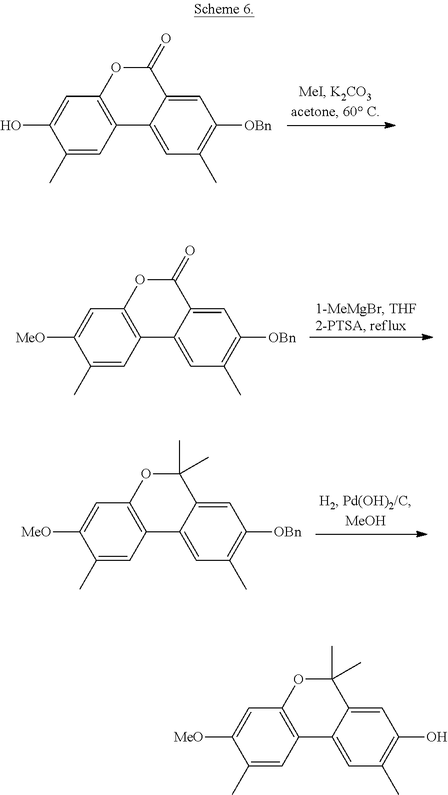 [SMILES: Cc1cc2c(cc1O)oc(=O)c1cc(OCc3ccccc3)c(C)cc12 ; COc1cc2oc(=O)c3cc(OCc4ccccc4)c(C)cc3c2cc1C ; COc1cc2c(cc1C)-c1cc(C)c(OCc3ccccc3)cc1C(C)(C)O2 ; COc1cc2c(cc1C)-c1cc(C)c(O)cc1C(C)(C)O2]

### Step 1: synthesis of 8-(benzyloxy)-3-methoxy-2,9-dimethyl-6H-benzo[c]chromen-6-one

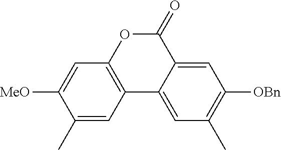 [SMILES: COc1cc2oc(=O)c3cc(OCc4ccccc4)c(C)cc3c2cc1C]

8-(benzyloxy)-3-hydroxy-2,9-dimethyl-6H-benzo[c]chromen-6-one (700 mg, 1 Eq, 2.02 mmol) was dissolved in acetone (117 mg, 40.4 mL, 0.05 molar, 1 Eq, 2.02 mmol) at room temperature. potassium carbonate (1.12 g, 4 Eq, 8.08 mmol) was added followed by methyl iodide (1.43 g, 632 μL, 5 Eq, 10.1 mmol) and the mixture was refluxed overnight. Water was added and the aqueous phase extracted with EOAc 3 times. dried over sodium sulfate and concentrated under vacuum to give a yellowish solid which was triturated in EtOAc to give 8-(benzyloxy)-3-methoxy-2,9-dimethyl-6H-benzo[c]chromen-6-one (680 mg, 1.89 mmol, 93.4%) as a beige solid. ¹H NMR (400 MHz, CDCl₃) δ 7.82-7.79 (m, 1H), 7.78 (s, 1H), 7.71 (d, J=1.0 Hz, 1H), 7.55-7.46 (m, 2H), 7.45-7.38 (m, 2H), 7.37-7.31 (m, 1H), 6.80 (s, 1H), 5.20 (s, 2H), 3.88 (s, 3H), 2.47 (d, J=0.8 Hz, 3H), 2.30 (d, J=0.8 Hz, 3H).

### Step 2: synthesis of 8-(benzyloxy)-3-methoxy-2,6,6,9-tetramethyl-6H-benzo[c]chromene

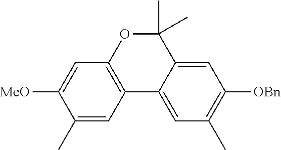 [SMILES: COc1cc2c(cc1C)-c1cc(C)c(OCc3ccccc3)cc1C(C)(C)O2]

8-(benzyloxy)-3-methoxy-2,9-dimethyl-6H-benzo[c]chromen-6-one (350 mg, 1 Eq, 971 Pmol) was dissolved in THF (70.0 mg, 13.9 mL, 0.07 molar, 1 Eq, 971 Pmol) and methylmagnesium bromide (579 mg, 1.62 mL, 3 molar, 5 Eq, 4.86 mmol) was added dropwise at rt. stirring continued o.n. The rm was poured into HCl 1M and extracted twice with EtOAc, dried over sodium sulfate and concentrated under vacuum to give open intermediate 170 mg. Open intermediate was heated in toluene in presence of PTSOH (18.5 mg, 0.1 Eq, 97.1 μmol) for 1 h. The reaction mixture was extracted with EtOAc and Sodium bicarbonate saturated solution. The combined organic phases were dried over sodium sulfate and concentrated under vacuum. The crude was loaded on silica and purified by FC eluent MeOH/DCM 0% to 5% to 10% 20% to give 8-amino-2,6,6,9-tetramethyl-6H-benzo[c]chromen-3-ol (10 mg, 37 μmol, 90%) as a whit solid. ¹H NMR (400 MHz, CDCl₃) δ 7.47-7.43 (m, 3H), 7.43-7.37 (m, 3H), 7.35-7.32 (m, 1H), 6.71 (s, 1H), 6.45 (s, 1H), 5.10 (s, 2H), 3.82 (s, 3H), 2.32 (d, J=0.7 Hz, 3H), 2.21 (d, J=1.0 Hz, 3H), 1.59 (s, 6H).

### Step 3: synthesis of 3-methoxy-2,6,6,9-tetramethyl-6H-benzo[c]chromen-8-ol (6)

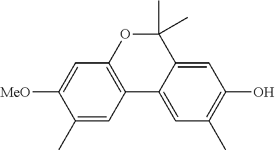 [SMILES: COc1cc2c(cc1C)-c1cc(C)c(O)cc1C(C)(C)O2]

A suspension of 8-(benzyloxy)-3-methoxy-2,6,6,9-tetramethyl-6H-benzo[c]chromene (240 mg, 1 Eq, 641 μmol) and palladium hydroxide on carbon (18.0 mg, 0.2 Eq, 128 μmol) in methanol (20.5 mg, 6.41 mL, 0.1 molar, 1 Eq, 641 μmol) was hydrogenated under atmospheric pressure for 2 hours. The reaction mixture was filtered over a pad of celite. The open intermediate was heated in toluene in presence of p-Toluenesulfonicacidmonohydrate (18.3 mg, 14.7 μL, 0.15 Eq, 96.1 μmol) for 30 min. sodium bicarbonate saturated solution aqueous phase was extracted with EtOAc twice.

The combined organic phases were dried over sodium sulfate and concentrated under vacuum. The crude was loaded on silica and purified by FC eluent EtOAc/Cyclohexane 0% to 20% to give 3-methoxy-2,6,6,9-tetramethyl-6H-benzo[c]chromen-8-ol (170 mg, 598 μmol, 93.3%). R_{f} 0.7 (EtOAc/Cyclohexane 20%). ¹H NMR (400 MHz, DMSO) δ 9.32 (s, 1H), 7.73-7.45 (m, 1H), 7.43 (s, 1H), 6.67 (s, 1H), 6.46 (s, 1H), 3.75 (s, 3H), 2.15 (s, 3H), 2.12 (s, 3H), 1.48 (s, 6H).

### 7. Synthesis of 3-methoxy-2,6,6,9-tetramethyl-6H-benzo[c]chromen-8-amine (4)

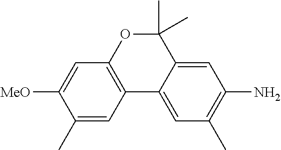 [SMILES: COc1cc2c(cc1C)-c1cc(C)c(N)cc1C(C)(C)O2]

4 was prepared in 3 steps from 3-methoxy-2,6,6,9-tetramethyl-6H-benzo[c]chromen-8-ol (6)

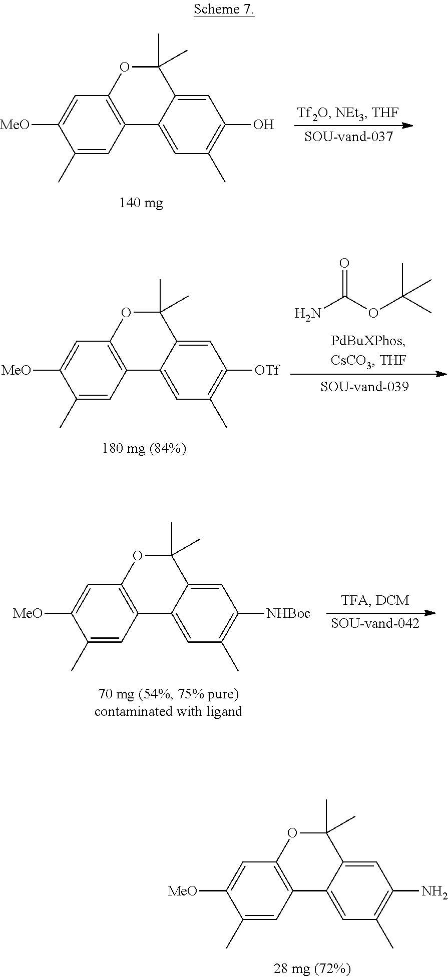

### Step 1: Synthesis of 3-methoxy-2,6,6,9-tetramethyl-6H-benzo[c]chromen-8-yl trifluoromethanesulfonate

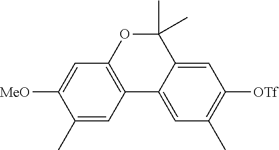 [SMILES: COc1cc2c(cc1C)-c1cc(C)c(OS(=O)(=O)C(F)(F)F)cc1C(C)(C)O2]

Triflic anhydride (360 mg, 215 μL, 2.5 Eq, 1.27 mmol) was added dropwise to a solution of 3-methoxy-2,6,6,9-tetramethyl-6H-benzo[c]chromen-8-ol (145 mg, 1 Eq, 510 μmol) and pyridine (403 mg, 412 μL, 10 Eq, 5.10 mmol) at 0° C. in DCM (10 mL) and the mixture was stirred for 3 h at room temperature. Dichloromethane was evaporated under vacuum and the crude was extracted with NH4Cl saturated solution and EA. The organic phase was washed with water once, dried over sodium sulfate and concentrated under vacuum. The crude was purified By FC eluent EA/cyclohexane 0% to 15% to give 8-methoxy-6-oxo-6H-benzo[c]chromen-3-yl trifluoromethanesulfonate (550 mg, 1.47 mmol, 42%) as a yellowish foam. R_{f} 0.6 (EtOAc/Cyclohexane 40%) used as a crude to the next step without further purification.

### Step 2: synthesis of tert-butyl (3-methoxy-2,6,6,9-tetramethyl-6H-benzo[c]chromen-8-yl) carbamate

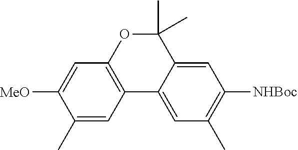 [SMILES: COc1cc2c(cc1C)-c1cc(C)c(NC(=O)OC(C)(C)C)cc1C(C)(C)O2]

t-Bu XPhos (22.8 mg, 0.16 Eq, 53.8 μmol) was added to a suspension of Tris(dibezylideneacetone)dipalladium (24.6 mg, 0.08 Eq, 26.9 μmol) in Toluene (6.5 mL) and the mixture was stirred 5 minutes at rt. 3-methoxy-2,6,6,9-tetramethyl-6H-benzo[c]chromen-8-yl trifluoromethanesulfonate (140 mg, 1 Eq, 336 μmol), Tert-butryl carbamate (197 mg, 5 Eq, 1.68 mmol) and cesium carbonate (427 mg, 1.31 mmol) were added and the reaction mixture was refluxed for 1 hours. Water was added and the mixture extracted with EA 3 times. The combined organic phases were dried over sodium sulfate, filtered and concentrated under vacuum. and the crude was loaded on silica and purified by FC eluent EA/cylohexane 0% to 5% to 10% 20% to give tert-butyl (3-methoxy-2,6,6,9-tetramethyl-6H-benzo[c]chromen-8-yl)carbamate (70 mg, 0.18 mmol, 54%) contaminated with tBuXPhos. MS (APCI+): m/z=384. R_{f}=0.5 (EtOAc/Cyclohexane 20%) as a brownish solid.

### Step 3: synthesis of 3-methoxy-2,6,6,9-tetramethyl-6H-benzo[c]chromen-8-amine (4)

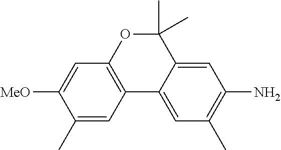 [SMILES: COc1cc2c(cc1C)-c1cc(C)c(N)cc1C(C)(C)O2]

TFA (0.31 g, 0.21 mL, 20 Eq, 2.7 mmol) was added to a mixture of tert-butyl (3-methoxy-2,6,6,9-tetramethyl-6H-benzo[c]chromen-8-yl)carbamate (70 mg, 75% Wt, 1 Eq, 0.14 mmol) in DCM (12 mg, 2.7 mL, 0.05 molar, 1 Eq, 0.14 mmol) at 0° C. and stirring continued at r.t for 3 h. DCM was evaporated under reduced pressure and the crude was extracted with EtOAc/NaHCO₃. The organic phase. was dried over sodium sulfate. The crude was purified biotage eluent EtOAc/cyclohexane 0% to 30% to give 3-methoxy-2,6,6,9-tetramethyl-6H-benzo[c]chromen-8-amine (28 mg, 99 μmol, 72%) as a white solid. R_{f}=0.2 (EtOAc/Cyclohexane 20%). ¹H NMR (400 MHz, DMSO) δ 7.41 (s, 1H), 7.31 (s, 1H), 6.56 (s, 1H), 6.43 (s, 1H), 5.30 (s, 2H), 3.73 (s, 3H), 2.10 (s, 3H), 2.09 (s, 3H), 1.46 (s, 6H). MS: m/z: 270 [M+H]⁺.

### 8. Synthesis of 8-methoxy-2,6,6,9-tetramethyl-6H-benzo[c]chromen-3-ol (5)

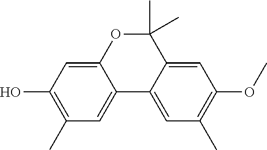 [SMILES: COc1cc2c(cc1C)-c1cc(C)c(O)cc1OC2(C)C]

5 was prepared from methyl resorcinol in 4 steps.

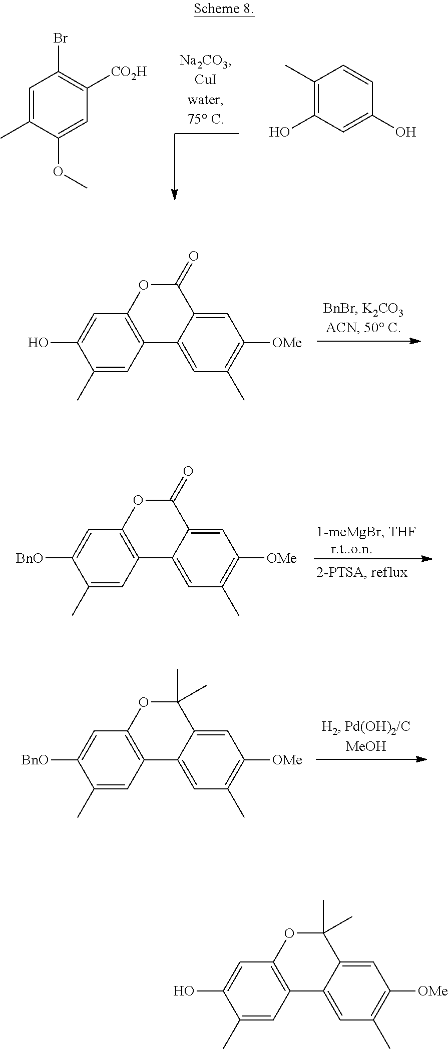

### Step 1: synthesis of 3-hydroxy-8-methoxy-2,9-dimethyl-6H-benzo[c]chromen-6-one

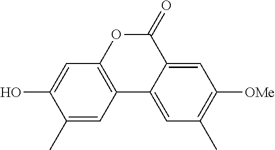 [SMILES: COc1cc2c(=O)oc3cc(O)c(C)cc3c2cc1C]

4-methylbenzene-1,3-diol (2.026 g, 2.0 Eq, 16.32 mmol) was dissolved in water (147.1 mg, 40.80 mL, 0.2 molar, 1 Eq, 8.161 mmol) and sodium carbonate (2.595 g, 3.0 Eq, 24.48 mmol) was added and the mixture heated to 60° C. until everything had dissolved. Then 2-bromo-5-methoxy-4-methylbenzoic acid (2000 mg, 1 Eq, 8.161 mmol) was added and stirring at 75° C. was continued for 4 h. copper(I) iodide (777.1 mg, 0.5 Eq, 4.080 mmol) was added in one portion and the reaction was stirred for 12 h. at 75° C. the precipitate was filtered off and the cake washed successively with water and with HCl 1M then dried under vacuum overnight to give 3-hydroxy-8-methoxy-2,9-dimethyl-6H-benzo[c]chromen-6-one (1.6 g, 5.9 mmol, 73%) as a beige solid. ¹H NMR (400 MHz, DMSO) δ 10.12 (s, 1H), 8.07 (s, 1H), 7.94 (s, 1H), 7.51 (s, 1H), 6.73 (s, 1H), 3.90 (s, 3H), 2.33 (s, 3H), 2.21 (s, 3H).

### Step 2: synthesis of 3-(benzyloxy)-8-methoxy-2,9-dimethyl-6H-benzo[c]chromen-6-one

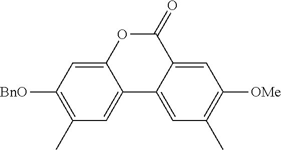 [SMILES: COc1cc2c(=O)oc3cc(OCc4ccccc4)c(C)cc3c2cc1C]

3-hydroxy-8-methoxy-2,9-dimethyl-6H-benzo[c]chromen-6-one (1500 mg, 1 Eq, 5.550 mmol) was dissolved in acetone (322.3 mg, 55.50 mL, 0.1 molar, 1 Eq, 5.550 mmol). benzyl bromide (854.3 mg, 594.1 μL, 0.9 Eq, 4.995 mmol) was added and the mixture was heated at 70° C. overnight. The suspension was filtered off and the filtrate was evaporated under vacuum and the precipitate was triturated in Et2O and filtered to give 3-(benzyloxy)-8-methoxy-2,9-dimethyl-6H-benzo[c]chromen-6-one (1.72 g, 4.77 mmol, 86.0%) as a brownish solid. ¹H NMR (400 MHz, DMSO) δ 8.17 (s, 1H), 8.08 (s, 1H), 7.56 (s, 1H), 7.52-7.47 (m, 2H), 7.42 (t, J=7.4 Hz, 2H), 7.40-7.31 (m, 1H), 7.08 (s, 1H), 5.23 (s, 2H), 3.92 (s, 3H), 2.36 (s, 3H), 2.30 (s, 3H).

### Step 3: Synthesis of 3-(benzyloxy)-8-methoxy-2,6,6,9-tetramethyl-6H-benzo[c]chromene

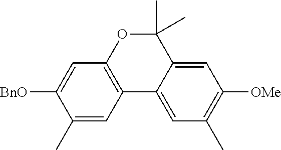 [SMILES: COc1cc2c(cc1C)-c1cc(C)c(OCc3ccccc3)cc1OC2(C)C]

3-(benzyloxy)-8-methoxy-2,9-dimethyl-6H-benzo[c]chromen-6-one (200 mg, 1 Eq, 555 μmol) was dissolved in THF (40.0 mg, 7.93 mL, 0.07 molar, 1 Eq, 555 μmol) and methylmagnesium bromide (304 mg, 851 μL, 3 molar, 4.6 Eq, 2.55 mmol) was added dropwise at rt. stirring continued o.n. The rm was poured into HCl 1M and extracted twice with EtOAc, dried over sodium sulfate and concentrated under vacuum to give open intermediate 170 mg. Open intermediate was heated at 70° C. in toluene in presence of PTSOH (10.6 mg, 0.1 Eq, 55.5 μmol) for 1 h. Toluene was evaporated and the crude was extracted with NaHCO₃ saturated solution, dried over sodium sulfate to give 3-(benzyloxy)-8-methoxy-2,6,6,9-tetramethyl-6H-benzo[c]chromene (160 mg, 427 μmol, 77.0%) as a brownish oil. MS (APCI+): m/z=375. ¹H NMR (400 MHz, CDCl₃) δ 7.55-7.29 (m, 7H), 6.65 (s, 1H), 6.53 (s, 1H), 5.06 (s, 2H), 3.86 (s, 3H), 2.28 (d, J=0.7 Hz, 3H), 2.26 (d, J=0.7 Hz, 3H), 1.62 (s, 6H).

### Step 4: synthesis of 8-methoxy-2,6,6,9-tetramethyl-6H-benzo[c]chromen-3-ol (5)

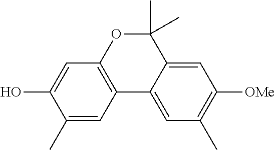 [SMILES: COc1cc2c(cc1C)-c1cc(C)c(O)cc1OC2(C)C]

A suspension of 3-(benzyloxy)-8-methoxy-2,6,6,9-tetramethyl-6H-benzo[c]chromene (800 mg, 1 Eq, 2.14 mmol) and palladium hydroxide on carbon (300 mg, 20% Wt, 0.2 Eq, 427 μmol) in MeOH (5 mL) was hydrogenated under atmospheric pressure over 2 h. The suspension was filtered over a pad of celite and the solvent was evaporated under vacuum to give 8-methoxy-2,6,6,9-tetramethyl-6H-benzo[c]chromen-3-ol (600 mg, 2.11 mmol, 98.8%) as a white foam. R_{f}0.5 EA/Hexane 10/90. ¹H NMR (400 MHz, CDCl₃) δ 14.11 (s, 1H), 12.22 (d, J=0.9 Hz, 1H), 12.18 (s, 1H), 11.55 (s, 1H), 11.07 (s, 1H), 8.57 (s, 3H), 8.08 (s, 2H), 6.92 (d, J=0.7 Hz, 3H), 6.86 (d, J=0.7 Hz, 3H), 6.28 (s, 6H).

### 9. Synthesis of 8-methoxy-2,6,6,9-tetramethyl-6H-benzo[c]chromen-3-amine (3)

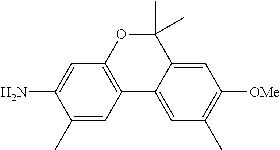 [SMILES: COc1cc2c(cc1C)-c1cc(C)c(N)cc1OC2(C)C]

3 was prepared from 6 in 3 steps.

### Step 1: synthesis of 8-methoxy-2,6,6,9-tetramethyl-6H-benzo[c]chromen-3-yl trifluoromethanesulfonate

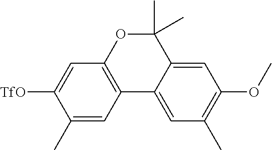 [SMILES: COc1cc2c(cc1C)-c1cc(C)c(OS(=O)(=O)C(F)(F)F)cc1OC2(C)C]

Triflicanhydride (570 mg, 342 μL, 2.5 Eq, 2.02 mmol) was added dropwise to a solution of 8-methoxy-2,6,6,9-tetramethyl-6H-benzo[c]chromen-3-ol (230 mg, 1 Eq, 809 μmol) and pyridine (640 mg, 654 μL, 10 Eq, 8.09 mmol) at 0° C. in DCM (10 mL) and the mixture was stirred for 3 h at room temperature. DCM was evaporated under vacuum and the crude was extracted with NH4Cl saturated solution and EA. The organic phase was washed with water once, dried over sodium sulfate and concentrated under vacuum. The crude was purified By FC eluent EA/cyclohexane 0% to 15% to give 8-methoxy-6-oxo-6H-benzo[c]chromen-3-yl trifluoromethanesulfonate (550 mg, 1.47 mmol, 42%) as a white solid. R_{f} 0.6 (EA/cyclohexane40%) used as a crude.

### Step 2: synthesis of tert-butyl (8-methoxy-2,6,6,9-tetramethyl-6H-benzo[c]chromen-3-yl) carbamate

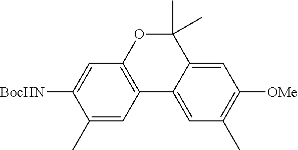 [SMILES: COc1cc2c(cc1C)-c1cc(C)c(NC(=O)OC(C)(C)C)cc1OC2(C)C]

t-BuXPhos (42.8 mg, 0.14 Eq, 101 μmol) was added to a suspension of Tris(dibenzylideneacetone)dipalladium (46.2 mg, 0.07 Eq, 50.4 μmol) in Toluene (6.5 mL) and the mixture was stirred 5 minute at rt. 8-methoxy-2,6,6,9-tetramethyl-6H-benzo[c]chromen-3-yl trifluoromethanesulfonate (300 mg, 1 Eq, 720 μmol), Tert-butryl carbamate (338 mg, 4 Eq, 2.88 mmol) and cesium carbonate (427 mg, 1.31 mmol) were added and the reaction mixture was refluxed overnight. NH₄Cl saturated solution was added and the mixture extracted with EA 3 times. The combined organic phases were dried over sodium sulfate, filtered and concentrated under vacuum and the crude was loaded on silica and purified by FC eluent EA/cylohexane 0% to 5% to 10% 20% to give tert-butyl (8-methoxy-2,6,6,9-tetramethyl-6H-benzo[c]chromen-3-yl) carbamate (145 mg, 378 μmol, 52.5%) contaminated with tBuXPhos. The product was used to the next step without further purification. R_{f} 0.5 (EA/cyclohexane 20%).

### Step 3: synthesis of 8-methoxy-2,6,6,9-tetramethyl-6H-benzo[c]chromen-3-amine 3

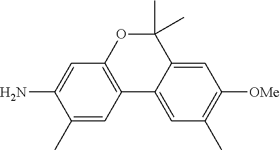 [SMILES: COc1cc2c(cc1C)-c1cc(C)c(N)cc1OC2(C)C]

tert-butyl (8-methoxy-2,6,6,9-tetramethyl-6H-benzo[c]chromen-3-yl)carbamate (70 mg, 1 Eq, 0.18 mmol) was dissolved in dry DCM (16 mg, 12 μL, 1 Eq, 0.18 mmol) and cooled to 0° C. then TFA (0.21 g, 0.14 mL, 10 Eq, 1.8 mmol) was added dropwise and stirring continued at rt for 2 h. Na2CO3 sat. sol. was added and the mixture extracted with EA twice. The combined organic phases were dried over sodium sulfate, filtered and concentrated under vacuum. The crude was loaded on silica and purified by FC eluent EA/cylohexane 0% to 5% to 10% 20% to give 8-methoxy-2,6,6,9-tetramethyl-6H-benzo[c]chromen-3-amine (20 mg, 71 μmol, 39%). R_{f} 0.2 (EA/cyclohexane 20%). ¹H NMR (400 MHz, DMSO) δ 7.38 (s, 1H), 7.28 (s, 1H), 6.76 (s, 1H), 6.13 (s, 1H), 4.90 (s, 2H), 3.79 (s, 3H), 2.32-2.09 (s, 3H), 2.04 (s, 3H), 1.50 (s, 6H).

### 10. Synthesis of 7-amino-1,6,9,9-tetramethyl-9H-fluorene-2,4-diol (14a)

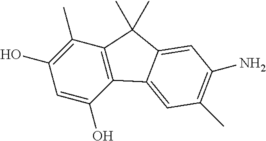 [SMILES: Cc1cc2c(cc1N)C(C)(C)c1c(C)c(O)cc(O)c1-2]

14a was prepared from tert-butyl (3-methoxy-2,6,6,9-tetramethyl-6H-benzo[c]chromen-8-yl) carbamate in one step by treatment with BBr₃ via a rearrangement involving the formation of a carbocation.

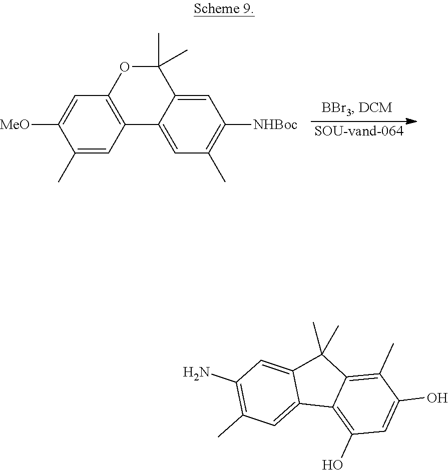 [SMILES: COc1cc2c(cc1C)-c1cc(C)c(NC(=O)OC(C)(C)C)cc1C(C)(C)O2 ; Cc1cc2c(cc1N)C(C)(C)c1c(C)c(O)cc(O)c1-2]

BBr₃ (235.2 mg, 938.7 μL, 1 molar, 4 Eq, 938.7 μmol) was added to a suspension of tert-butyl (3-methoxy-2,6,6,9-tetramethyl-6H-benzo[c]chromen-8-yl)carbamate (90.00 mg, 1 Eq, 234.7 μmol) in DCM (19.93 mg, 4.694 mL, 0.05 molar, 1 Eq, 234.7 μmol) at −78° C. over 5 min and the mixture was allowed to warm to r.t o.n. The reaction mixture was quenched with NaHCO₃ saturated solution and the aqueous phase was extracted with EtOAc. The combined organic phases were dried over sodium sulfate and the organic residue was subjected to column chromatography biotage FC EtOAc/cyclohexane 0% to 20% to give MeOH/DCM 0% to 20% to give 7-amino-1,6,9,9-tetramethyl-9H-fluorene-2,4-diol (20 mg, 74 μmol, 32%) as a white solid. R_{f} 0.5 (MeOH/DCM 10%). ¹H NMR (400 MHz, DMSO) δ 9.13 (s, 1H), 8.89 (s, 1H), 7.40 (s, 1H), 6.62 (s, 1H), 6.32 (s, 1H), 4.78 (s, 2H), 2.16 (s, 3H), 2.06 (s, 3H), 1.39 (d, J=2.1 Hz, 6H).

### 11. Synthesis of 2,6,6,9-tetramethyl-8-(methylamino)-6H-benzo[c]chromen-3-ol (14)

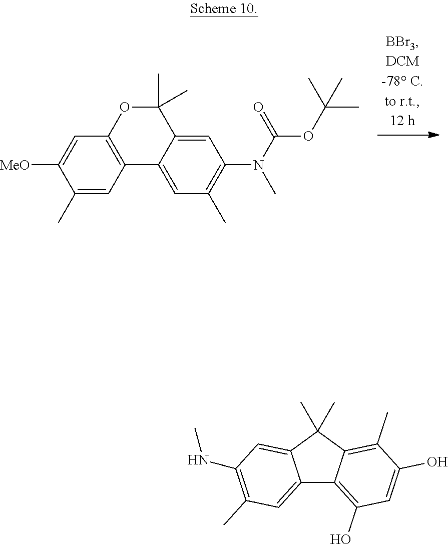

BBr₃ (100.8 mg, 402.5 μL, 1 molar, 4 Eq, 402.5 μmol) was added to a suspension of tert-butyl (3-methoxy-2,6,6,9-tetramethyl-6H-benzo[c]chromen-8-yl)(methyl)carbamate (40.00 mg, 1 Eq, 100.6 μmol) in DCM (8.546 mg, 1.006 mL, 0.1 molar, 1 Eq, 100.6 μmol) at −78° C. over 5 min and the mixture was allowed to warm to r.t o.n. The reaction mixture was quenched with NaHCO₃ saturated solution and the aqueous phase was extracted with EtOAc. The combined organic phases were dried over sodium sulfate and the organic residue was subjected to column chromatography biotage FC EtOAc/cyclohexane 0% to 20% to give MeOH/DCM 0% to 20% to give 2,6,6,9-tetramethyl-8-(methylamino)-6H-benzo[c]chromen-3-ol (19 mg, 67 μmol, 67%) as a brownish solid. R_{f} 0.5 (MeOH/DCM 10%). ¹H NMR (400 MHz, DMSO) δ 9.15 (s, 1H), 8.91 (s, 1H), 7.45 (s, 1H), 6.50 (s, 1H), 6.33 (s, 1H), 4.86 (s, 1H), 2.78 (s, 3H), 2.17 (s, 3H), 2.09 (s, 3H), 1.44 (s, 3H), 1.43 (s, 3H). MS (APCI+): m/z=284.

### 12. Synthesis of 3,8,9,9-tetramethyl-7-(methylamino)-9H-fluorene-2,5-diol (15)

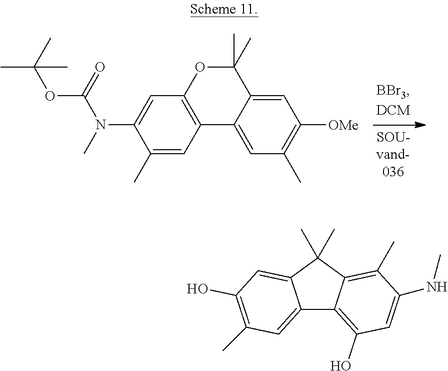

BBr₃ (0.11 g, 0.45 mL, 1 molar, 3 Eq, 0.45 mmol) was added to a suspension of tert-butyl (8-methoxy-2,6,6,9-tetramethyl-6H-benzo[c]chromen-3-yl)(methyl)carbamate (60 mg, 1 Eq, 0.15 mmol) in DCM (13 mg, 3.0 mL, 0.05 molar, 1 Eq, 0.15 mmol) at −78° C. over 2 min and the mixture was allowed to warm to r.t o.n. The reaction mixture was quenched with NaHCO₃ saturated solution, and the aqueous phase was extracted with EtOAc. The combined organic phases were dried over sodium sulfate and the organic residue was subjected to column chromatography biotage FC MeOH/DCM 0% to 20% to give 3,8,9,9-tetramethyl-7-(methylamino)-9H-fluorene-2,5-diol (20 mg, 71 μmol, 47%) as a white solid. R_{f} 0.5 (MeOH/DCM 10%). ¹H NMR (400 MHz, DMSO) δ 9.14 (s, 1H), 8.88 (s, 1H), 7.49 (s, 1H), 6.75 (s, 1H), 6.03 (s, 1H), 4.88 (s, 1H), 2.77-2.70 (m, 3H), 2.16 (s, 3H), 2.14 (s, 3H), 1.44 (s, 3H), 1.44 (s, 3H). MS (APCI+): m/z=284.

### 13. Synthesis of 7-amino-3,8,9,9-tetramethyl-9H-fluorene-2,5-diol (13)

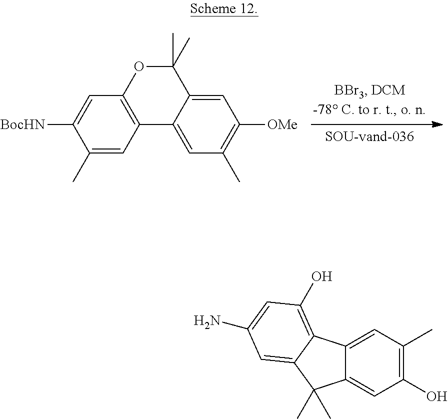 [SMILES: COc1cc2c(cc1C)-c1cc(C)c(NC(=O)OC(C)(C)C)cc1OC2(C)C ; Cc1cc2c(cc1O)C(C)(C)c1cc(N)cc(O)c1-2]

BBr₃ (0.18 g, 0.73 mL, 1 molar, 4 Eq, 0.73 mmol) was added to a suspension of tert-butyl (8-methoxy-2,6,6,9-tetramethyl-6H-benzo[c]chromen-3-yl)carbamate (70 mg, 1 Eq, 0.18 mmol) in DCM (13 mg, 3.0 mL, 0.05 molar, 1 Eq, 0.15 mmol) at −78° C. over 2 min and the mixture was allowed to warm to r.t o.n. The reaction mixture was quenched with NaHCO₃ saturated solution and the aqueous phase was extracted with EtOAc. The combined organic phases were dried over sodium sulfate and the organic residue was subjected to column chromatography biotage FC MeOH/DCM 0% to 20% to give 7-amino-3,8,9,9-tetramethyl-9H-fluorene-2,5-diol (20 mg, 74 μmol, 41%) as a white solid. R_{f}0.5 (MeOH/DCM 10%). ¹H NMR (400 MHz, DMSO) δ 9.00 (s, 1H), 8.82 (s, 1H), 7.44 (s, 1H), 6.70 (s, 1H), 6.13 (s, 1H), 4.66 (s, 2H), 2.11 (d, J=0.7 Hz, 3H), 2.09 (s, 3H), 1.39 (s, 6H). MS (APCI+): m/z=270.

### 14. Synthesis of 2,9-dimethylspiro[benzo[c]chromene-6,1′-cyclopropane]-3,8-diol (7)

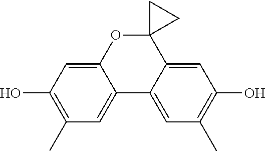 [SMILES: Cc1cc2c(cc1O)OC1(CC1)c1cc(O)c(C)cc1-2]

7 was prepared from intermediate methyl 5-(benzyloxy)-2-bromo-4-methylbenzoate and 2-(4-(benzyloxy)-2-fluoro-5-methylphenyl)-4,4,5,5-tetramethyl-1,3,2-dioxaborolane synthesized according in 4 steps.

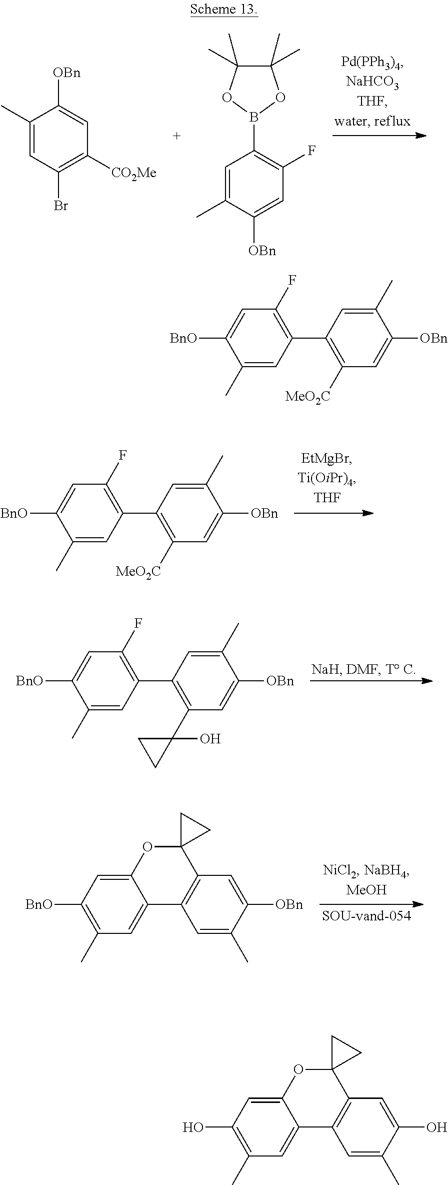

### Step 1: synthesis of methyl 4,4′-bis(benzyloxy)-2′-fluoro-5,5′-dimethyl-[1,1′-biphenyl]-2-carboxylate

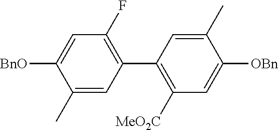 [SMILES: COC(=O)c1cc(OCc2ccccc2)c(C)cc1-c1cc(C)c(OCc2ccccc2)cc1F]

methyl 5-(benzyloxy)-2-bromo-4-methylbenzoate (1000 mg, 1 Eq, 2.983 mmol), 2-(4-(benzyloxy)-2-fluoro-5-methylphenyl)-4,4,5,5-tetramethyl-1,3,2-dioxaborolane (1.225 g, 1.2 Eq, 3.580 mmol) and 1,2-Bis(diphenylphosphino)ethane palladium(II)dichloride (171.8 mg, 0.1 Eq, 298.3 μmol) were suspended in THF (215.1 mg, 29.83 mL, 0.1 molar, 1 Eq, 2.983 mmol) and degassed with Nitrogen for 5 minutes. A solution of sodium bicarbonate (551.4 mg, 2.2 Eq, 6.563 mmol) in water (10 g, 10 mL, 0.3 molar, 1.0 Eq, 3.0 mmol) was added at r.t. and the mixture was refluxed o.n. After cooling to room temperature, NH4Cl saturated solution was added and the aqueous phase was extracted with EA twice. The combined organic phases were dried over sodium sulfate, concentrated under vacuum and the organic residue was subjected to column chromatography biotage FC EA/cyclohexane 0% to 10% to give methyl 4,4′-bis(benzyloxy)-2′-fluoro-5,5′-dimethyl-[1,1′-biphenyl]-2-carboxylate (870 mg, 1.85 mmol, 62.0%) as a white solid. R_{f} 0.3 (EA/cyclohexane 5%). ¹H NMR (400 MHz, CDCl₃) δ 7.60-7.32 (m, 11H), 7.15 (t, J=0.9 Hz, 1H), 7.08 (dq, J=8.6, 0.9 Hz, 1H), 6.68 (dd, J=11.7, 1.2 Hz, 1H), 5.18 (s, 2H), 5.11 (s, 2H), 3.73 (d, J=1.1 Hz, 3H), 2.36 (d, J=0.8 Hz, 3H), 2.30 (d, J=1.2 Hz, 3H).

### Step 2: Synthesis of 1-(4,4′-bis(benzyloxy)-2′-fluoro-5,5′-dimethyl-[1,1′-biphenyl]-2-yl) cyclopropan-1-ol

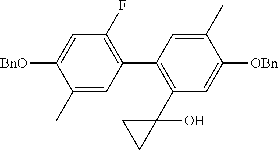 [SMILES: Cc1cc(-c2cc(C)c(OCc3ccccc3)cc2C2(O)CC2)c(F)cc1OCc1ccccc1]

Under nitrogen atmosphere, Ethylmagnesium bromide solution/1M THF (1.70 g, 12.8 mL, 1 molar, 15 Eq, 12.8 mmol) was added dropwise over 5 minutes at 0° C. to a solution of methyl 4,4′-bis(benzyloxy)-2′-fluoro-5,5′-dimethyl-[1,1′-biphenyl]-2-carboxylate (400 mg, 1 Eq, 850 μmol) and Titanium tetraisopropoxide (747 mg, 0.78 mL, 97% Wt, 3 Eq, 2.55 mmol) in THF (61.3 mg, 10.6 mL, 0.08 molar, 1 Eq, 850 μmol). The reaction was then stirred at 0° C. for 30 min: TLC showed no more SM. The mixture was quenched with HCl 1 M. After filtration, the solution was extracted with 3×30 mL of EtOAc, washed with distilled water, and dried over MgSO₄, followed by filtration and concentration. The organic residue was subjected to column chromatography biotage FC EtOAc/cyclohexane 0% to 20% to give 1-(4,4′-bis(benzyloxy)-2′-fluoro-5,5′-dimethyl-[1,1′-biphenyl]-2-yl)cyclopropan-1-ol (170 mg, 363 μmol, 42.7%) R_{f} 0.2 (EA/cyclohexane 10%) as a colorless oil. ¹H NMR (400 MHz, CDCl₃) δ 7.52-7.33 (m, 10H), 7.16 (s, 1H), 7.06 (dd, J=8.9, 0.9 Hz, 1H), 7.02 (d, J=0.9 Hz, 1H), 6.72 (d, J=12.0 Hz, 1H), 5.15 (s, 2H), 5.11 (s, 2H), 2.29 (d, J=0.7 Hz, 3H), 2.26 (d, J=0.9 Hz, 3H), 0.99-0.79 (m, 2H), 0.65-0.55 (m, 2H).

### Step 3: Synthesis of 3,8-bis(benzyloxy)-2,9-dimethylspiro[benzo[c]chromene-6,1′-cyclopropane]

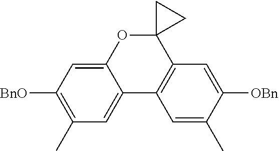 [SMILES: Cc1cc2c(cc1OCc1ccccc1)OC1(CC1)c1cc(OCc3ccccc3)c(C)cc1-2]

A solution of 1-(4,4′-bis(benzyloxy)-2′-fluoro-5,5′-dimethyl-[1,1′-biphenyl]-2-yl)cyclopropan-1-ol (170.00 mg, 1 Eq, 362.81 μmol) in DMF (4 mL) was heated at 110° C. Sodium hydride (61 mg, 1.53 mmol, 5 eq., 60% in oil) was added to was added in one portion and heating continued for 5 min. TLC showed no more SM. The reaction mixture was diluted with EtOAc and poured in a solution of ice and NaHCO₃ saturated solution. The aqueous phase was extracted with EtOAc twice and the combined organic phases were dried over sodium sulfate and evaporated under vacuum. The crude was purified by FC eluent EtOAc/cyclohexane 0% to 20% to give 3,8-bis(benzyloxy)-2,9-dimethylspiro[benzo[c]chromene-6,1′-cyclopropane] (50 mg, 0.11 mmol, 31%) as colourless oil. R_{f} 0.4 (EA/cyclohexane 5%). ¹H NMR (400 MHz, DMSO) δ 7.64-7.57 (m, 1H), 7.57-7.53 (m, 1H), 7.51-7.43 (m, 4H), 7.40 (td, J=7.1, 1.0 Hz, 4H), 7.35-7.30 (m, 2H), 6.64 (s, 1H), 6.54 (s, 1H), 5.13 (s, 2H), 5.09 (s, 2H), 2.23 (d, J=0.7 Hz, 3H), 2.19 (d, J=0.7 Hz, 3H), 1.24-1.16 (m, 2H), 1.11 (t, J=3.4 Hz, 2H).

### Step 4: synthesis 2,9-dimethylspiro[benzo[c]chromene-6,1′-cyclopropane]-3,8-diol (7)

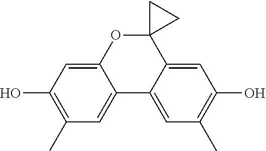 [SMILES: Cc1cc2c(cc1O)OC1(CC1)c1cc(O)c(C)cc1-2]

3,8-bis(benzyloxy)-2,9-dimethylspiro[benzo[c]chromene-6,1′-cyclopropane] (50.00 mg, 1.00 Eq, 111.5 μmol) was dissolved in MeOH (4 mL). Nickelous chloride hexahydrate (264.9 mg, 10 Eq, 1.115 mmol) was added. Sodium tetrahydroborate (84.34 mg, 78.89 μL, 20 Eq, 2.229 mmol) was carefully added portionwise (gas evolution). TLC showed no more starting material. The mixture was filtered off and the crude was loaded on silica gel and purified by FC eluent MeOH/DCM 0% to 4% to give 2,9-dimethylspiro[benzo[c]chromene-6,1′-cyclopropane]-3,8-diol (20 mg, 75 μmol, 67%) as a white solid. R_{f} 0.4 (MeOH/DCM 4%). ¹H NMR (400 MHz, DMSO) δ 9.36 (s, 1H), 9.25 (s, 1H), 7.41 (s, 1H), 7.41 (s, 1H), 6.32 (s, 1H), 6.25 (s, 1H), 2.14 (s, 3H), 2.10 (s, 3H), 1.22-1.10 (m, 2H), 0.98-0.87 (m, 2H). MS: m/z: 269 [M+H]⁺.

### 15. Synthesis of 2,9-dimethylspiro[benzo[c]chromene-6,1′-cyclopentane](9)

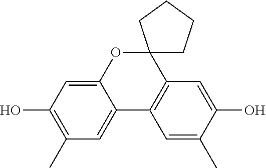 [SMILES: Cc1cc2c(cc1O)OC1(CCCC1)c1cc(O)c(C)cc1-2]

9 was prepared from intermediate methyl 4,4′-bis(benzyloxy)-2′-fluoro-5,5′-dimethyl-[1,1′-biphenyl]-2-carboxylate in 3 steps.

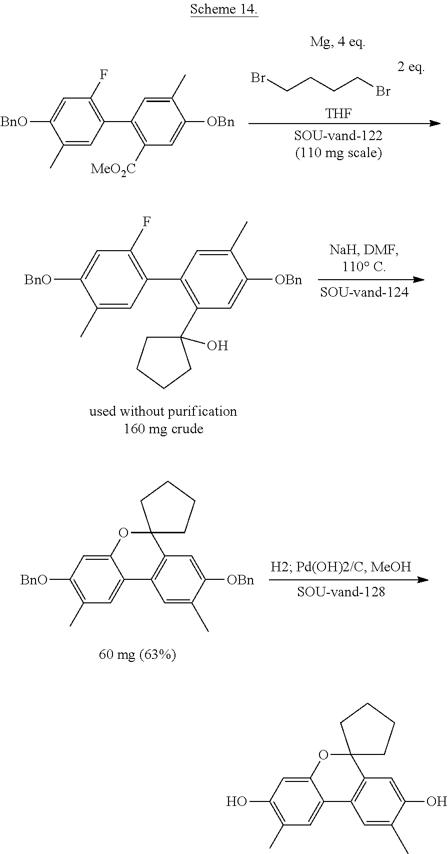 [SMILES: COC(=O)c1cc(OCc2ccccc2)c(C)cc1-c1cc(C)c(OCc2ccccc2)cc1F ; BrCCCCBr ; Cc1cc(-c2cc(C)c(OCc3ccccc3)cc2C2(O)CCCC2)c(F)cc1OCc1ccccc1 ; Cc1cc2c(cc1OCc1ccccc1)OC1(CCCC1)c1cc(OCc3ccccc3)c(C)cc1-2 ; Cc1cc2c(cc1O)OC1(CCCC1)c1cc(O)c(C)cc1-2 ; "THF"]

### Step 1: Synthesis of 1-(4,4′-bis(benzyloxy)-2′-fluoro-5,5′-dimethyl-[1,1′-biphenyl]-2-yl) cyclopentan-1-ol

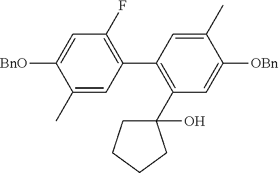 [SMILES: Cc1cc(-c2cc(C)c(OCc3ccccc3)cc2C2(O)CCCC2)c(F)cc1OCc1ccccc1]

To a dry magnesium (39.7 mg, 4.8 Eq, 1.6322 mmol) in dry diethyl ether (25.20 mg, 6.8007 mL, 0.05 molar, 1 Eq, 340.03 μmol) was added 1,4-dibromobutane (440.52 mg, 242.0 μL, 6 Eq, 2.0402 mmol) at rt and the reaction mixture was stirred for 2 h. to this turbid solution was added a solution of methyl 4,4′-bis(benzyloxy)-2′-fluoro-5,5′-dimethyl-[1,1′-biphenyl]-2-carboxylate (160.00 mg, 1 Eq, 340.03 μmol) in dry THF (1 mL) and the reaction stirred for 5 h. tlc showed still some sm (a polar product was formed, TLC MS showed M-18). The reaction was quenched with NH4Cl saturated solution and extracted with EtOAc dried over sodium sulfate and evaporated under vacuum to give methyl 4,4′-bis(benzyloxy)-2′-fluoro-5,5′-dimethyl-[1,1′-biphenyl]-2-carboxylate (160.00 mg, 1 Eq, 340.03 μmol) crude used to the next step without further purification. R_{f}=0.2 (EA/cyclohexane 10%).

### Step 2: Synthesis 3,8-bis(benzyloxy)-2,9-dimethylspiro[benzo[c]chromene-6,1′-cyclopentane]

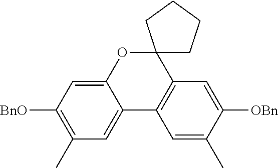 [SMILES: Cc1cc2c(cc1OCc1ccccc1)OC1(CCCC1)c1cc(OCc3ccccc3)c(C)cc1-2]

In a round bottom flask, 1-(4,4′-bis(benzyloxy)-2′-fluoro-5,5′-dimethyl-[1,1′-biphenyl]-2-yl)cyclopentan-1-ol (100 mg, 1 Eq, 201 μmol) was dissolved in dry DMF (3 mL) and heated at 120° C. then NaH (40 mg, 60% Wt, 5 Eq, 1.01 mmol) was added portionwise and stirring continued at 120° C. for 10 min. The mixture was cooled down to room temperature, NaHCO₃ saturated solution was added and the aqueous phase was extracted with EA twice. The combined organic phases were dried over sodium sulfate and concentrated under vacuum. The crude was loaded on silica gel and purified by FC eluent EtOAc/Cyh 0% to 10% to give 3,8-bis(benzyloxy)-2,9-dimethylspiro[benzo[c]chromene-6,1′-cyclopentane] (60 mg, 0.13 mmol, 63%) as a white solid. R_{f} 0.6 (EA/cyclohexane 10%). MS (APCI+): m/z=476. ¹H NMR (400 MHz, CDCl₃) δ 7.51-7.42 (m, 6H), 7.39 (td, J=7.4, 1.7 Hz, 4H), 7.33 (td, J=7.0, 1.8 Hz, 2H), 6.72 (s, 1H), 6.52 (s, 1H), 5.10 (s, 2H), 5.05 (s, 2H), 2.32 (d, J=0.7 Hz, 3H), 2.27 (d, J=0.9 Hz, 3H), 2.25-2.15 (m, 2H), 2.03-1.82 (m, 4H), 1.81-1.70 (m, 2H).

### Step 3: Synthesis of 2,9-dimethylspiro[benzo[c]chromene-6,1′-cyclopentane]-3,8-diol (9)

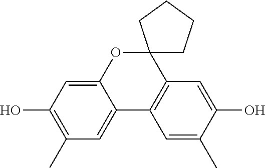 [SMILES: Cc1cc2c(cc1O)OC1(CCCC1)c1cc(O)c(C)cc1-2]

3,8-bis(benzyloxy)-2,9-dimethylspiro[benzo[c]chromene-6,1′-cyclopentane] (60 mg, 1 Eq, 0.13 mmol) was dissolved in methanol (4.0 mg, 2.5 mL, 0.05 molar, 1 Eq, 0.13 mmol) at room temperature. Palladium hydroxide on carbon (18 mg, 20% Wt, 0.2 Eq, 25 μmol) was added and the suspension was hydrogenated under atmospheric pressure over 4 h. The suspension was filtered over a pad of celite and the solvent was concentrated under vacuum. The crude was loaded on silica gel and purified by FC eluent MeOH/DCM 0% to 10% to give 3,8-bis(benzyloxy)-2,9-dimethylspiro[benzo[c]chromene-6,1′-cyclopentane] (60 mg, 0.13 mmol, 63%) as a white solid. R_{f}0.4 (MeOH/DCM 10%). MS: m/z: 297 [M+H]⁺. ¹H NMR (400 MHz, DMSO) δ 9.27 (s, 1H), 9.25 (s, 1H), 7.37 (d, J=2.0 Hz, 2H), 6.66 (s, 1H), 6.28 (s, 1H), 2.14 (d, J=0.7 Hz, 3H), 2.09 (s, 3H), 2.05-2.00 (m, 2H), 1.91-1.67 (m, 6H).

### 16. Synthesis of 9-ethyl-2-methylspiro[benzo[c]chromene-6,1′-cyclobutane]-3,8-diol (10)

11 was prepared from 1-(benzyloxy)-5-bromo-2-ethyl-4-iodobenzene and (4-(benzyloxy)-2-fluoro-5-methylphenyl)boronic acid (see patent lamazentis) in 4 steps.

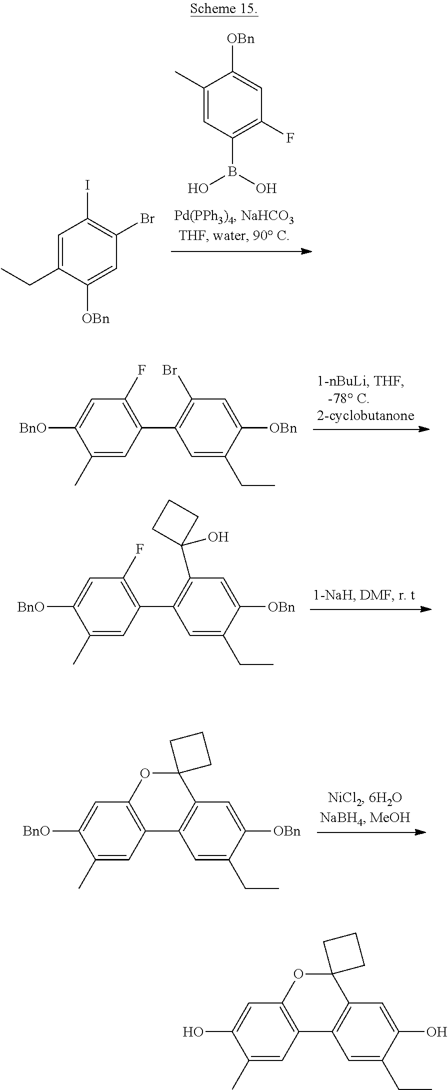 [SMILES: CCc1cc(I)c(Br)cc1OCc1ccccc1 ; Cc1cc(B(O)O)c(F)cc1OCc1ccccc1 ; CCc1cc(-c2cc(C)c(OCc3ccccc3)cc2F)c(Br)cc1OCc1ccccc1 ; CCc1cc(-c2cc(C)c(OCc3ccccc3)cc2F)c(C2(O)CCC2)cc1OCc1ccccc1 ; CCc1cc2c(cc1OCc1ccccc1)C1(CCC1)Oc1cc(OCc3ccccc3)c(C)cc1-2 ; CCc1cc2c(cc1O)C1(CCC1)Oc1cc(O)c(C)cc1-2]

### Step 1: Synthesis of 4,4′-bis(benzyloxy)-2-bromo-5-ethyl-2′-fluoro-5′-methyl-1,1′-biphenyl

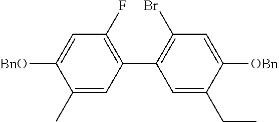 [SMILES: CCc1cc(-c2cc(C)c(OCc3ccccc3)cc2F)c(Br)cc1OCc1ccccc1]

1-(benzyloxy)-5-bromo-4-iodo-2-methylbenzene (370 mg, 0.918 mmol) and (4-(benzyloxy)-2-fluoro-5-methylphenyl)boronic acid (334 mg, 1.29 mmol, 1.4 eq.) were dissolved in dioxane (10 mL). Tetrakis(triphenylphosphine)palladium(o) (85 mg, 0.073 mmol, 0.08 eq.) was added and the solution was degassed for 5 min. then sodium bicarbonate saturated solution (1.8 mL, 1.84 mmol, 2 eq.) was added dropwise and the mixture was heated at 90° C. overnight. Water was added and the mixture was extracted with EtOAc twice. The combined organic phases were dried over sodium sulfate and concentrated under vacuum. The crude was purified by FC biotage EA/CyH 0% to 5% to give 4,4′-bis(benzyloxy)-2-bromo-2′-fluoro-5,5′-dimethyl-1,1′-biphenyl (340 mg, 0.692 mmol, 75%) as colorless oil. R_{f} 0.3 (EA/cyclohexane 10%). ¹H NMR (400 MHz, CDCl₃) δ 7.50-7.31 (m, 10H), 7.19 (s, 1H), 7.12-7.06 (m, 1H), 7.06-7.02 (m, 1H), 6.70 (d, J=11.4 Hz, 1H), 5.09 (s, 4H), 2.95-2.54 (m, 2H), 1.21 (t, J=7.5 Hz, 3H).

### Step 2: Synthesis of 4,4′-bis(benzyloxy)-2-bromo-5,5′-diethyl-2′-fluoro-1,1′-biphenyl

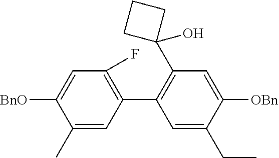 [SMILES: CCc1cc(-c2cc(C)c(OCc3ccccc3)cc2F)c(C2(O)CCC2)cc1OCc1ccccc1]

nBuLi (53.28 mg, 519.78 μL, 1.6 molar, 1.8 Eq, 831.65 μmol) was added to a solution of 4,4′-bis(benzyloxy)-2-bromo-5,5′-diethyl-2′-fluoro-1,1′-biphenyl (240.00 mg, 1 Eq, 462.03 μmol) in THF (7 mL) at −78° C. The mixture was stirred for 30 min at −78° C. then 1-Oxocyclobutane (161.9 mg, 173 μL, 5 Eq, 2.3101 mmol) was added dropwise and the mixture was allowed to warm to rt. over 6 h. NH₄Cl saturated solution was added and the mixture was extracted with EtOAc twice.

The combined organic phases were dried over sodium sulfate and concentrated under vacuum. The crude was purified by FC eluent EtOAc/cyclohexane 0% to 20% to give 1-(4,4′-bis(benzyloxy)-5,5′-diethyl-2′-fluoro-[1,1′-biphenyl]-2-yl)ccyclobutan-1-ol (80 mg, 0.16 mmol, 34%). The next step where the desired product could eventually be isolated. R_{f} 0.3 (EA/cyclohexane 20%) ¹H NMR (400 MHz, CDCl₃) δ 7.58-7.28 (m, 10H), 7.08 (d, J=8.9 Hz, 1H), 6.97 (s, 1H), 6.89 (s, 1H), 6.68 (d, J=11.7 Hz, 1H), 5.13 (s, 2H), 5.08 (d, J=2.1 Hz, 2H), 2.70 (dq, J=9.9, 7.5 Hz, 4H), 2.26 (m, 2H), 2.06 (m, 3H), 1.85-1.71 (m, 1H), 1.22 (td, J=7.5, 5.6 Hz, 5H).

### Step 3: Synthesis of 3,8-bis(benzyloxy)-9-ethyl-2-methylspiro[benzo[c]chromene-6,1′-cyclobutane]

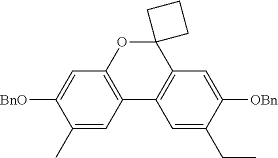 [SMILES: CCc1cc2c(cc1OCc1ccccc1)C1(CCC1)Oc1cc(OCc3ccccc3)c(C)cc1-2]

Sodium hydride (46 mg, 60% Wt, 5 Eq, 1.16 mmol) was added to a solution of 1-(4,4′-bis(benzyloxy)-5-ethyl-2′-fluoro-5′-methyl-[1,1′-biphenyl]-2-yl) cyclobutan-1-ol (115 mg, 1 Eq, 232 μmol) in DMF (3 mL) at 120° C. and stirring continued for 15 min at 120° C. The mixture was cooled down to room temperature, NH4Cl saturated solution was added and the aqueous phase was extracted with EtOAc twice. The combined organic phases were dried over sodium sulfate and evaporated under vacuum. The crude was purified by FC eluent EtOAc/cyclohexane 0% to 10% to give 3,8-bis(benzyloxy)-9-ethyl-2-methylspiro[benzo[c]chromene-6,1′-cyclobutane] (90 mg, 0.19 mmol, 82%) as colorless oil. R_{f} 0.5(EA/cyclohexane 10%). ¹H NMR (400 MHz, CDCl₃) δ 7.53-7.27 (m, 12H), 6.94 (s, 1H), 6.60 (s, 1H), 5.17 (s, 2H), 5.08 (s, 2H), 2.76 (q, J=7.5 Hz, 2H), 2.53 (ddd, J=13.0, 10.0, 8.5 Hz, 2H), 2.36 (ddd, J=13.1, 8.8, 4.2 Hz, 1H), 2.28 (s, 3H), 2.01 (dt, J=15.6, 5.2 Hz, 1H), 1.74 (dt, J=11.3, 8.7 Hz, 1H), 1.28 (t, J=7.5 Hz, 3H).

### Step 4: Synthesis of 9-ethyl-2-methylspiro[benzo[c]chromene-6,1′-cyclobutane]-3,8-diol (10)

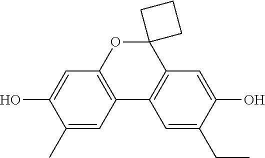 [SMILES: CCc1cc2c(cc1O)C1(CCC1)Oc1cc(O)c(C)cc1-2]

3,8-bis(benzyloxy)-9-ethyl-2-methylspiro[benzo[c]chromene-6,1′-cyclobutane] (80.00 mg, 1.00 Eq, 167.8 μmol) was dissolved in MeOH (4 mL). Nickelous chloride hexahydrate (199.5 mg, 5 Eq, 839.2 μmol) was added. SodiumTetrahydroborate (63.50 mg, 59.40 μL, 10 Eq, 1.678 mmol) were added portionwise (gaz evolution) until consumption of all starting material. TLC showed no more starting material. The mixture was filtered off and the crude was loaded on silica gel and purified by FC eluent MeOH/DCM 0% to 4% to give 9-ethyl-2-methylspiro[benzo[c]chromene-6,1′-cyclobutane]-3,8-diol (40 mg, 0.13 mmol, 80%) as a white solid. R_{f} 0.4 (MeOH/DCM 4%). ¹H NMR (400 MHz, DMSO) δ 9.35 (s, 1H), 9.32 (s, 1H), 7.40 (s, 1H), 7.37 (s, 1H), 6.87 (s, 1H), 6.38 (s, 1H), 2.58 (t, J=7.5 Hz, 2H), 2.39 (ddd, J=12.7, 10.0, 8.6 Hz, 2H), 2.23 (ddd, J=12.7, 8.7, 4.0 Hz, 2H), 2.09 (s, 3H), 1.95 (ddd, J=20.6, 9.7, 4.4 Hz, 1H), 1.74 (dt, J=11.0, 8.6 Hz, 1H), 1.16 (t, J=7.5 Hz, 3H). MS: m/z: 297 [M+H]⁺.

### 17. Synthesis of 2-ethyl-9-methylspiro[benzo[c]chromene-6,1′-cyclobutane]-3,8-diol (11)

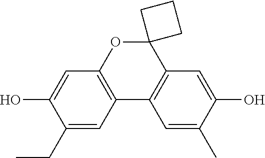 [SMILES: CCc1cc2c(cc1O)OC1(CCC1)c1cc(O)c(C)cc1-2]

11 was prepared from 1-(benzyloxy)-5-bromo-4-iodo-2-methylbenzene) and 2-(4-(benzyloxy)-5-ethyl-2-fluorophenyl)-4,4,5,5-tetramethyl-1,3,2-dioxaborolane in 4 steps.

### Synthesis of 2-(4-(benzyloxy)-5-ethyl-2-fluorophenyl)-4,4,5,5-tetramethyl-1,3,2-dioxaborolane

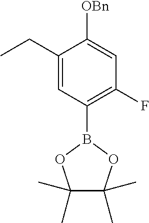 [SMILES: CCc1cc(B2OC(C)(C)C(C)(C)O2)c(F)cc1OCc1ccccc1]

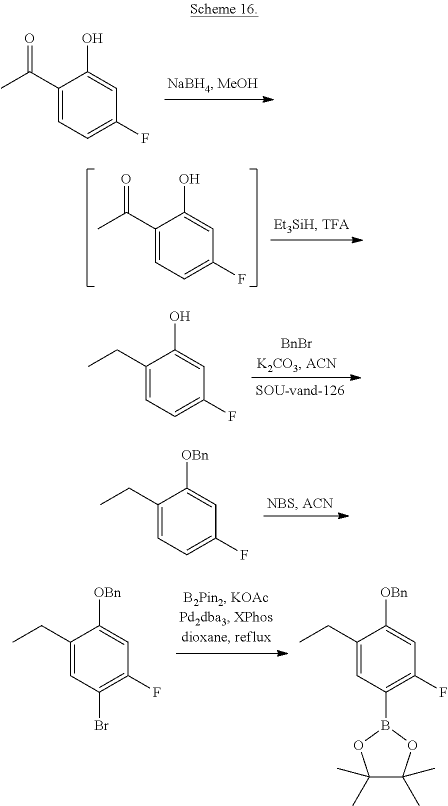

### Step 1: Synthesis of 2-ethyl-5-fluorophenol

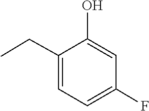

Step a —NaBH₄ (245 mg, 1 Eq, 6.49 mmol) was added portion wise to a stirred solution of 1-(4-fluoro-2-hydroxyphenyl)ethan-1-one (1.00 g, 1 Eq, 6.49 mmol) in methanol (24 g, 30 mL, 1.1e+2 Eq, 0.74 mol) at 0° C. over 30 minutes. The reaction mixture was stirred at room temperature for 15 hours. Then the reaction mixture was quenched with NH4Cl. The residue was diluted with ethyl acetate. The organic extracts were washed with water, brine, dried over anhydrous sodium sulphate and evaporated to afford crude 5-fluoro-2-(1-hydroxyethyl)phenol was taken for next step without any purification.

Step b—TFA (7.40 g, 5.00 mL, 10 Eq, 64.9 mmol) was added dropwise to a solution of 5-fluoro-2-(1-hydroxyethyl)phenol(intermediate) (4 g), triethylsilane (1.51 g, 2.07 mL, 2 Eq, 13.0 mmol) in 15 mL of dichloromethane at 0° C. The reaction mixture was stirred at room temperature for 15 hour. Then the reaction mixture was evaporated under vacuum ad the crude was extracted with Na2CO3 sat sol and EtOAc twice. The organic solvent was dried over sodium sulfate and evaporated under vacuum. The crude was purified by FC eluent EtOAc/cyclohexan 0% to 7% to give 5-bromo-2-ethylphenol (2.2 g, 11 mmol, 55%) as yellowish oil which crystalizes at r.t. ¹H NMR (400 MHz, CDCl₃) δ 7.11-6.99 (m, 1H), 6.59 (td, J=8.4, 2.5 Hz, 1H), 6.52 (dd, J=9.9, 2.5 Hz, 1H), 4.09 (s, 1H), 2.59 (q, J=7.5 Hz, 2H), 1.22 (t, J=7.5 Hz, 3H).

### Step 2: Synthesis of 2-(benzyloxy)-1-ethyl-4-fluorobenzene

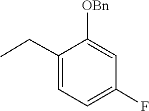 [SMILES: CCc1ccc(F)cc1OCc1ccccc1]

In a 50 mL round bottom flask, 2-ethyl-5-fluorophenol (2000 mg, 1.00 Eq, 14.27 mmol) was dissolved in dry acetonitrile (585.8 mg, 71.35 mL, 0.200 molar, 1 Eq, 14.27 mmol) at room temperature. potassium carbonate (3.944 g, 2.00 Eq, 28.54 mmol) was added then benzyl bromide (2.197 g, 1.528 mL, 0.9 Eq, 12.84 mmol) was added dropwise at room temperature. The suspension was stirred overnight at room temperature. Water was added and the aqueous phase was extracted with EA twice, washed with water and dried over sodium sulfate. The solvent was concentrated under vacuum to give 2-(benzyloxy)-1-ethyl-4-fluorobenzene (3.5 g, 15 mmol, 110%) as yellowish oil. ¹H NMR (400 MHz, CDCl₃) δ 7.47-7.30 (m, 7H), 7.10 (dd, J=8.2, 6.9 Hz, 1H), 6.71-6.56 (m, 2H), 5.06 (s, 2H), 2.71-2.60 (m, 2H), 1.20 (t, J=7.5 Hz, 3H).

### Step 3: Synthesis of 1-(benzyloxy)-4-bromo-2-ethyl-5-fluorobenzene

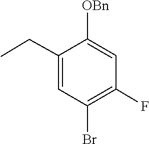 [SMILES: CCc1cc(Br)c(F)cc1OCc1ccccc1]

NBS (1.333 g, 1.15 Eq, 7.491 mmol) was added to a solution of 2-(benzyloxy)-1-ethyl-4-fluorobenzene (1.500 g, 1.00 Eq, 6.514 mmol) in MeCN (267.4 mg, 21.71 mL, 0.300 molar, 1 Eq, 6.514 mmol) at r.t. and the reaction mixture was stirred o.n. NaOH 1M (30 mL) was added at room temperature and the aqueous phase was extracted with EA twice. The combined organic phases were washed successively with water and brine and dried over sodium sulfate then concentrated under vacuum to give 1-(benzyloxy)-4-bromo-2-ethyl-5-fluorobenzene (1.70 g, 5.50 mmol, 84.4%) as yellowish oil. ¹H NMR (400 MHz, CDCl₃) δ 7.45-7.39 (m, 5H), 7.32-7.27 (m, 1H), 6.70 (d, J=10.4 Hz, 1H), 5.04 (s, 2H), 2.75-2.59 (m, 2H), 1.20 (t, J=7.5 Hz, 3H).

### Step 4: Synthesis of 2-(4-(benzyloxy)-5-ethyl-2-fluorophenyl)-4,4,5,5-tetramethyl-1,3,2-dioxaborolane

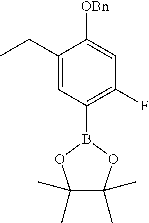 [SMILES: CCc1cc(B2OC(C)(C)C(C)(C)O2)c(F)cc1OCc1ccccc1]

1-(benzyloxy)-4-bromo-2-ethyl-5-fluorobenzene (1.70 g, 1 Eq, 5.50 mmol), 4,4,4′,4′,5,5,5′,5′-octamethyl-2,2′-bi(1,3,2-dioxaborolane) (2.51 g, 1.8 Eq, 9.90 mmol), potassium acetate (2.16 g, 4 Eq, 22.0 mmol) were added followed by dioaxane (60 mL)l. The mixture was stirred at 100° C. for 12 hours under N₂. After cooling to 30° C., the reaction mixture was filtered through Celite. The filtrate was concentrated under reduced pressure. Water (300 mL) was added and the aqueous layer was extracted with EtOAc (300 mL×3). The combined organic layer was washed with brine (300 mL), dried over anhydrous Na₂SO₄, filtered and concentrated to give the crude product. The crude product was purified by flash chromatography on silica gel (EtOAc in petroleum ether=0-10%) to give the 2-(4-(benzyloxy)-5-ethyl-2-fluorophenyl)-4,4,5,5-tetramethyl-1,3,2-dioxaborolane (1.1 g, 3.1 mmol, 56%) as yellowish oil. ¹H NMR (400 MHz, CDCl₃) δ 7.49 (d, J=7.1 Hz, 1H), 7.43-7.32 (m, 5H), 6.59 (d, J=11.2 Hz, 1H), 5.08 (s, 2H), 2.65 (q, J=7.5 Hz, 2H), 1.35 (s, 12H), 1.20 (t, J=7.5 Hz, 3H).

### 18. Synthesis of 2-ethyl-9-methylspiro[benzo[c]chromene-6,1′-cyclobutane]-3,8-diol (11)

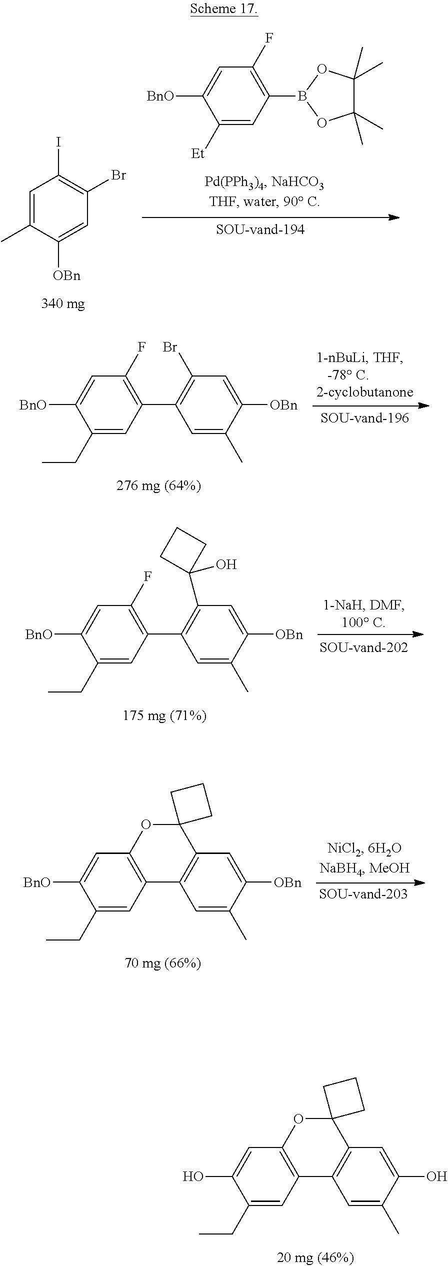 [SMILES: Cc1cc(I)c(Br)cc1OCc1ccccc1 ; CCc1cc(B2OC(C)(C)C(C)(C)O2)c(F)cc1OCc1ccccc1 ; CCc1cc(-c2cc(C)c(OCc3ccccc3)cc2Br)c(F)cc1OCc1ccccc1 ; CCc1cc(-c2cc(C)c(OCc3ccccc3)cc2C2(O)CCC2)c(F)cc1OCc1ccccc1 ; CCc1cc2c(cc1OCc1ccccc1)OC1(CCC1)c1cc(OCc3ccccc3)c(C)cc1-2 ; CCc1cc2c(cc1O)OC1(CCC1)c1cc(O)c(C)cc1-2]

### Step 1: Synthesis of 4,4′-bis(benzyloxy)-2-bromo-5′-ethyl-2′-fluoro-5-methyl-1,1′-biphenyl

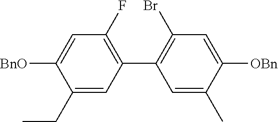 [SMILES: CCc1cc(-c2cc(C)c(OCc3ccccc3)cc2Br)c(F)cc1OCc1ccccc1]

1-(benzyloxy)-5-bromo-4-iodo-2-methylbenzene (340 mg, 1 Eq, 844 μmol) and 2-(4-(benzyloxy)-5-ethyl-2-fluorophenyl)-4,4,5,5-tetramethyl-1,3,2-dioxaborolane (421 mg, 1.4 Eq, 1.18 mmol) were dissolved in dioxane (10 mL). Bis-(triphenylphosphino)-palladous chloride (59.2 mg, 0.1 Eq, 84.4 μmol) was added and the solution was degassed for 5 min. then sodium bicarbonate (213 mg, 2.53 mL, 1 molar, 3 Eq, 2.53 mmol) was added dropwise and the mixture was heated at 90° C. overnight. Water was added and the mixture was extracted with EtOAc twice. The combined organic phases were dried over sodium sulfate and concentrated under vacuum. The crude was purified by FC biotage EtOAc/cyclohexane 0% to 1% to 2% to 3% to 4% 5% to give 4,4′-bis(benzyloxy)-2-bromo-5′-ethyl-2′-fluoro-5-methyl-1,1′-biphenyl (276 mg, 546 μmol, 64.7%) as colorless oil. R_{f}0.3 (EA/cyclohexane 2%). ¹H NMR (400 MHz, CDCl₃) δ 7.54-7.39 (m, 10H), 7.18 (s, 1H), 7.11 (t, J=0.8 Hz, 1H), 7.05 (d, J=8.6 Hz, 1H), 6.71 (d, J=11.5 Hz, 1H), 5.09 (s, 4H), 2.70 (qd, J=7.5, 2.1 Hz, 2H), 2.25 (d, J=0.8 Hz, 3H), 1.27-1.18 (m, 3H).

### Step 2: Synthesis of 4,4′-bis(benzyloxy)-2-bromo-5′-ethyl-2′-fluoro-5-methyl-1,1′-biphenyl

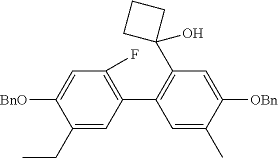 [SMILES: CCc1cc(-c2cc(C)c(OCc3ccccc3)cc2C2(O)CCC2)c(F)cc1OCc1ccccc1]

nBuLi (94.3 mg, 920 μL, 1.6 molar, 3 Eq, 1.47 mmol) was added to a solution of 4,4′-bis(benzyloxy)-2-bromo-5′-ethyl-2′-fluoro-5-methyl-1,1′-biphenyl (248 mg, 1 Eq, 491 μmol) in THF (7 mL) at −78° C. The mixture was stirred for 30 min at −78° C. then 1-Oxocyclobutane (344 mg, 367 μL, 10 Eq, 4.91 mmol) was added dropwise and the mixture was allowed to warm to rt. over 6 h. NH4Cl saturated solution was added and the mixture was extracted with EtOAc twice. The combined organic phases were dried over sodium sulfate and concentrated under vacuum. The crude was purified by FC eluent EtOAc/cyclohexane 0% to 20% to give 1-(4,4′-bis(benzyloxy)-5′-ethyl-2′-fluoro-5-methyl-[1,1′-biphenyl]-2-yl)cyclobutan-1-ol (175 mg, 352 μmol, 71.8%). R_{f} 0.3 EtOAc/cyclohexane 20%. ¹H NMR (400 MHz, CDCl₃) δ 7.58-7.31 (m, 10H), 7.06 (d, J=8.9 Hz, 1H), 6.96 (d, J=0.9 Hz, 1H), 6.88 (s, 1H), 6.67 (d, J=11.7 Hz, 1H), 5.13 (s, 2H), 5.10-4.97 (m, 2H), 2.68 (q, J=7.5 Hz, 2H), 2.28 (d, J=0.7 Hz, 3H), 2.04-1.92 (m, 2H), 1.60 (d, J=3.6 Hz, 1H), 1.39-1.32 (m, 1H), 1.21 (t, J=7.5 Hz, 3H). ¹⁹FNMR (400 MHz, CDCl₃) δ −115.97.

### Step 3: Synthesis of 3,8-bis(benzyloxy)-2-ethyl-9-methylspiro[benzo[c]chromene-6,1′-cyclobutane]

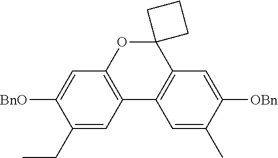 [SMILES: CCc1cc2c(cc1OCc1ccccc1)OC1(CCC1)c1cc(OCc3ccccc3)c(C)cc1-2]

Sodium hydride (0.18 g, 60% Wt, 20 Eq, 4.43 mmol) was added to a solution of 1-(4,4′-bis(benzyloxy)-5′-ethyl-2′-fluoro-5-methyl-[1,1′-biphenyl]-2-yl)cyclobutan-1-ol (110 mg, 1 Eq, 221 μmol) in DMF (3 mL) at 120° C. and stirring continued for 15 min at 120° C. The mixture was cooled down to room temperature, NH4Cl saturated solution was added and the aqueous phase was extracted with EtOAc twice. The combined organic phases were dried over sodium sulfate and evaporated under vacuum. The crude was purified by FC eluent EtOAc/cyclohexane 0% to 10% to give 3,8-bis(benzyloxy)-2-ethyl-9-methylspiro[benzo[c]chromene-6,1′-cyclobutane] (70 mg, 0.15 mmol, 66%) as a white solid. R_{f} 0.5. (EA/cyclohexane 10%). ¹H NMR (400 MHz, CDCl₃) δ 7.56-7.27 (m, 12H), 6.93 (s, 1H), 6.60 (s, 1H), 5.17 (s, 2H), 5.07 (s, 2H), 2.70 (q, J=7.5 Hz, 2H), 2.60-2.45 (m, 2H), 2.39-2.35 (m, 2H), 2.35 (d, J=0.7 Hz, 3H), 2.09-1.90 (m, 1H), 1.74 (dp, J=11.4, 8.7 Hz, 1H), 1.26 (t, J=7.5 Hz, 3H).

### Step 4: Synthesis of 2-ethyl-9-methylspiro[benzo[c]chromene-6,1′-cyclobutane]-3,8-diol (11)

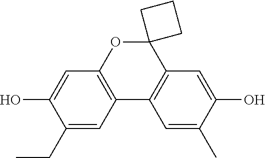 [SMILES: CCc1cc2c(cc1O)OC1(CCC1)c1cc(O)c(C)cc1-2]

3,8-bis(benzyloxy)-2-ethyl-9-methylspiro[benzo[c]chromene-6,1′-cyclobutane] (70.00 mg, 1.00 Eq, 146.9 μmol) was dissolved in MeOH (4 mL). Nickelous chloride hexahydrate (174.5 mg, 5 Eq, 734.3 μmol) was added. SodiumTetrahydroborate (55.56 mg, 51.97 μL, 10 Eq, 1.469 mmol) were added portionwise (gaz evolution). TLC showed no more starting material. The mixture was filtered off and the crude was loaded on silica gel and purified by FC eluent MeOH/DCM 0% to 4% to give 2-ethyl-9-methylspiro[benzo[c]chromene-6,1′-cyclobutane]-3,8-diol (20 mg, 67 μmol, 46%) as a white solid. R_{f} 0.4 MeOH/DCM 4%. ¹H NMR (400 MHz, DMSO) δ 9.33 (s, 2H), 7.41 (s, 1H), 7.37 (s, 1H), 6.87 (s, 1H), 6.38 (s, 1H), 2.39 (q, J=10.0 Hz, 2H), 2.27-2.19 (m, 2H), 2.19-2.12 (m, 3H), 2.02-1.90 (m, 1H), 1.83-1.67 (m, 1H), 1.14 (t, J=7.5 Hz, 3H). MS: m/z: 297 [M+H].

### 19. Synthesis of 2,9-diethylspiro[benzo[c]chromene-6,1′-cyclobutane]-3,8-diol (12)

13 was prepared from 1-(benzyloxy)-5-bromo-2-ethyl-4-iodobenzene and 2-(4-(benzyloxy)-5-ethyl-2-fluorophenyl)-4,4,5,5-tetramethyl-1,3,2-dioxaborolane in 4 steps.

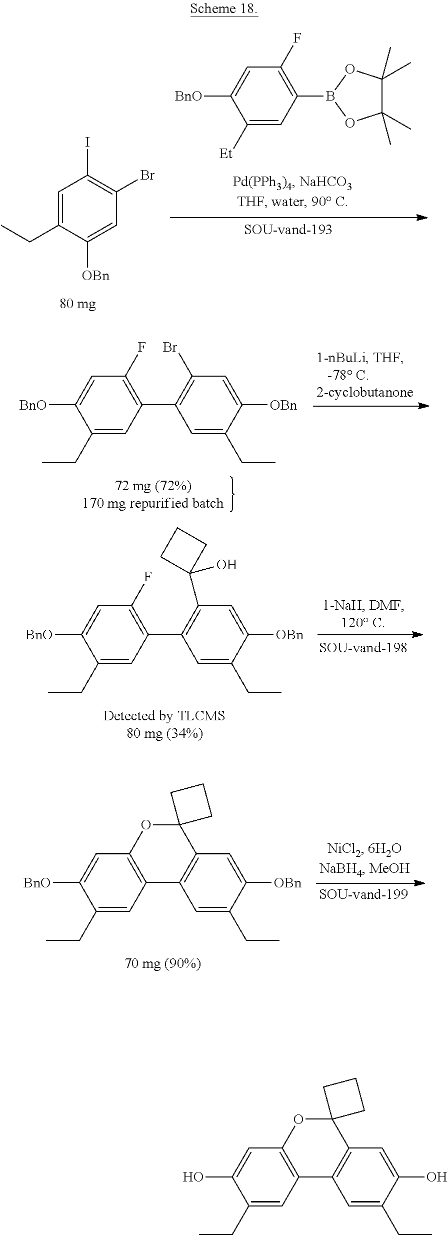 [SMILES: CCc1cc(I)c(Br)cc1OCc1ccccc1 ; CCc1cc(B2OC(C)(C)C(C)(C)O2)c(F)cc1OCc1ccccc1 ; CCc1cc(-c2cc(CC)c(OCc3ccccc3)cc2Br)c(F)cc1OCc1ccccc1 ; CCc1cc(-c2cc(CC)c(OCc3ccccc3)cc2C2(O)CCC2)c(F)cc1OCc1ccccc1 ; CCc1cc2c(cc1OCc1ccccc1)OC1(CCC1)c1cc(OCc3ccccc3)c(CC)cc1-2 ; CCc1cc2c(cc1O)OC1(CCC1)c1cc(O)c(CC)cc1-2]

### Step 1: Synthesis of 4,4′-bis(benzyloxy)-2-bromo-5,5′-diethyl-2′-fluoro-1,1′-biphenyl

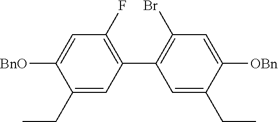 [SMILES: CCc1cc(-c2cc(CC)c(OCc3ccccc3)cc2Br)c(F)cc1OCc1ccccc1]

1-(benzyloxy)-5-bromo-2-ethyl-4-iodobenzene (380 mg, 1 Eq, 911 μmol) and 2-(4-(benzyloxy)-5-ethyl-2-fluorophenyl)-4,4,5,5-tetramethyl-1,3,2-dioxaborolane (454 mg, 1.4 Eq, 1.28 mmol) were dissolved in dioxane (10 mL). Bis-(triphenylphosphino)-palladous chloride (63.9 mg, 0.1 Eq, 91.1 μmol) was added and the solution was degassed for 5 min. then sodium bicarbonate (230 mg, 2.73 mL, 1 molar, 3 Eq, 2.73 mmol) was added dropwise and the mixture was heated at 90° C. overnight. Water was added and the mixture was extracted with EtOAc twice. The combined organic phases were dried over sodium sulfate and concentrated under vacuum. The crude was purified by FC biotage EtOAc/cyclohexane 0% to 1% to 2% to 5% to give 4,4′-bis(benzyloxy)-2-bromo-5,5′-diethyl-2′-fluoro-1,1′-biphenyl (375 mg, 722 μmol, 79.2%) as colorless oil. R_{f} 0.3 (EA/cyclohexane 2%). ¹H NMR (400 MHz, CDCl₃) δ 7.57-7.31 (m, 10H), 7.19 (s, 1H), 7.11 (d, J=0.7 Hz, 1H), 7.06 (d, J=8.7 Hz, 1H), 6.71 (d, J=11.5 Hz, 1H), 5.09 (s, 4H), 2.74-2.63 (m, 4H), 1.31-1.18 (m, 6H).

### Step 2: Synthesis of 1-(4,4′-bis(benzyloxy)-5,5′-diethyl-2′-fluoro-[1,1′-biphenyl]-2-yl)cyclobutan-1-ol

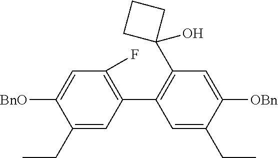 [SMILES: CCc1cc(-c2cc(CC)c(OCc3ccccc3)cc2C2(O)CCC2)c(F)cc1OCc1ccccc1]

nBuLi (53.28 mg, 519.78 μL, 1.6 molar, 1.8 Eq, 831.65 μmol) was added to a solution of 4,4′-bis(benzyloxy)-2-bromo-5,5′-diethyl-2′-fluoro-1,1′-biphenyl (240.00 mg, 1 Eq, 462.03 μmol) in THF (7 mL) at −78° C. The mixture was stirred for 30 min at −78° C. then 1-Oxocyclobutane (161.9 mg, 173 μL, 5 Eq, 2.3101 mmol) was added dropwise and the mixture was allowed to warm to rt. over 6 h. NH4Cl saturated solution was added and the mixture was extracted with EtOAc twice. The combined organic phases were dried over sodium sulfate and concentrated under vacuum. The crude was purified by FC eluent EtOAc/cyclohexane 0% to 20% to give 1-(4,4′-bis(benzyloxy)-5,5′-diethyl-2′-fluoro-[1,1′-biphenyl]-2-yl)cyclobutan-1-ol (80 mg, 0.16 mmol, 34%). As a yellowish oil. R_{f} 0.3 (EA/cyclohexane 20%). ¹H NMR (400 MHz, CDCl₃) δ 7.58-7.28 (m, 10H), 7.08 (d, J=8.9 Hz, 1H), 6.97 (s, 1H), 6.89 (s, 1H), 6.68 (d, J=11.7 Hz, 1H), 5.13 (s, 2H), 5.08 (d, J=2.1 Hz, 2H), 2.70 (dq, J=9.9, 7.5 Hz, 4H), 2.26 (m, 2H), 2.06 (m, 3H), 1.85-1.71 (m, 1H), 1.22 (td, J=7.5, 5.6 Hz, 5H).

### Step 3: Synthesis of 3,8-bis(benzyloxy)-2,9-diethylspiro[benzo[c]chromene-6,1′-cyclobutane]

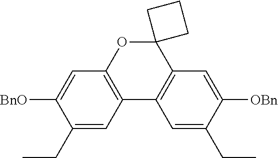 [SMILES: CCc1cc2c(cc1OCc1ccccc1)OC1(CCC1)c1cc(OCc3ccccc3)c(CC)cc1-2]

Sodium hydride (0.13 g, 60% Wt, 20 Eq, 3.1 mmol) was added to a solution of 1-(4,4′-bis(benzyloxy)-5,5′-diethyl-2′-fluoro-[1,1′-biphenyl]-2-yl)cyclobutan-1-ol (80 mg, 1 Eq, 0.16 mmol) in DMF (3 mL) at 120° C. and stirring continued for 15 min at 120° C. The mixture was cooled down to room temperature, NH4Cl saturated solution was added and the aqueous phase was extracted with EtOAc twice. The combined organic phases were dried over sodium sulfate and evaporated under vacuum. The crude was purified by FC eluent EtOAc/cyclohexane 0% to 10% to give 3,8-bis(benzyloxy)-2,9-diethylspiro[benzo[c]chromene-6,1′-cyclobutane] (70 mg, 0.14 mmol, 91%) as a white solid. R_{f} 0.5(EA/cyclohexane 10%). ¹H NMR (400 MHz, CDCl₃) δ 7.53-7.30 (m, 12H), 6.94 (s, 1H), 6.60 (s, 1H), 5.18 (s, 2H), 5.08 (s, 2H), 2.76 (q, J=7.6 Hz, 2H), 2.70 (q, J=7.6 Hz, 2H), 2.60-2.48 (m, 2H), 2.37 (dddd, J=13.1, 8.8, 4.2, 2.4 Hz, 2H), 2.05-1.94 (m, 1H), 1.75 (dt, J=11.4, 8.7 Hz, 1H), 1.36-1.16 (m, 6H).

### Step 4: Synthesis of 2,9-diethylspiro[benzo[c]chromene-6,1′-cyclobutane]-3,8-diol (12)

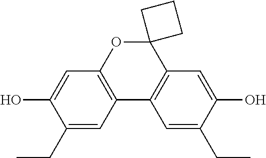 [SMILES: CCc1cc2c(cc1O)OC1(CCC1)c1cc(O)c(CC)cc1-2]

3,8-bis(benzyloxy)-2,9-diethylspiro[benzo[c]chromene-6,1′-cyclobutane] (70.00 mg, 1.00 Eq, 142.7 μmol) was dissolved in MeOH (4 mL). Nickelous chloride hexahydrate (169.5 mg, 5 Eq, 713.4 μmol) was added. Sodium Tetrahydroborate (53.97 mg, 50.49 μL, 10 Eq, 1.427 mmol) were added portion wise (gaz evolution). TLC showed no more starting material. The mixture was filtered off and the crude was loaded on silica gel and purified by FC eluent MeOH/DCM 0% to 4% to give 2,9-diethylspiro[benzo[c]chromene-6,1′-cyclobutane]-3,8-diol (29 mg, 93 μmol, 65%) as a white solid. R_{f} 0.4 (MeOH/DCM 4%). ¹H NMR (400 MHz, DMSO) δ 9.33 (s, 2H), 7.39 (d, J=1.7 Hz, 2H), 6.87 (s, 1H), 6.38 (s, 1H), 2.62-2.51 (m, 4H), 2.45-2.35 (m, 2H), 2.28-2.18 (m, 2H), 1.95 (ddt, J=15.3, 9.9, 4.7 Hz, 1H), 1.74 (dt, J=11.0, 8.6 Hz, 1H), 1.15 (q, J=7.4 Hz, 6H). MS: m/z: 311 [M+H]+.

### 20. Synthesis of 1′,6′-dimethylspiro[cyclopentane-1,9′-fluorene]-2′,4′,7′-triol (16)

16 was prepared from methyl 2-bromo-5-methoxy-4-methylbenzoate and 2-(4-(benzyloxy)-2-fluoro-5-methylphenyl)-4,4,5,5-tetramethyl-1,3,2-dioxaborolane in 4 steps.

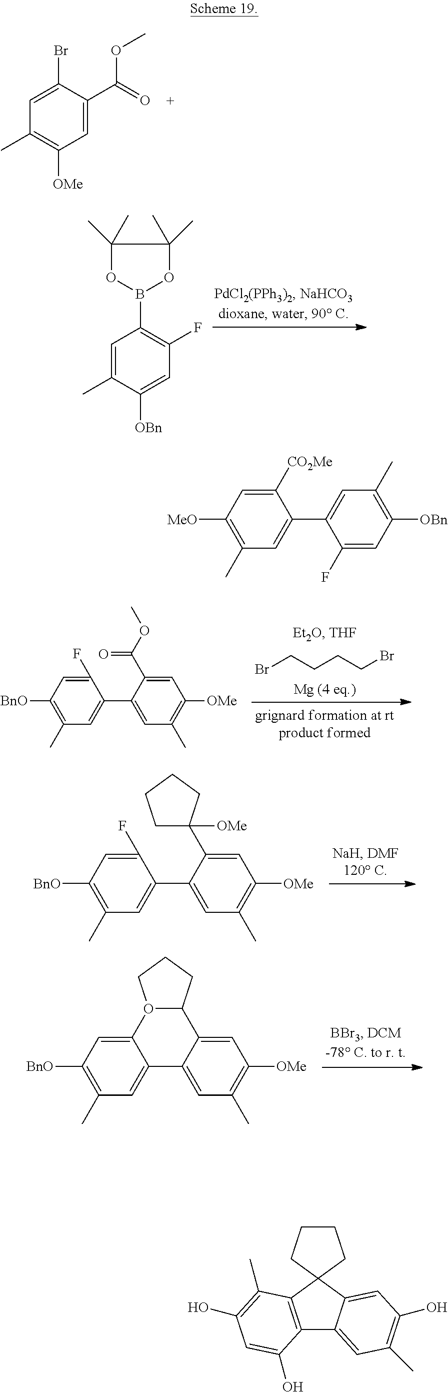

### Step 1: synthesis of methyl 4′-(benzyloxy)-2′-fluoro-4-methoxy-5,5′-dimethyl-[1,1′-biphenyl]-2-carboxylate

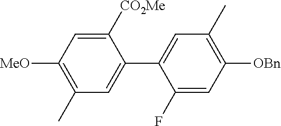 [SMILES: COC(=O)c1cc(OC)c(C)cc1-c1cc(C)c(OCc2ccccc2)cc1F]

Methyl 2-bromo-5-methoxy-4-methylbenzoate (370 mg, 1 Eq, 1.43 mmol), 2-(4-(benzyloxy)-2-fluoro-5-methylphenyl)-4,4,5,5-tetramethyl-1,3,2-dioxaborolane (635 mg, 1.3 Eq, 1.86 mmol) and Bis-(triphenylphosphino)-palladous chloride (100 mg, 0.1 Eq, 143 μmol) were suspended in THF (103 mg, 14.3 mL, 0.1 molar, 1 Eq, 1.43 mmol) and degassed with Nitrogen for 5 minutes. A solution of sodium bicarbonate (264 mg, 2.2 Eq, 3.14 mmol) in water (25.7 mg, 4.76 mL, 0.3 molar, 1 Eq, 1.43 mmol) was added at r.t. and the mixture was refluxed o.n. After cooling to room temperature, NH4Cl saturated solution was added and the aqueous phase was extracted with EA twice. The combined organic phases were dried over sodium sulfate, concentrated under vacuum and the organic residue was subjected to column chromatography biotage FC EA/cyclohexane 0% to 10% to give methyl 4′-(benzyloxy)-2′-fluoro-4-methoxy-5,5′-dimethyl-[1,1′-biphenyl]-2-carboxylate (480 mg, 1.22 mmol, 85.2%) as colourless oil. R_{f} 0.3 (EA/cyclohexane 10%). MS (APCI+): m/z=395.

### Step 2: synthesis of 1-(4′-(benzyloxy)-4-methoxy-3′,5-dimethyl-[1,1′-biphenyl]-2-yl)cyclopentan-1-ol

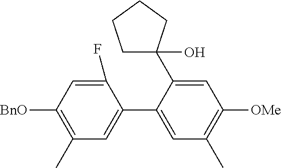 [SMILES: COc1cc(C2(O)CCCC2)c(-c2cc(C)c(OCc3ccccc3)cc2F)cc1C]

To a dry magnesium (22.18 mg, 3 Eq. 912.69 μmol) in dry diethyl ether (1.127 g, 1.58 mL, 50 Eq, 15.211 mmol) was added 1,4-dibromobutane (394.13 ng, 216.6 μL, 6 Eq, 1.8254 mmol) at n and the reaction mixture was stirred for 2 h. to this turbid solution was added a solution of methyl 4-(benzyloxy)-2fluoro-4-methoxy-5,5-dimenthyl-[1,1′-biphenyl]-2-carboxylate (120.00 ng, 1 Eq, 304.23 μmol) in dry TH-F (1 mL) and the reaction stirred for 5 h. tlc showed still some sm (a polar product was formed, TLC MS showed M-18). The reaction was quenched with NiCl and extracted with EtOAc. The combined organic phases were dried over sodium sulfate and evaporated under vacuum to give-(4′-(benzyloxy)-4-methoxy-3′,5-dimethyl-[1,1′biphenyl]-2-yl)cyclopentan-1-ol (120 mg. 298 μmol, 98.0%) crude used to the next step without further purification.

### Step 3: Synthesis of 3-(benzyloxy)-8-methoxy-2,9-dimethylspiro[benzo[c]chromene-6,1′-cyclopentane]

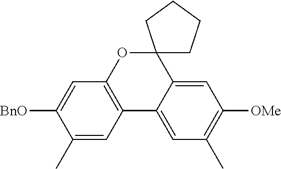 [SMILES: COc1cc2c(cc1C)-c1cc(C)c(OCc3ccccc3)cc1OC21CCCC1]

1-(4′-(benzyloxy)-2′-fluoro-4-methoxy-5,5′-dimethyl-[1,1′-biphenyl]-2-yl)cyclopentan-1-ol (120 mg, 1 Eq, 285 μmol) was dissolved in DMF (3 mL) and heated at 120° C. NaH (57 mg, 60% Wt, 5 Eq, 1.43 mmol) was added portion wise at 120° C. and stirring continued for 15 min at 120° C.

The mixture was cooled down to room temperature, NH4Cl saturated solution was added and the aqueous phase was extracted with EtOAc twice. The combined organic phases were dried over sodium sulfate and evaporated under vacuum. The crude was purified by FC eluent EtOAc/cyclohexane 0% to 10% to give 3-(benzyloxy)-8-methoxy-2,9-dimethylspiro[benzo[c]chromene-6,1′-cyclopentane] (40 mg, 0.10 mmol, 35%) as a white solid. R_{f} 0.5. (EA/cyclohexane 10%). MS (APCI+): m/z=401. ¹H NMR (400 MHz, CDCl₃) δ 7.48-7.28 (m, 7H), 6.67 (s, 1H), 6.52 (s, 1H), 5.06 (s, 2H), 3.86 (d, J=1.4 Hz, 3H), 2.26 (s, 6H), 2.22 (m, 2H), 2.03-1.88 (m, 4H), 1.82 (d, J=7.6 Hz, 2H).

### Step 4: Synthesis of 1′,6′-dimethylspiro[cyclopentane-1,9′-fluorene]-2′,4′,7′-triol (16)

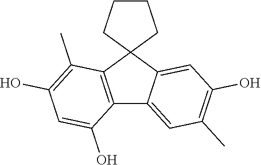 [SMILES: Cc1cc2c(cc1O)C1(CCCC1)c1c(C)c(O)cc(O)c1-2]

BBr₃ (65.68 mg, 262.2 μL, 1 molar, 3 Eq, 262.2 μmol) was added to a suspension of 3-(benzyloxy)-8-methoxy-2,9-dimethylspiro[benzo[c]chromene-6,1′-cyclopentane] (35.00 mg, 1 Eq, 87.39 μmol) in DCM (4.305 g, 3.261 mL, 580 Eq, 50.68 mmol) at −78° C. over 2 min and the mixture was allowed to warm to r.t o.n. The reaction mixture was quenched with NaHCO₃ saturated solution, and the aqueous phase was extracted with EtOAc. The combined organic phases were dried over sodium sulfate and the organic residue was subjected to column chromatography biotage FC MeOH/DCM 0% to 20% to give 1′,6′-dimethylspiro[cyclopentane-1,9′-fluorene]-2′,4′,7′-triol (15 mg, 51 μmol, 58%) as a white solid. R_{f} 0.5 MeOH/DCM 10%. ¹H NMR (400 MHz, DMSO) δ 9.22 (s, 1H), 8.98 (s, 1H), 8.88 (s, 1H), 7.47 (s, 1H), 6.80 (s, 1H), 6.33 (d, J=1.8 Hz, 1H), 2.34-2.23 (m, 2H), 2.11 (s, 3H), 2.10 (s, 3H), 2.07 (m, 4H), 1.75-1.56 (m, 2H). MS: m/z: 297 [M+H]+.

### 21. Synthesis of 2,9-dicyclopropylspiro[benzo[c]chromene-6,1′-cyclobutane]-3,8-diol (17)

17 was prepared from 1-(benzyloxy)-5-bromo-2-cyclopropyl-4-iodobenzene and 2-(4-(benzyloxy)-5-cyclopropyl-2-fluorophenyl)-4,4,5,5-tetramethyl-1,3,2-dioxaborolane in 4 steps:

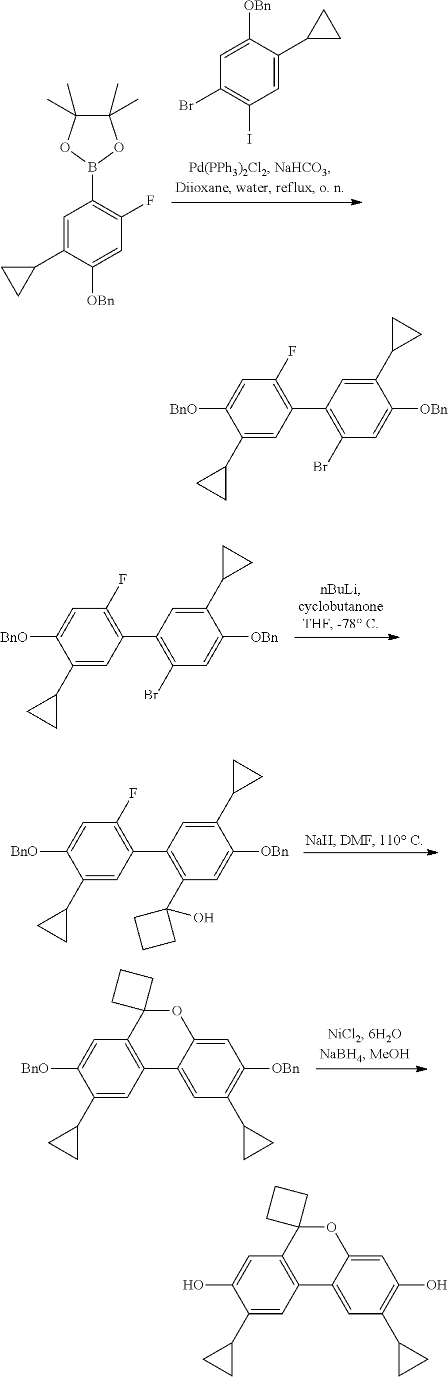

### Step 1: Synthesis of 4,4′-bis(benzyloxy)-2-bromo-5,5′-dicyclopropyl-2′-fluoro-1,1′-biphenyl

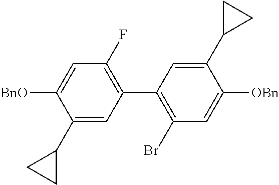 [SMILES: Fc1cc(OCc2ccccc2)c(C2CC2)cc1-c1cc(C2CC2)c(OCc2ccccc2)cc1Br]

1-(benzyloxy)-5-bromo-2-cyclopropyl-4-iodobenzene (340 mg, 1 Eq, 792 μmol) and 2-(4-(benzyloxy)-5-cyclopropyl-2-fluorophenyl)-4,4,5,5-tetramethyl-1,3,2-dioxaborolane (379 mg, 1.3 Eq, 1.03 mmol) were dissolved in dioxane (10 mL). Bis-(triphenylphosphino)-palladous chloride (55.6 mg, 0.1 Eq, 79.2 μmol) was added and the solution was degassed for 5 min, then sodium bicarbonate (200 mg, 2.38 mL, 1 molar, 3 Eq, 2.38 mmol) was dissolved in water (4 mL) and added dropwise and the mixture was heated at 90° C. 2 h. Water was added and the mixture was extracted with EtOAc twice. The combined organic phases were dried over sodium sulfate and concentrated under vacuum. The crude was purified by FC biotage EtOAc/cyclohexane 0% to 5% to give 4,4′-bis(benzyloxy)-2-bromo-5,5′-dicyclopropyl-2′-fluoro-1,1′-biphenyl (330 mg, 607 mol, 76.6%) as colourless oil. R_{f} 0.3 EtOAc/cycolohexane 2%. ¹H NMR (400 MHz, CDCl₃) δ 7.67-7.28 (m, 11H), 7.18 (s, 1H), 6.82 (dd, J=5.7, 3.8 Hz, 1H), 6.75 (d, J=3.9 Hz, 1H), 6.72 (d, J=6.0 Hz, 4H), 2.28-2.09 (m, 2H), 0.92 (dddd, J=7.4, 5.5, 4.4, 1.8 Hz, 4H), 0.69-0.60 (m, 4H). ¹⁹F NMR (376 MHz, CDCl₃) δ −116.77.

### Step 2: Synthesis of 1-(4,4′-bis(benzyloxy)-5,5′-dicyclopropyl-2′-fluoro-[1,1′-biphenyl]-2-yl)cyclobutan-1-ol

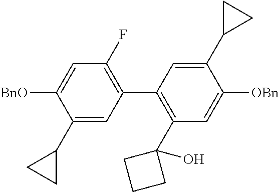 [SMILES: OC1(c2cc(OCc3ccccc3)c(C3CC3)cc2-c2cc(C3CC3)c(OCc3ccccc3)cc2F)CCC1]

nBuLi (99.01 mg, 966.00 L, 1.6 molar, 3 Eq, 1.5456 mmol) was added under nitrogen atmosphere, to a solution of 4,4′-bis(benzyloxy)-2-bromo-5,5′-dicyclopropyl-2′-fluoro-1,1′-biphenyl (280.00 mg, 1.00 Eq, 515.20 mol) in THF (7 mL) at −78° C. The mixture was stirred for 30 min at −78° C. then 1-Oxocyclobutane (361.1 mg, 386 L, 10 Eq, 5.1520 mmol) was added dropwise and the mixture was allowed to warm to rt. over 6 h. NH4Cl saturated solution was added and the mixture was extracted with EtOAc twice. The combined organic phases were dried over sodium sulfate and concentrated under vacuum. The crude was purified by FC eluent EtOAc/cyclohexane 0% to 20% to give 1-(4,4′-bis(benzyloxy)-5,5′-dicyclopropyl-2′-fluoro-[1,1′-biphenyl]-2-yl)cyclobutan-1-ol (85 mg, 0.16 mmol, 31%). R_{f} 0.3 EtOAc/cyclohexane 20%. ¹H NMR (400 MHz, CDCl₃) δ 7.76-7.30 (m, 10H), 6.88 (s, 1H), 6.75 (d, J=8.7 Hz, 1H), 6.67 (d, J=11.6 Hz, 1H), 6.61 (s, 1H), 5.15 (s, 2H), 5.11 (s, 2H), 2.34-2.13 (m, 2H), 2.11-1.91 (m, 2H), 1.84-1.68 (m, 2H), 1.68-1.30 (m, 2H), 1.03-0.80 (m, 4H), 0.75-0.56 (m, 4H). ¹⁹F NMR (376 MHz, CDCl₃) δ −117.

### Step 3: Synthesis of 3,8-bis(benzyloxy)-2,9-dicyclopropylspiro[benzo[c]chromene-6,1′-cyclobutane]

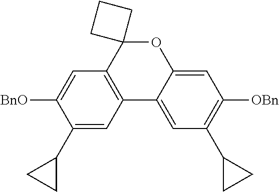 [SMILES: c1ccc(COc2cc3c(cc2C2CC2)-c2cc(C4CC4)c(OCc4ccccc4)cc2C2(CCC2)O3)cc1]

NaH (18 mg, 60% Wt, 3 Eq, 0.45 mmol) was added to a solution of 1-(4,4′-bis(benzyloxy)-5,5′-dicyclopropyl-2′-fluoro-[1,1′-biphenyl]-2-yl)cyclobutan-1-ol (80 mg, 1 Eq, 0.15 mmol) in DMF (7 mL) at 110° C. Stirring continued for 15 min at 110° C. TLC showed no more staring material. The reaction mixture was cooled down to room temperature. a solution of NaHCO₃ 1/2 saturated was added slowly and the aqueous phase was extracted with EtOAc twice. The combined organic phases were washed successively with water and brine and dried over sodium sulfate. solvent was evaporated and the crude was purified by FC eluent EtOAc/cyclohexane 0% to 5% to give 3,8-bis(benzyloxy)-2,9-dicyclopropylspiro[benzo[c]chromene-6,1′-cyclobutane] (65 mg, 0.13 mmol, 84%). R_{f} 0.7 EtOAc/cyclohexane 5%. ¹H NMR (400 MHz, CDCl₃) δ 7.53-7.31 (m, 10H), 7.13 (s, 1H), 7.09 (s, 1H), 6.93 (s, 1H), 6.60 (s, 1H), 5.20 (s, 2H), 5.11 (s, 2H), 2.57-2.44 (m, 2H), 2.42-2.31 (m, 2H), 2.26 (ddd, J=8.5, 5.3, 3.1 Hz, 1H), 2.15 (ddd, J=8.5, 5.4, 3.1 Hz, 1H), 2.05-1.92 (m, 1H), 1.73 (dt, J=11.4, 8.7 Hz, 1H), 1.05-0.82 (m, 4H), 0.80-0.61 (m, 4H).

### Step 4: Synthesis of 2,9-dicyclopropylspiro[benzo[c]chromene-6,1′-cyclobutane]-3,8-diol

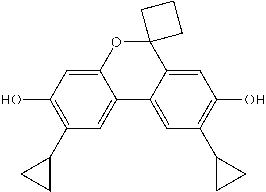 [SMILES: Oc1cc2c(cc1C1CC1)-c1cc(C3CC3)c(O)cc1C1(CCC1)O2]

3,8-bis(benzyloxy)-2,9-dicyclopropylspiro[benzo[c]chromene-6,1′-cyclobutane] (60.00 mg, 1.00 Eq, 116.6 mol) was dissolved in MeOH (4 mL). Nickelous chloride hexahydrate (138.5 mg, 5 Eq, 582.9 mol) was added. SodiumTetrahydroborate (88.20 mg, 82.51 L, 20 Eq, 2.332 mmol) were added portionwise (gaz evolution). TLC showed no more starting material. The mixture was filtered off and the crude was loaded on silica gel and purified by FC eluent MeOH/DCM 0% to 10% to give 2,9-dicyclopropylspiro[benzo[c]chromene-6,1′-cyclobutane]-3,8-diol (34 mg, 0.10 mmol, 87%) as a yellowish foam. R_{f} 0.4 MeOH/DCM 4%. MS: m/z: [M+H]+ 335. ¹H NMR (400 MHz, DMSO) δ 9.37 (s, 1H), 9.36 (s, 1H), 7.04 (s, 1H), 7.00 (s, 1H), 6.86 (s, 1H), 6.37 (s, 1H), 2.43-2.31 (m, 2H), 2.27-2.14 (m, 2H), 2.13-2.03 (m, 1H), 2.01-1.89 (m, 2H), 1.73 (dt, J=11.0, 8.5 Hz, 1H), 0.91-0.83 (m, 2H), 0.83-0.78 (m, 2H), 0.77-0.73 (m, 2H), 0.73-0.64 (m, 2H).

### 22. Synthesis of 2-cyclopropyl-9-methylspiro[benzo[c]chromene-6,1′-cyclobutane]-3,8-diol (18)

18 was prepared from 1-(benzyloxy)-5-bromo-4-iodo-2-methylbenzene and 2-(4-(benzyloxy)-5-cyclopropyl-2-fluorophenyl)-4,4,5,5-tetramethyl-1,3,2-dioxaborolane in 4 steps.

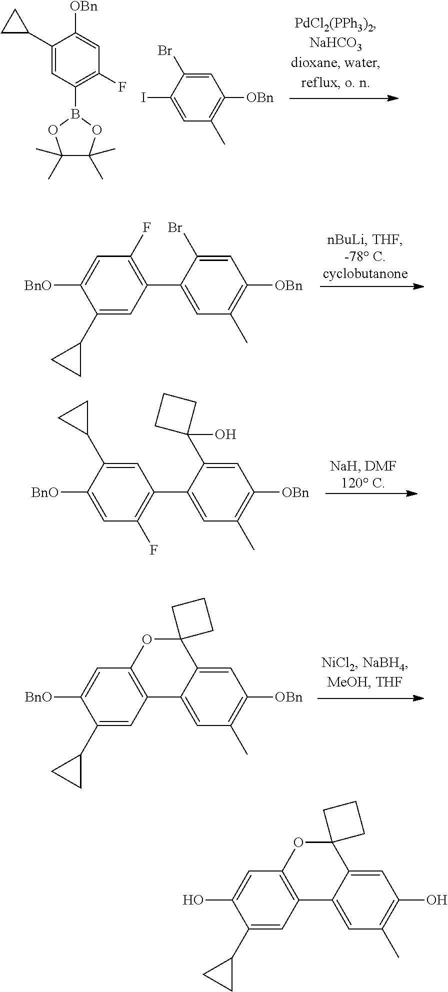

### Synthesis of 1-(benzyloxy)-5-bromo-4-iodo-2-methylbenzene

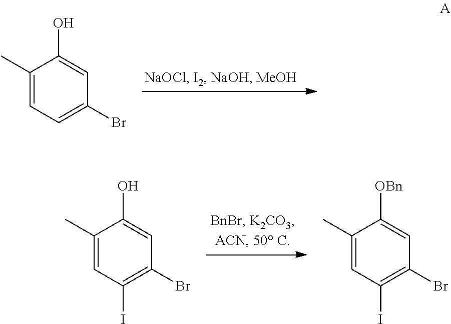

### Step 1: Synthesis of 5-bromo-4-iodo-2-methylphenol

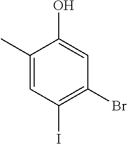

5-bromo-4-iodo-2-methylphenol was prepared from 5-bromo-2-ethylphenol according to procedure described in Beatrice Felber, Francois Dietrich, Helvetica, 2005, vol 88, 120-153.

### Step 2: Synthesis of 1-(benzyloxy)-5-bromo-4-iodo-2-methylbenzene

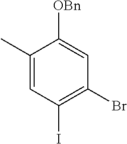 [SMILES: Cc1cc(I)c(Br)cc1OCc1ccccc1]

Benzyl Bromide (11.19 mL, 10.07 mmol, 1 eq.) was added to a mixture of 5-bromo-4-iodo-2-methylphenol (3.5 g, 11.18 mmol) and potassium carbonate (3.09 g, 22.37 mmol, 2 eq.) in ACN (60 mL) and the mixture was heated at 50° C. for 3 h. The reaction mixture was filtered off concentrated under vacuum give 1-(benzyloxy)-5-bromo-4-iodo-2-methylbenzene (4 g, 9.9 mmol, 89%) used to the next step without further purification. ¹H NMR (400 MHz, CDCl₃) δ 7.59 (d, J=0.9 Hz, 1H), 7.44-7.31 (m, 5H), 7.14 (s, 1H), 5.02 (s, 2H), 2.17 (s, 3H).

### Synthesis of 2-(4-(benzyloxy)-5-cyclopropyl-2-fluorophenyl)-4,4,5,5-tetramethyl-1,3,2-dioxaborolane

(4-(benzyloxy)-5-cyclopropyl-2-fluorophenyl)-4,4,5,5-tetramethyl-1,3,2-dioxaborolane was prepared from commercially available 2-bromo-5-fluorophenolin 4 steps:

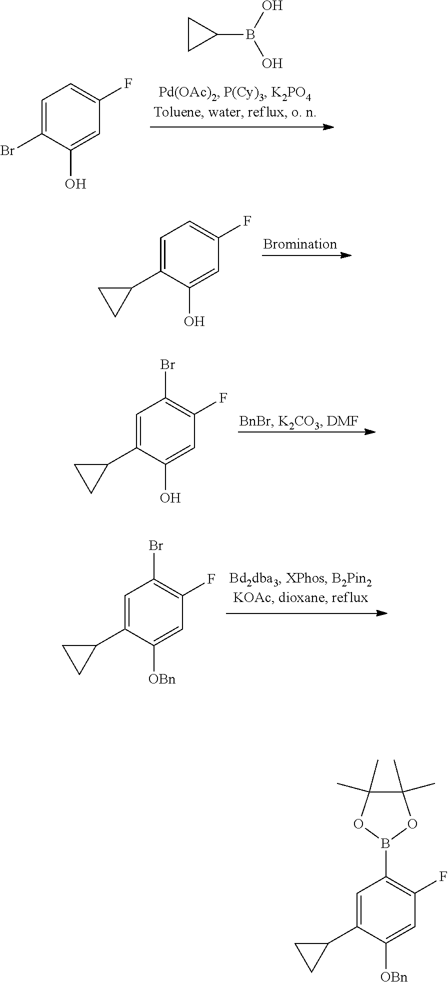 [SMILES: OB(O)C1CC1 ; Oc1cc(F)ccc1Br ; Oc1cc(F)ccc1C1CC1 ; Oc1cc(F)c(Br)cc1C1CC1 ; Fc1cc(OCc2ccccc2)c(C2CC2)cc1Br ; CC1(C)OB(c2cc(C3CC3)c(OCc3ccccc3)cc2F)OC1(C)C]

### Step 1: Synthesis of 2-cyclopropyl-5-fluorophenol

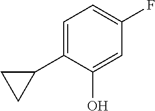 [SMILES: Oc1cc(F)ccc1C1CC1]

Phosphine, tricyclohexyl-(147 mg, 164 μL, 0.2 Eq, 524 μmol) and Palladium diacetate (58.8 mg, 0.1 Eq, 262 μmol) were suspended and stirred in degassed toluene (241 mg, 26.2 mL, 0.1 molar, 1 Eq, 2.62 mmol) for 5 min. 2-bromo-5-fluorophenol (500 mg, 292 μL, 1 Eq, 2.62 mmol) and cyclopropylboronic acid (899 mg, 4 Eq, 10.5 mmol) were added successively followed by Potassium phosphate, tribasic (3.33 g, 1.30 mL, 6 Eq, 15.7 mmol) dissolved in water (47.2 mg, 13.1 mL, 0.2 molar, 1 Eq, 2.62 mmol). The reaction mixture was heated at 110° C. overnight. The reaction mixture was extracted with EA and NH4Cl saturated solution. The combined organic phases were dried over sodium sulfate then concentrated under vacuum. The crude was purified by FC biotage EA/cyclohexane 0% to 35% to give 2-cyclopropyl-5-fluorophenol (340 mg, 2.23 mmol, 85.4%) as brownish oil. R_{f} 0.3 eluent EA/cyclohexane 10%. ¹H NMR (400 MHz, DMSO) δ 9.77 (s, 1H), 6.75 (dd, J=8.5, 6.8 Hz, 1H), 6.55 (dd, J=10.8, 2.7 Hz, 1H), 6.48 (td, J=8.6, 2.7 Hz, 1H), 1.98 (ddd, J=8.5, 4.9, 3.1 Hz, 1H), 0.86-0.77 (m, 2H), 0.62-0.42 (m, 2H).

### Step 2: Synthesis of 4-bromo-2-cyclopropyl-5-fluorophenol

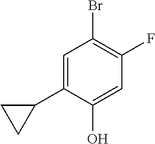 [SMILES: Oc1cc(F)c(Br)cc1C1CC1]

To a solution of 2-cyclopropyl-5-fluorophenol (2000 mg, 1 Eq, 13.14 mmol) in DCM (1.116 g, 65.72 mL, 0.2 molar, 1 Eq, 13.14 mmol) and methanol (421.1 mg, 43.81 mL, 0.3 molar, 1 Eq, 13.14 mmol) was added Tetra-n-butylammoniumTribromide (6.337 g, 1 Eq, 13.14 mmol) portion wise while stirring. The mixture was stirred at 25 0° C. for 3 hours. Water (250 mL) was added and the mixture was concentrated under reduced pressure. The aqueous layer was extracted with DCM (300 mL×3). The combined organic layers were washed with brine (600 mL), dried over anhydrous Na₂SO₄, filtered and concentrated to give the crude product which was purified by flash chromatography on silica gel eluent EtOAc/hexane 30% to give 4-bromo-2-cyclopropyl-5-fluorophenol (1.5 g, 6.5 mmol, 49%). ¹H NMR (400 MHz, DMSO) δ 10.11 (s, 1H), 6.97 (d, J=8.1 Hz, 1H), 6.71 (d, J=10.4 Hz, 1H), 1.98 (tt, J=8.4, 5.3 Hz, 1H), 0.94-0.71 (m, 2H), 0.71-0.48 (m, 2H).

### Step 3: Synthesis of 1-(benzyloxy)-4-bromo-2-cyclopropyl-5-fluorobenzene

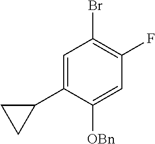 [SMILES: Fc1cc(OCc2ccccc2)c(C2CC2)cc1Br]

Benzyl bromide (1.110 g, 772.2 μL, 1 Eq, 6.492 mmol) was added to a suspension of 4-bromo-2-cyclopropyl-5-fluorophenol (1500 mg, 1 Eq, 6.492 mmol) and potassium carbonate (1.794 g, 2 Eq, 12.98 mmol) in acetonitrile (266.5 mg, 64.92 mL, 0.1 molar, 1 Eq, 6.492 mmol) at room temperature and the mixture was heated at 50° C. for 4 hours. The suspension was filtered off and the solvent evaporated under vacuum to give 1-(benzyloxy)-4-bromo-2-cyclopropyl-5-fluorobenzene (1.98 g, 6.16 mmol, 95.0%) as colourless. The compound was used without further purification. ¹H NMR (400 MHz, CDCl₃) δ 7.66-7.28 (m, 5H), 6.99 (dd, J=7.8, 0.6 Hz, 1H), 6.69 (d, J=10.4 Hz, 1H), 5.08 (s, 1H), 2.28-1.96 (m, 1H), 1.10-0.80 (m, 2H), 0.74-0.43 (m, 2H). ¹⁹F NMR (376 MHz, CDCl₃) δ −109.59.

### Step 4: Synthesis 2-(4-(benzyloxy)-5-cyclopropyl-2-fluorophenyl)-4,4,5,5-tetramethyl-1,3,2-dioxaborolane

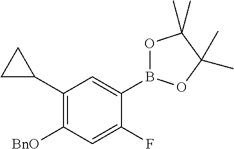 [SMILES: CC1(C)OB(c2cc(C3CC3)c(OCc3ccccc3)cc2F)OC1(C)C]

1-(benzyloxy)-4-bromo-2-cyclopropyl-5-fluorobenzene (500 mg, 1 Eq, 1.56 mmol), 4,4,4′,4′,5,5,5′,5′-octamethyl-2,2′-bi(1,3,2-dioxaborolane) (712 mg, 1.8 Eq, 2.80 mmol), potassium acetate (611 mg, 4 Eq, 6.23 mmol) were added followed by dioxane (60 mL). The mixture was stirred at 100° C. for 12 hours under N2. After cooling to 30° C., the reaction mixture was filtered through Celite. The filtrate was concentrated under reduced pressure. Water (300 mL) was added and the aqueous layer was extracted with EtOAc (300 mL×3). The combined organic layer was washed with brine, dried over anhydrous Na₂SO₄, filtered and concentrated to give the crude product. The crude product was purified by flash chromatography on silica gel (EtOAc in petroleum ether=0-30%) to give the 2-(4-(benzyloxy)-5-cyclopropyl-2-fluorophenyl)-4,4,5,5-tetramethyl-1,3,2-dioxaborolane (350 mg, 950 μmol, 61.1%) as colourless oil. ¹H NMR (400 MHz, CDCl₃) δ 7.50-7.31 (m, 5H), 7.24 (dd, J=7.1, 0.7 Hz, 1H), 6.59 (d, J=11.2 Hz, 1H), 5.11 (s, 2H), 2.15-2.01 (m, 1H), 0.98-0.80 (m, 2H), 0.76-0.56 (m, 2H). ¹⁹F NMR (376 MHz, CDCl₃) δ −103.82 (d, J=2.5 Hz).

### Synthesis of 18

Step 1: Synthesis of 4,4′-bis(benzyloxy)-2-bromo-5′-cyclopropyl-2′-fluoro-5-methyl-1,1′-biphenyl

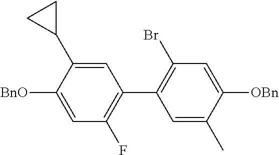 [SMILES: Cc1cc(-c2cc(C3CC3)c(OCc3ccccc3)cc2F)c(Br)cc1OCc1ccccc1]

1-(benzyloxy)-5-bromo-4-iodo-2-methylbenzene (300 mg, 1 Eq, 744 μmol) and 2-(4-(benzyloxy)-5-cyclopropyl-2-fluorophenyl)-4,4,5,5-tetramethyl-1,3,2-dioxaborolane (356 mg, 1.3 Eq, 968 μmol) were dissolved in dioxane (10 mL). Bis-(triphenylphosphino)-palladous chloride (52.2 mg, 0.1 Eq, 74.4 μmol) was added and the solution was degassed for 5 min. then sodium bicarbonate (188 mg, 2.23 mL, 1 molar, 3 Eq, 2.23 mmol) was dissolved in water (4 mL) and added dropwise and the mixture was heated at 90° C. 2 h. Water was added and the mixture was extracted with EtOAc twice. The combined organic phases were dried over sodium sulfate and concentrated under vacuum. The crude was purified by FC eluent EtOAc/cyclohexane 0% to 5% to give 4,4′-bis(benzyloxy)-2-bromo-5′-cyclopropyl-2′-fluoro-5-methyl-1,1′-biphenyl (350 mg, 676 μmol, 90.9%) as colourless oil. R_{f} 0.3 EtOAc/cycolohexane 2%. ¹H NMR (400 MHz, CDCl₃) δ 7.58-7.38 (m, 10H), 7.17 (s, 1H), 7.07 (t, J=0.8 Hz, 1H), 6.75 (d, J=8.4 Hz, OH), 5.12 (s, 1H), 5.09 (s, 1H), 2.24 (d, J=0.8 Hz, 3H), 2.20-2.11 (m, 1H), 0.95-0.84 (m, 2H), 0.68-0.55 (m, 2H).

### Step 2: Synthesis of 1-(4,4′-bis(benzyloxy)-5′-cyclopropyl-2′-fluoro-5-methyl-[1,1′-biphenyl]-2-yl)cyclobutan-1-ol

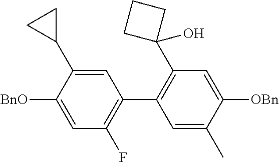 [SMILES: Cc1cc(-c2cc(C3CC3)c(OCc3ccccc3)cc2F)c(C2(O)CCC2)cc1OCc1ccccc1]

nBuLi (111 mg, 1.09 mL, 1.6 molar in THF, 3 Eq, 1.74 mmol) was added to a solution of 4,4′-bis(benzyloxy)-2-bromo-5′-cyclopropyl-2′-fluoro-5-methyl-1,1′-biphenyl (300 mg, 1 Eq, 580 μmol) in THF (7 mL) at −78° C. The mixture was stirred for 30 min at −78° C. then 1-Oxocyclobutane (406 mg, 434 μL, 10 Eq, 5.80 mmol) was added dropwise and the mixture was allowed to warm to rt. over 6 h. NH4Cl saturated solution was added and the mixture was twice with extracted. The combined organic phases were dried over sodium sulfate and concentrated under vacuum. The crude was purified by FC eluent EtOAc/cyclohexane 0% to 20% to give 1-(4,4′-bis(benzyloxy)-5′-cyclopropyl-2′-fluoro-5-methyl-[1,1′-biphenyl]-2-yl)cyclobutan-1-ol (140 mg, 275 μmol, 47.5%). R_{f} 0.3 EtOAc/cyclohexane 20%. ¹H NMR (400 MHz, CDCl₃) δ 7.56, 7.28 (m, 10H), 6.93 (d, J=0.9 Hz, 1H), 6.87 (s, 1H), 6.77 (d, J=8.7 Hz, 1H), 6.67 (d, J=11.6 Hz, 1H), 5.12 (d, J=3.2 Hz, 4H), 2.22-2.12 (m, 2H), 2.10-1.92 (m, 5H), 1.79-1.71 (m, 1H), 1.38-1.31 (m, 2H), 0.98-0.87 (m, 2H). MS (APCI+): m/z=508.2 found 491 (M-17).

### Step 3: Synthesis of 1-(4,4′-bis(benzyloxy)-5′-cyclopropyl-2′-fluoro-5-methyl-[1,1′-biphenyl]-2-yl)cyclobutan-1-ol

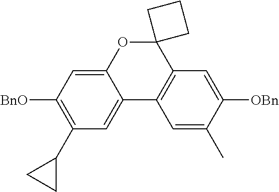 [SMILES: Cc1cc2c(cc1OCc1ccccc1)C1(CCC1)Oc1cc(OCc3ccccc3)c(C3CC3)cc1-2]

1-(4,4′-bis(benzyloxy)-5′-cyclopropyl-2′-fluoro-5-methyl-[1,1′-biphenyl]-2-yl)cyclobutan-1-ol (125 mg, 1 Eq, 246 μmol) was dissolved in DMF (1.5 mL) and heated to 110° C. NaH (29.5 mg, 5 Eq, 1.23 mmol) was added portion wise and stirring continued for 15 min. The reaction mixture was cooled down to room temperature and extracted with EtOAc and NaHCO₃ saturated solution. The organic phase was dried over sodium sulfate and concentrated under vacuum the crude was purified by FC eluent EtOAc/cyclohexane 0% to 30% to give 3,8-bis(benzyloxy)-2-cyclopropyl-9-methylspiro[benzo[c]chromene-6,1′-cyclobutane] (78 mg, 0.16 mmol, 65%) as colourless oil. R_{f} 0.6 EtOAc/cyclohexane 10%. ¹H NMR (400 MHz, CDCl₃) δ 7.57-7.27 (m, 13H), 7.16 (s, 1H), 6.92 (s, 1H), 6.60 (s, 1H), 5.17 (s, 2H), 5.11 (s, 20H), 2.66-2.47 (m, 2H), 2.46-2.21 (m, 5H), 2.17 (tt, J=8.5, 5.3 Hz, 1H), 2.07-1.92 (m, 1H), 1.73 (dt, J=11.4, 8.7 Hz, 1H), 1.01-0.86 (m, 2H), 0.80-0.68 (m, 2H).

### Step 4: Synthesis of 2-cyclopropyl-9-methylspiro[benzo[c]chromene-6,1′-cyclobutane]-3,8-diol

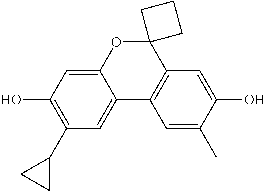 [SMILES: Cc1cc2c(cc1O)C1(CCC1)Oc1cc(O)c(C3CC3)cc1-2]

3,8-bis(benzyloxy)-2-cyclopropyl-9-methylspiro[benzo[c]chromene-6,1′-cyclobutane] (70.00 mg, 1.00 Eq, 143.3 μmol) was dissolved in MeOH (4 mL). Nickelous chloride hexahydrate (170.2 mg, 5 Eq, 716.3 μmol) was added. SodiumTetrahydroborate (108.4 mg, 101.4 μL, 20 Eq, 2.865 mmol) were added portionwise (gaz evolution). TLC showed no more starting material. The mixture was filtered off and the crude was loaded on silica gel and purified by FC eluent MeOH/DCM 0% to 4% to give 2-cyclopropyl-9-methylspiro[benzo[c]chromene-6,1′-cyclobutane]-3,8-diol (20 mg, 65 μmol, 45%) as a white solid. R_{f} 0.4 MeOH/DCM 4%. MS: m/z: [M+H]+ 309. ¹H NMR (400 MHz, DMSO) δ 9.38 (s, 1H), 9.32 (s, 1H), 7.42 (s, 1H), 7.04 (s, 1H), 6.85 (s, 1H), 6.38 (s, 1H), 2.38 (q, J=9.9 Hz, 2H), 2.28-2.16 (m, 2H), 2.16 (d, J=0.7 Hz, 3H), 2.07-1.90 (m, 2H), 1.82-1.65 (m, 1H), 0.92-0.75 (m, 2H), 0.67 (dt, J=5.5, 2.9 Hz, 2H).

### 23. Synthesis of 9-cyclopropyl-2-methylspiro[benzo[c]chromene-6,1′-cyclobutane]-3,8-diol

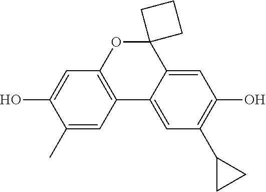 [SMILES: Cc1cc2c(cc1O)OC1(CCC1)c1cc(O)c(C3CC3)cc1-2]

19 was prepared from 2-(4-(benzyloxy)-2-fluoro-5-methylphenyl)-4,4,5,5-tetramethyl-1,3,2-dioxaborolane (see synthesis of 7) and 1-(benzyloxy)-5-bromo-2-cyclopropyl-4-iodobenzene.

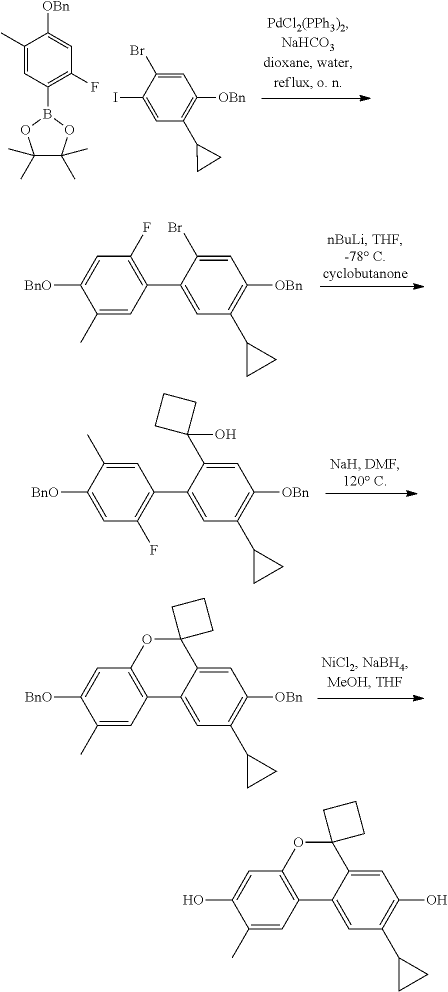

### Synthesis of 1-(benzyloxy)-5-bromo-2-cyclopropyl-4-iodobenzene

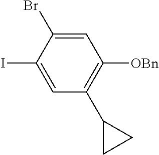 [SMILES: Brc1cc(OCc2ccccc2)c(C2CC2)cc1I]

1-(benzyloxy)-5-bromo-2-cyclopropyl-4-iodobenzene was prepared from commercially available 5-bromo-2-iodophenol in 3 steps.

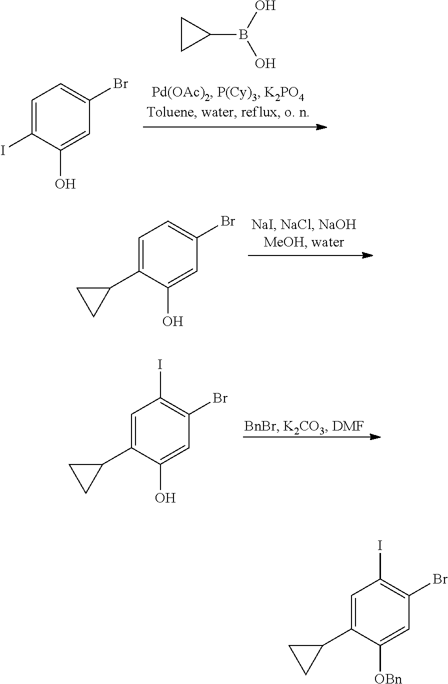

### Step 1: Synthesis of 5-bromo-2-cyclopropylphenol

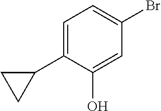 [SMILES: Oc1cc(Br)ccc1C1CC1]

Phosphine, tricyclohexyl-(150.1 mg, 167 μL, 0.16 Eq, 535.3 μmol) and Palladium diacetate (60.09 mg, 0.08 Eq, 267.6 μmol) were suspended and stirred in degassed toluene (308.3 mg, 33.45 mL, 0.1 molar, 1 Eq, 3.345 mmol) for 2 min. 5-bromo-2-iodophenol (1000 mg, 1 Eq, 3.345 mmol) and cyclopropylboronic acid (1.150 g, 4 Eq, 13.38 mmol) were add successively followed by Potassium phosphate, tribasic (2.130 g, 830.9 μL, 3 Eq, 10.04 mmol) dissolved in water (60.29 mg, 16.73 mL, 0.2 molar, 1 Eq, 3.345 mmol). The mixture was heated at 110° C. overnight. NH4Cl saturated solution was added and the mixture was extracted with EtOAc twice. The organic phases were dried over sodium sulfate and concentrated under vacuum. The crude was purified by FC eluent EtOAc/cyclohexane 0% to 35% to give 5-bromo-2-cyclopropylphenol (350 mg, 1.64 mmol, 49.1%) as brownish oil. R_{f} 0.3 eluent EA/cyclohexane 10%. ¹H NMR (400 MHz, DMSO) δ 9.80 (s, 1H), 6.92 (d, J=2.1 Hz, 1H), 6.84 (dd, J=8.2, 2.0 Hz, 1H), 6.69 (d, J=8.2 Hz, 1H), 2.07-1.89 (m, 1H), 0.95-0.79 (m, 2H), 0.68-0.52 (m, 2H).

### Step 2: synthesis of 5-bromo-2-cyclopropyl-4-iodophenol

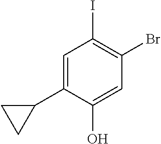 [SMILES: Oc1cc(Br)c(I)cc1C1CC1]

5-bromo-4-iodo-2-methylphenol was prepared from 5-bromo-2-ethylphenol according to procedure described in Beatrice Felber, Francois Dietrich, Helvetica, 2005, vol 88, 120-153. ¹H NMR (400 MHz, CDCl₃) δ 7.49 (d, J=0.9 Hz, 1H), 7.16 (s, 1H), 1.83-1.62 (m, 1H), 1.10-0.86 (m, 2H), 0.62 (ddd, J=5.8, 3.9, 1.9 Hz, 2H).

### Step 3: synthesis of 1-(benzyloxy)-5-bromo-2-cyclopropyl-4-iodobenzene

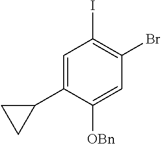 [SMILES: Brc1cc(OCc2ccccc2)c(C2CC2)cc1I]

Benzyl bromide (481.64 mg, 334.9 μL, 1 Eq, 2.8160 mmol) was added to a mixture of 5-bromo-2-cyclopropylphenol (600.00 mg, 1.00 Eq, 2.8160 mmol) and potassium carbonate (778.34 mg, 2 Eq, 5.6320 mmol)benzyl bromide (481.64 mg, 334.9 μL, 1 Eq, 2.8160 mmol) in ACN (14 mL) and the mixture was heated at 50° C. for 3 h. The reaction mixture was filtered off concentrated under vacuum give 1-(benzyloxy)-5-bromo-4-iodo-2-methylbenzene (4 g, 9.9 mmol, 89%) used to the next step without further purification. ¹H NMR (400 MHz, CDCl₃) δ 7.47-7.31 (m, 5H), 7.25 (d, J=0.6 Hz, 1H), 7.14 (s, 1H), 5.06 (s, 2H), 2.16-2.04 (m, 1H), 0.98-0.83 (m, 2H), 0.68-0.56 (m, 2H).

### Synthesis of 19

### Step 1: Synthesis of 4,4′-bis(benzyloxy)-2-bromo-5-cyclopropyl-2′-fluoro-5′-methyl-1,1′-biphenyl

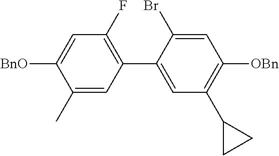 [SMILES: Cc1cc(-c2cc(C3CC3)c(OCc3ccccc3)cc2Br)c(F)cc1OCc1ccccc1]

1-(benzyloxy)-5-bromo-2-cyclopropyl-4-iodobenzene (150 mg, 1 Eq, 350 μmol) and 2-(4-(benzyloxy)-2-fluoro-5-methylphenyl)-4,4,5,5-tetramethyl-1,3,2-dioxaborolane (156 mg, 1.3 Eq, 454 μmol) were dissolved in dioxane (10 mL). Bis-(triphenylphosphino)-palladous chloride (24.5 mg, 0.1 Eq, 35.0 μmol) was added and the solution was degassed for 5 min. then sodium bicarbonate (88.1 mg, 1.05 mL, 1 molar, 3 Eq, 1.05 mmol) was dissolved in water (4 mL) and added dropwise and the mixture was heated at 90° C. 2 h. Water was added and the mixture was extracted with EtOAc twice. The combined organic phases were dried over sodium sulfate and concentrated under vacuum. The crude was purified by FC biotage EtOAc/cyclohexane 0% to 5% to give 4,4′-bis(benzyloxy)-2-bromo-5-cyclopropyl-2′-fluoro-5′-methyl-1,1′-biphenyl (130 mg, 251 μmol, 71.9%) as colourless oil. R_{f} 0.3 EtOAc/cycolohexane 2%. ¹H NMR (400 MHz, CDCl₃) δ 7.55-7.32 (10 m, H), 7.18 (s, 1H), 7.01 (dd, J=8.6, 0.9 Hz, 1H), 6.77 (s, 1H), 6.69 (d, J=11.4 Hz, 1H), 5.12 (s, 2H), 5.09 (s, 2H), 2.31 (s, 3H), 1.10-0.88 (m, 2H), 0.74-0.57 (m, 2H).

### Step 2: Synthesis of 1-(4,4′-bis(benzyloxy)-5-cyclopropyl-2′-fluoro-5′-methyl-[1,1′-biphenyl]-2-yl)cyclobutan-1-ol

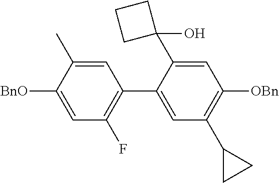 [SMILES: Cc1cc(-c2cc(C3CC3)c(OCc3ccccc3)cc2C2(O)CCC2)c(F)cc1OCc1ccccc1]

nBuLi (74.28 mg, 724.72 μL, 1.6 molar in THF, 3 Eq, 1.1596 mmol) was added to a solution of 4,4′-bis(benzyloxy)-2-bromo-5-cyclopropyl-2′-fluoro-5′-methyl-1,1′-biphenyl (200.00 mg, 1.00 Eq, 386.52 μmol) in THF (7 mL) at −78° C. The mixture was stirred for 30 min at −78° C. then 1-Oxocyclobutane (270.9 mg, 289 μL, 10 Eq, 3.8652 mmol) was added dropwise and the mixture was allowed to warm to rt. over 6 h. NH4Cl saturated solution was added and the mixture was extracted with EtOAc twice. The combined organic phases were dried over sodium sulfate and concentrated under vacuum. The crude was purified by FC eluent EtOAc/cyclohexane 0% to 20% to give 1-(4,4′-bis(benzyloxy)-5-cyclopropyl-2′-fluoro-5′-methyl-[1,1′-biphenyl]-2-yl)cyclobutan-1-ol (60 mg, 0.12 mmol, 31%). R_{f} 0.3 EtOAc/cyclohexane 20%. ¹H NMR (400 MHz, CDCl₃) δ 7.54-7.31 (m, 10H), 7.02 (dd, J=8.9, 0.9 Hz, 1H), 6.88 (s, 1H), 6.67 (s, 1H), 6.61 (s, 1H), 5.15 (s, 2H), 5.08 (s, 2H), 2.34-2.11 (m, 5H), 2.10-1.95 (m, 2H), 1.76-1.68 (m, 1H), 1.63-1.41 (m, 1H), 1.04-0.83 (m, 2H), 0.67 (ddd, J=5.3, 3.0, 2.0 Hz, 2H).

### Step 3: Synthesis of 3,8-bis(benzyloxy)-9-cyclopropyl-2-methylspiro[benzo[c]chromene-6,1′-cyclobutane]

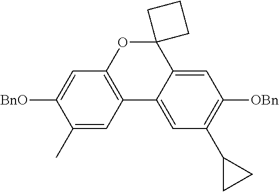 [SMILES: Cc1cc2c(cc1OCc1ccccc1)OC1(CCC1)c1cc(OCc3ccccc3)c(C3CC3)cc1-2]

NaH (14 mg, 60% Wt, 3 Eq, 0.35 mmol) was added to a solution of 1-(4,4′-bis(benzyloxy)-5-cyclopropyl-2′-fluoro-5′-methyl-[1,1′-biphenyl]-2-yl)cyclobutan-1-ol (60 mg, 1 Eq, 0.12 mmol) in DMF (7 mL) at 110° C. stirring continued for 15 min at 110° C. The reaction mixture was cooled down to room temperature. NaHCO₃ 1/2 saturated solution was added and the mixture was extracted with EtOAc once. The organic phase was dried over sodium sulfate. the solvent was evaporated under vacuum and the crude was purified by FC eluent EtOAc/cyclohexane 0% to 10% to give 3,8-bis(benzyloxy)-9-cyclopropyl-2-methylspiro[benzo[c]chromene-6,1′-cyclobutane] (40 mg, 82 mol, 69%) as a white solid. R_{f} 0.7 EtOAc/cyclohexane 5/95. ¹H NMR (400 MHz, CDCl₃) δ 7.61-7.31 (m, 11H), 7.12 (s, 1H), 6.94 (s, 1H), 6.59 (s, 1H), 5.20 (s, 2H), 5.07 (s, 2H), 2.52 (q, J=10.5 Hz, 2H), 2.40-2.29 (m, 3H), 2.28 (s, 3H), 2.05-1.94 (m, 1H), 1.80-1.69 (m, 1H), 1.09-0.94 (m, 2H), 0.79-0.71 (m, 2H).

### Step 4: Synthesis of 9-cyclopropyl-2-methylspiro[benzo[c]chromene-6,1′-cyclobutane]-3,8-diol

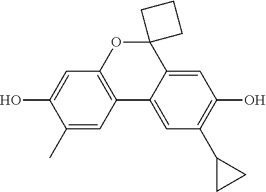 [SMILES: Cc1cc2c(cc1O)OC1(CCC1)c1cc(O)c(C3CC3)cc1-2]

3,8-bis(benzyloxy)-9-cyclopropyl-2-methylspiro[benzo[c]chromene-6,1′-cyclobutane] (40.00 mg, 1.00 Eq, 81.86 mol) was dissolved in MeOH (4 mL). Nickelous chloride hexahydrate (97.28 mg, 5 Eq, 409.3 mol) was added. Sodium Tetrahydroborate (61.94 mg, 57.94 L, 20 Eq, 1.637 mmol) were added portionwise (gaz evolution). TLC showed no more starting material. The mixture was filtered off and the crude was loaded on silica gel and purified by FC eluent MeOH/DCM 0% to 20% to give 9-cyclopropyl-2-methylspiro[benzo[c]chromene-6,1′-cyclobutane]-3,8-diol (10 mg, 32 mol, 40%) as a yellow solid. R_{f} 0.4 MeOH/DCM 4%. MS: m/z: [M+H]+ 309. ¹H NMR (400 MHz, DMSO) δ 9.38 (s, 1H), 9.32 (s, 1H), 7.42 (s, 1H), 7.04 (s, 1H), 6.85 (s, 1H), 6.38 (s, 1H), 2.44-2.35 (m, 2H), 2.26-2.17 (m, 2H), 2.16 (d, J=0.7 Hz, 3H), 2.05-1.86 (m, 2H), 1.79-1.66 (m, 1H), 0.91-0.75 (m, 2H), 0.67 (dt, J=5.5, 2.9 Hz, 2H).

### Example 2: Anti-ferroptotic assay

Anti-ferroptotic activity of compounds was determined by measuring cellular viability after co-treatment with the ferroptosis inducer 1S,3R-RSL 3 (CAS No.:1219810-16-8; hereafter RSL3). Cellular viability was measured using the CellTiter-Glo® 2.0 Assay. The assay provides a homogeneous method to determine the number of viable cells in culture by quantitating the amount of ATP present, which indicates the presence of metabolically active cells.

On day 1, C2C12 myoblasts (ATCC #CRL-1772) where seeded in white-walled, transparent bottom 96 w plates at 1,500 cells/well in regular DMEM medium, added with 10% heat inactivated fetal bovine serum (FBS) and Penicillin-Streptomycin (100 U/mL). On day 2, cells were treated with DMSO at 0.1% (vehicle, n=8 per plate), RSL3 at 1.25 μM as a positive control (n=8 per plate) and test compounds in a 5-points concentration-response curve starting from 50 μM with 2-fold dilutions (n=4 per plate and per concentrations), in presence or absence of RSL3 1.25 μM. On day 3, CellTiter-Glo reagent (2× stock) (Promega) was added, mixed two minutes on orbital shaker and incubated 10 minutes at RT in the dark, before measuring luminescence using a FLUOstar OPTIMA reader (0.25s). The percentage of efficacy (PE) of each test compound concentration corresponds to the rescue in cellular viability compared to RSL3 1.25 μM alone and each test compound concentration alone:

$$P E = ( \frac{\begin{matrix}( signal ( compound [ n μ M ] + RSL 3 [ 1.25 μ M ] ) - \\ average signal ( RSL 3 [ 1.25 μ M ] )\end{matrix}}{average signal ( compound [ n μ M ] )} ) * 100$$

A score of 0% means that the compound does not have any anti-ferroptotic activity. A score of 100% means that the compound rescues completely cellular viability and has the maximal anti-ferroptotic activity possible.

The concentration at which compounds show 50% efficacy against ferroptosis-induced cell death (EC50) was calculated with GraphPad Prism v9.4.0. Using non-linear regression of log 10 transformed concentrations (log(agonist) vs. response—Variable slope (four parameters)) with the constraint of Bottom=0 and Top=100. pEC₅₀ values correspond to −log 10 of the EC₅₀ values.

### INCORPORATION BY REFERENCE

All of the U.S. patents and U.S. and PCT patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A compound of Formula (I): 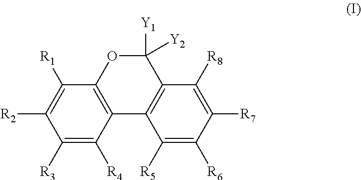
wherein
Y₁ and Y₂ are each alkyl; or taken together with the carbon to which they are bonded combine to form an unsubstituted or substituted spiro cycloalkyl;
R₁, R₄, R₅, and R₈ are independently selected from —H and halogen;
R₂ and R₇ are independently selected from —H, —OH, —OAc, —NH₂, halogen, —CN, —CF₃, —CO₂H, —NO₂, —NHAc, alkyl, haloalkyl, alkenyl, alkynyl, cycloalkyl, heterocycloalkyl, alkylamino, alkyl-R₉, alkenyl-R₉, alkynyl-R₉, —OR₁₀, —NHR₁₀, —NR₁₁C(O)R₁₂, —C(O)NR₁₁R₁₂, and —NR₁₁SO₂R₁₂;
R₃ and R₆ are independently selected from alkyl and cycloalkyl;
each occurrence of R₉ is independently selected from OH, NH₂, O-alkyl, O-alkyl-O-alkyl, alkylamino, NHC(O)-alkyl, N(CH₃)C(O)-alkyl, NHSO₂-alkyl, N(CH₃)SO₂-alkyl, aryl, heteroaryl, cycloalkyl, and heterocycloalkyl;
R₁₀ is selected from alkyl, hydroxyalkyl, aminoalkyl, alkyl-O-alkyl, alkyl-O-alkyl-OH, alkyl-O-alkyl-O-alkyl, alkenyl, alkynyl, heteroarylalkyl, heteroarylalkyl, alkyl-cycloalkyl, alkyl-heterocycloalkyl, aryl, heteroaryl, cycloalkyl, heterocycloalkyl, SO₃H, SO₂-alkyl, and SO₂-haloalkyl;
each occurrence of R₁₁ is selected from H and alkyl; and
each occurrence of R₁₂ is selected from alkyl, alkenyl, alkynyl, cycloalkyl, heterocycloalkyl, O-alkyl, aminoalkyl, heteroarylalkyl, heteroarylalkyl, alkyl-cycloalkyl, and alkyl-heterocycloalkyl;
provided that when R₁, R₄, R₅, and R₈ are each —H, R₂ and R₇ are each —OH, and R₃ and R₆ are each CH₃, then Y₁ and Y₂ are not each -Me or are not taken together with the carbon to which they are bonded to form an unsubstituted spiro cyclobutyl;
or a pharmaceutically acceptable salt thereof.

2. The compound of claim 1, wherein R₃ and R₆ are alkyl.

3. The compound of claim 2, wherein Y₁ and Y₂ are each independently C₁-C₄ alkyl.

4. The compound of claim 2, wherein Y₁ and Y₂ are each —CH₃.

5. (canceled)

6. The compound of claim 2, wherein Y₁ and Y₂ taken together with the carbon to which they are bonded combine to form an unsubstituted spiro cyclopropyl, cyclobutyl, or cyclopentyl.

7. The compound of claim 1, wherein R₃ and R₆ are each independently C₁-C₄ alkyl.

8. The compound of claim 7, wherein R₃ and R₆ are each independently selected from —CH₃ and —CH₂CH₃.

9. (canceled)

10. (canceled)

11. (canceled)

12. The compound of claim 1, wherein R₃ and R₆ are each independently C₃-C₅ cycloalkyl; or one of R₃ and R₆ is C₁-C₄ alkyl and the other of R₃ and R₆ is C₃-C₅cycloalkyl.

13. The compound of claim 12, wherein R₃ and R₆ are each cyclopropyl; or one of R₃ and R₆ is —CH₃ and the other of R₃ and R₆ is cyclopropyl.

14. (canceled)

15. (canceled)

16. The compound of claim 1, having the structure selected from:

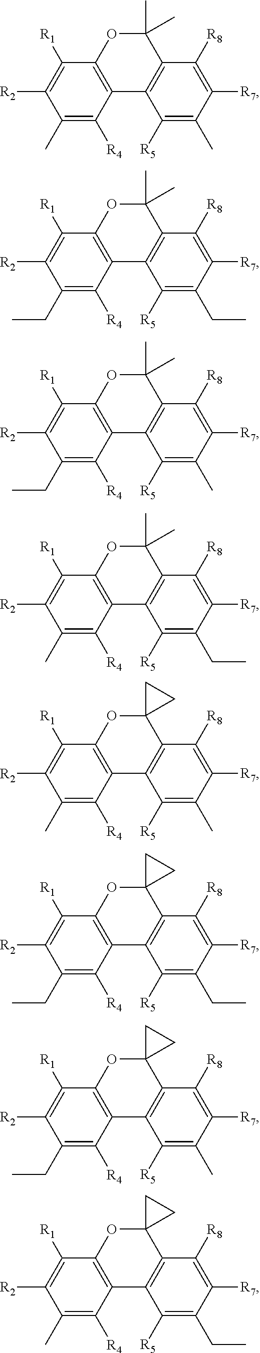

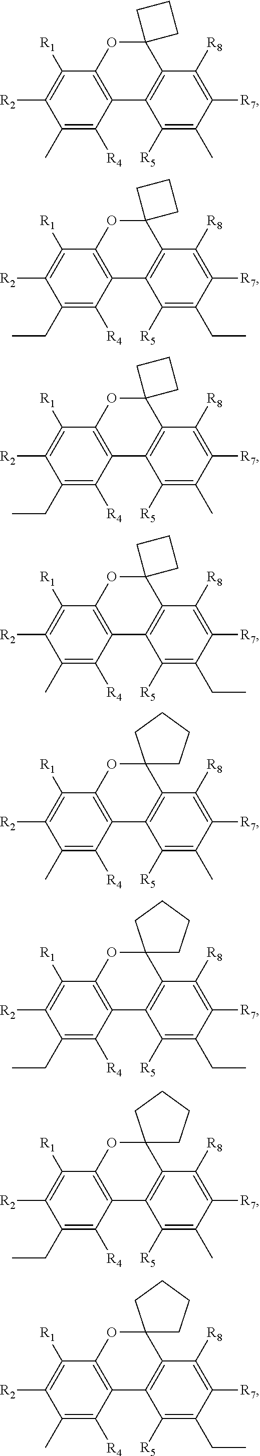

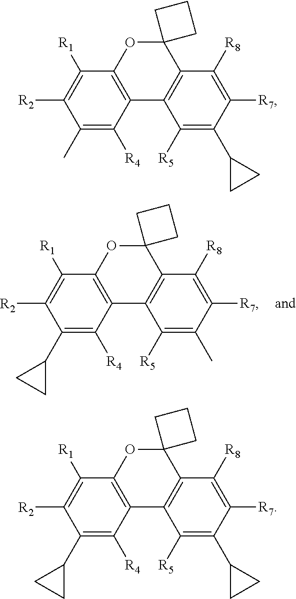

17. (canceled)

18. The compound of claim 1, wherein R₂ and R₇ are independently selected from —OH, —NH₂, alkylamino, and —OR₁₀.

19. The compound of claim 18, wherein R₂ and R₇ are each OH; R₂ is —OH and R₇ is —OCH₃; R₇ is —OH and R₂ is —OCH₃.

20. (canceled)

21. (canceled)

22. The compound of claim 18, wherein R₂ is selected from —NH₂, —NHCH₃, and —NH(CH₃)₂; and R₇ is OH; or R₇ is selected from —NH₂, —NHCH₃, and —NH(CH₃)₂; and R₂ is OH.

23. (canceled)

24. The compound of claim 1, wherein R₁, R₄, R₅, and R₈ are each —H.

25. The compound of claim 1 having the structure of a compound selected from: 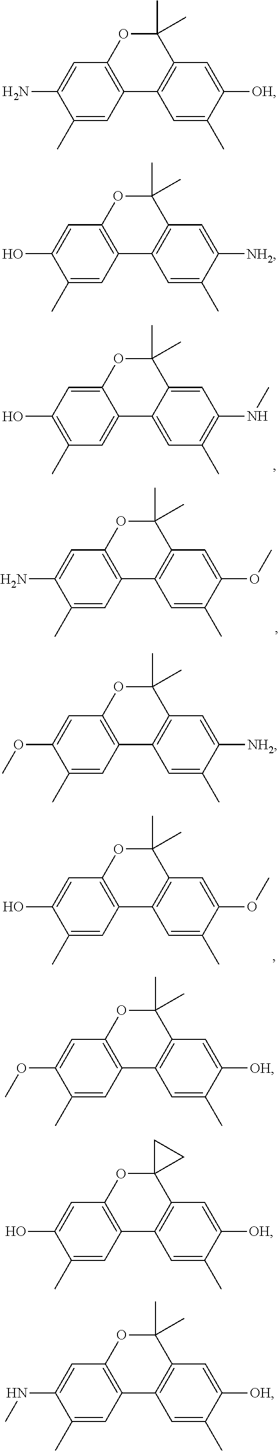 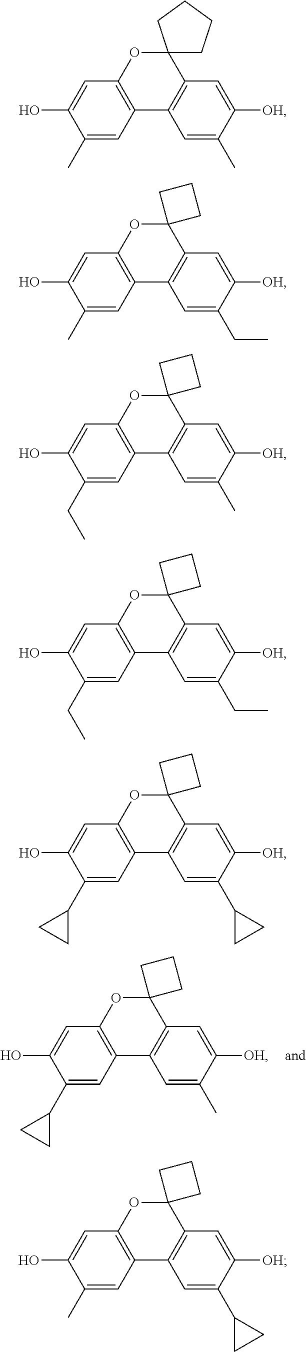
or a pharmaceutically acceptable salt thereof.

26. A compound of Formula (II): 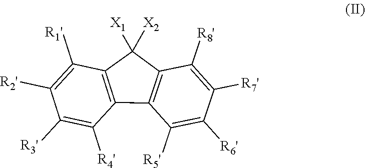
wherein
X₁ and X₂ are each alkyl; or taken together with the carbon to which they are bonded combine to form an unsubstituted or substituted spiro cycloalkyl;
R₁′, R₄′, R₅′, and R₈′ are independently selected from —H, —OH, —NH₂, alkyl, and halogen;
R₂′, R₃′, R₆′, and R₇′ are independently selected from —H, —OH, —OAc, —NH₂, halogen, —CN, —CF₃, —CO₂H, —NO₂, —NHAc, alkyl, haloalkyl, alkenyl, alkynyl, cycloalkyl, heterocycloalkyl, alkylamino, and —OR₈′; and
R₈′ is selected from alkyl, hydroxyalkyl, aminoalkyl, aryl, heteroaryl, cycloalkyl, and heterocycloalkyl;
or a pharmaceutically acceptable salt thereof.

27.-42. (canceled)

43. The compound of claim 26 having the structure of a compound selected from: 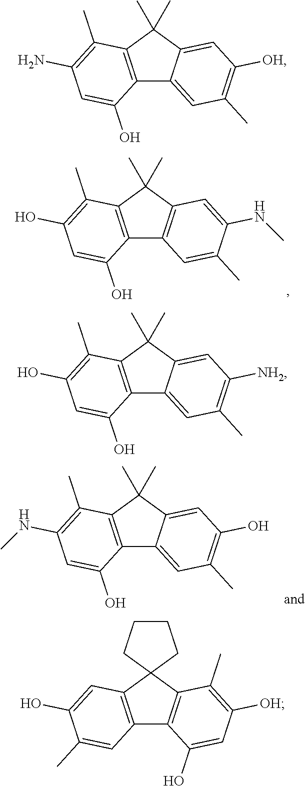
or a pharmaceutically acceptable salt thereof.

44. A pharmaceutical composition comprising a compound of claim 1; and a pharmaceutically acceptable carrier.

45. A method of inhibiting ferroptosis or of treating an inflammatory disease, neuronal disease or neurodegenerative disease at least partially mediated by ferroptosis, comprising administering to a subject in need thereof an effective amount of a compound of claim 1.

46. A method of inhibiting ferroptosis or of treating an inflammatory disease, neuronal disease or neurodegenerative disease at least partially mediated by ferroptosis, comprising administering to a subject in need thereof an effective amount of a compound of claim 26.
